(19) United States
(12) Patent Application Publication
(10) Pub. No.: US 2023/0061483 A1
(43) Pub. Date: Mar. 2, 2023

(54) MICROTOPOGRAPHIES AND USES THEREOF

(71) Applicant: THE UNIVERSITY OF NOTTINGHAM, Nottingham, Nottinghamshire (GB)

(72) Inventors: Morgan Alexander, Nottingham, Nottinghamshire (GB); Paul Williams, Nottingham, Nottinghamshire (GB); Amir Ghaemmaghami, Nottingham, Nottinghamshire (GB)

(73) Assignee: THE UNIVERSITY OF NOTTINGHAM, Nottingham, Nottinghamshire (GB)

(21) Appl. No.: 17/799,910

(22) PCT Filed: Feb. 15, 2021
(86) PCT No.: PCT/IB2021/051275

§ 371 Date: Aug. 15, 2022

(30) Foreign Application Priority Data
Feb. 13, 2020 (GB) 2002010.3

(51) Int. Cl. C12N 5/00

(52) U.S. Cl. CPC C12N 5/0068; C12N 2535/10; C12N 2533/30

(57) ABSTRACT

A microtopography system for modulating one or more cellular processes on a surface is described. The microtopography system comprising: a repeated microtopographic pattern, said microtopographic pattern comprising: an array of repeated micropillars applied to a surface of a product, said micropillars being formed of surface structures between 1-100 μm in height, and 1-50 μm in width. The microtopographic pattern acts to modulate one or more cellular processes on the surface.

The present invention relates to methods of identifying microtopographies which modulate cellular processes, uses of such microtopographies and products comprising them on their surface.

The developments made in the biomaterials field over the past 25 years (Bhat & Kumar, 2013) and the rise of newly available materials have prompted their use as an essential component of modern medicine and industrial activity utilising biological processes. Used for modulating cell proliferation and healing in regenerative medicine, for drug delivery, to fabricate a broad range of medical devices, and for producing molecules and products of interest on an industrial scale, biomaterials play an important role in disease management and healthcare improvement in day to day practice as well as in a variety of industrial areas. Moreover, there is a growing demand to improve the levels of wellbeing in rapidly expanding and ageing populations (Holzapfel et al., 2013), as well as to find new, cheaper ways to exploit biological processes for industrial purposes. Such demands urge the rise of a new era of biomaterials. Despite the progress made to date, a poor understanding of the complex cellular mechanisms and environment at a surface-cell interface has hampered the rational design of biomaterials. It remains important to understand the relationship between the surface topography and surface chemistry of materials and its influence on biological processes in order to exploit these for the benefit of mankind.

For example, when given a suitable environment for adhesion, cells such as bacteria or immune cells can attach to surfaces and increase or decrease their metabolic and/or proliferative activities. In the context of materials implanted into a subject, pathogenic bacteria may attach to a surface of the implanted material and form a biofilm which leads to clinical infection (Davies, 2003). In other industries, food spoilage and contamination of the surface and local area may occur upon bacterial attachment. In the context of immunomodulation, the local environment of an implanted material may be able to influence immune rejection the implanted material via influencing the polarisation of immune cells surrounding or attached to the surface of the implanted material.

Most attempts to manage the issue of the unwanted proliferation of microbes have focused on the incorporation of antimicrobials to materials with the aim of killing or biologically inhibiting the growth of microbes (Zhao et al., 2009; Goodman et al., 2013). However, the main drawbacks of these strategies are their limited long-term efficacy and the enhancement of antimicrobial resistance (Swearingen et al., 2016). Similarly, pharmaceutically induced polarisation of immune cells relies on expensive-to-research and produce small molecules or biologics, which have many drawbacks such as toxic off-target effects, low efficacy, difficulty in applying to target sites, regulatory hurdles, and in the field of oncology the promotion of tumour resistance.

There is thus a need in the industry to develop alternative or improved methods to modulate cellular processes such as cell attachment or immune cell polarisation, which have a long-term efficacy, which are relatively cheap to make, which have a low toxicity profile do not force selective pressures on organisms.

According to an aspect, the invention provides a microtopography system for modulating one or more cellular processes on a surface, said microtopography system comprising a repeated microtopographic pattern, said microtopographic pattern comprising an array of repeated micropillars applied to a surface of a product, said micropillars being formed of surface structures between 1-100 μm in height, and 1-50 μm in width, wherein said microtopographic pattern acts to modulate one or more cellular processes on the surface.

In an embodiment, the micropillar may be about 1-100 μm in height (vertical), such as about between 5-45 μm, 10-40 μm, 15-35 μm, 20-30 μm, 25 μm, or 50-100 μm in height. In one preferred embodiment the micro-pillar may be approximately 10 μm in height.

Similarly, the micropillars may be between 1-100 μm in width (lateral), such as 2-45 μm, 3-40 μm, 4-35 μm, 5-30 μm, 10-25 μm, 15-20 μm, or 50-100 μm. In one embodiment the micropillars are approximately 3 μm in width, such as 3.0+/−0.6 μm. Suitably, a micro-pillar may be 3-23 μm wide laterally and about 10 μm in height, such as 9.1+/−0.6 μm

In one embodiment the microtopography of the micropillars above the underlying surface may have a mean area below 50 μm². In other embodiments, the micropillars have an eccentricity of <1, and preferably less than 0.5, preferably between 0.01-0.49, more preferable between 0.1-0.4, most preferably between 0.2-0.3.

Typically the micropillars are shaped according to a topography determined using a screening technique of possible primitive shape combinations. Said primitive combinations may comprise one or more of rectangles (including square), circles, triangles or other primitive shapes. Said shapes may be combined using a computational algorithm to generate a hybrid shape or micropillar that does not resemble the original primitives. It can be appreciated that such a hybrid shape may be a single conjoined shape, or may be a collection of shapes, in which case the micropillar is considered to include all shapes in the collection. The micropillars are then arranged on the surface in a repeating patterned array. Accordingly, in addition to interaction between the shapes or morphology of a single micropillar, cellular processes may be influenced by adjacent micropillars.

In another aspect, there is provided a method of screening for a microtopography which modulates one or more cellular processes, wherein the method comprises:
i. Applying at least one microtopography to a surface;
ii. Culturing one or more first set of cells on the surface with said microtopography applied to it, and culturing a matching number and type of cells of a second set of cells on a reference surface;
iii. Measuring or detecting the level of one or more cellular processes of the first and second set of cells;
iv. Comparing the level of the one or more measured or detected cellular processes of the first and second set of cells; and
v. Determining whether the level of each of the one or more measured or detected cellular process between the first and second set of cells is modulated either positively or negatively.

Advantageously, the invention allows the application of microtopographies to surfaces such as existing biomaterials, clinical materials and tools, as well as industrial materials to modulate cellular activities such as microbial attachment or immune activity on the surfaces applied thereto. Surfaces with microtopographies applied possess a low toxicity profile, and this approach reduces costs and need for expensive new material discovery, and provides the opportunity to combine approaches with other surface modifications such as chemical coating, and/or antimicrobial agent treatment to achieve a desired effect on a level of a cellular process.

In a third aspect, the invention provides a method of modulating one or more cellular processes at a surface, wherein the method comprises applying a microtopography to said surface.

In a fourth aspect, the invention provides a product with a surface on which a microtopography has been applied, for use in modulating one or more cellular processes.

In an embodiment, the one or more cellular processes of the first three aspects of the invention comprises or consists of cell attachment. The cells may be eukaryotic or prokaryotic cells. The prokaryotic cells may be bacterial cells. The eukaryotic cells may be innate immune cells such CD14+ monocytes or APCs, or adaptive immune cells such as T-cells or non-immune cells such as fibroblasts. The APCs may be human or non-human mammal APCs. The APCs may be macrophages or Dendritic Cells.

In another embodiment, the one or more cellular processes of the first three aspects of the invention comprises or consists of immune activity of cells. The cells may be innate immune cells such APCs or adaptive immune cells such as T-cells or non-immune cells such as fibroblasts. The APCs may be human or non-human mammal APCs. The APCs may be macrophages or Dendritic Cells.

In another embodiment, the one or more cellular processes comprises or consists of both cell attachment and immune activity.

In a fifth aspect, the invention provides a product with a surface on which a microtopography has been applied, wherein said microtopography modulates cell attachment to the surface of said product and/or immune activity of the attached cells.

The immune activity of cells in the microenvironment of the surface on which a microtopography has been applied may also be modulated. The cell attachment may be increased or decreased compared to the surface of a reference surface. The immune activity may be increased or decreased compared to the surface of a reference surface. The cells may be innate immune cells such as APCs or adaptive immune cells such as T-cells or non-immune cells such as fibroblasts. The APCs may be human or non-human mammal APCs. The APCs may be macrophages or Dendritic Cells. The APCs may be a mixture of macrophages and Dendritic Cells

In an embodiment, a product is for use in preventing or reducing the risk of biofilm formation. The biofilm may be caused by one or more of Pseudomonas spp., Staphylococcus spp., Bacillus spp., Lactobacillus sp., Proteus spp., Enterobacter spp., Escherichia coli, Klebsiella spp., Salmonella spp., Listeria spp., Yersinia spp., Legionella spp, Clostridium spp., Acinetobacter spp. For example, a bacterial infection may be caused by one or more of Pseudomonas aeruginosa, Staphylococcus aureus, Proteus mirabilis, Acinetobacter baumannii.

In an embodiment, the product is for use in preventing or treating an infection. The infection may be caused by one or more of a bacteria, a virus, a fungi, a protozoan. The infection may be caused by one or more of Pseudomonas spp., Staphylococcus spp., Bacillus spp., Lactobacillus sp., Proteus spp., Enterobacter spp., Escherichia Coli, Klebsiella spp., Salmonella spp., Listeria spp., Yersinia spp., Legionella spp, Clostridium spp., Acinetobacter spp. For example, a bacterial infection may be caused by one or more of Pseudomonas aeruginosa, Staphylococcus aureus, Proteus mirabilis, Acinetobacter baumannii. Suitably, the bacterial infection may be the result of biofilm formation.

In an embodiment, the product is for use in preventing rust formation by increasing bacterial cell attachment. The product may be for use in wound dressings by increasing bacterial cell attachment to the dressing and removing bacterial cells from the wound. The product may be for use in coating a cell culture dish, to promote adherence, viability and/or growth of adherent cells such as skin cells and fibroblasts by increasing cell attachment. The product may be a food container, food packaging, or any other food preparation or storage surface, for use in preventing food spoilage or contamination by reducing/resisting bacterial cell attachment. The product may be for use in encasing any other product, to prevent contamination of said encased product by reducing/resisting bacterial cell attachment. The product may be for use in improving the output or efficiency of an industrial process, such as chemical of biochemical production or enzymatic metabolization of a substrate, by increasing the attachment of cells to the surface, wherein the cells undertake or contribute to the industrial process. The product may be a surface of food processing equipment such as vats and pipework. The product may be a surface of water systems such as those used in food manufacture, healthcare water loop systems, water containers (i.e. domestic/industrial plumbing, waste water management). The product may be a surface of products in the Beverage industry such as beer lines. The product may be a surface such as touch-screen displays, windows such as those at aquariums.

In a seventh aspect, the invention provides a product with a surface on which a microtopography has been applied, for use in treating or preventing an immune disease/disorder or an infection in said subject, by modulating the attachment and/or immune activity of APCs in a subject. The immune activity of APCs may be increased or decreased compared to the surface of a reference surface.

In an embodiment, the APC is a macrophage. In another embodiment, the APC is a Dendritic Cell. In another embodiment, the APC is a Dendritic Cell.

In an embodiment, the immune disease/disorder is selected from the following: transplant rejection, Graft Versus Host Disease (GVHD), psoriasis, eczema, rheumatoid arthritis, a cancer, immunosuppression, systemic lupus erythematosus, inflammatory bowel disease, Crohn's disease, multiple sclerosis, Type I diabetes, Guillain-Barre syndrome, fibrosis, chronic non-healing wounds or medical device rejection.

In an embodiment, the infection is caused by one or more of a bacteria, a virus, a fungi, a protozoan. The infection may be caused by one or more of Pseudomonas spp., Staphylococcus spp., Bacillus spp., Lactobacillus sp., Proteus spp., Enterobacter spp., Escherichia coli, Klebsiella spp., Salmonella spp., Listeria spp., Yersinia spp., Legionella spp, Clostridium spp., Acinetobacter spp. For example, a bacterial infection may be caused by one or more of Pseudomonas aeruginosa, Staphylococcus aureus, Proteus mirabilis, Acinetobacter baumannii. Suitably, the bacterial infection may be the result of biofilm formation.

In an embodiment of any aspect, the immune activity is cytokine production. In an embodiment, the immune activity is phagocytosis. In an embodiment, the immune activity is cross-presentation. In an embodiment, the immune activity is CD14+ monocyte differentiation into a macrophage. In an embodiment, the immune activity is macrophage activation. In an embodiment, the immune activity is macrophage polarisation to an M1 or M2 macrophage. In an embodiment, the immune activity is DC maturation and/or activation.

In an embodiment of any aspect, the microtopography of any of the second to the sixth aspects of the invention is identified using the method of the second aspect of the invention. The microtopography may be identified as modulating a cellular process of interest either positively or negatively.

In an embodiment of any aspect, said surface may be placed in a location where the desired cellular process modulation is required. This may be a location where the surface is likely to come into contact with a cell of interest. The cell of interest may be the same as the first and second set of cells of the first aspect of the invention.

Suitably, a microtopography applied to a product or product for use according to any of the above aspects may have been identified as suitable for the use according to any of the methods of the invention.

Microtopographies

A microtopography may be assembled in periodical repetitions of a specific micro-pillar in a defined space, for example in a micro-well. Such a micro-well (also referred to herein as a TopoUnit) may have pre-defined dimensions, and may be present on a chip which comprises multiple micro-wells. Suitable dimensions may be about 500×500 μm, about 300 μm by 300 μm, or about 290 μm×290 μm. Each micro-well may be surrounded by a wall, for example which is about 40 μm tall. Each chip may comprise about 66 by about 66 wells of the same dimensions.

A microtopography may be constructed from a polymer, including a clinically relevant polymer such as polystyrene, polyurethane or Cyclic olefin copolymer (COC). A microtopography may be applied to a well using a mould which has the inverse structure of said microtopography. The microtopography may be applied to the surface of a well by hot embossing.

The microtopography applied to the surface of a well may be subjected to oxygen plasma etching to reduce the hydrophobicity of the material.

The features, including surface chemistry, of a microtopography applied to the surface may be confirmed using a variety of techniques known to the skilled person, for example spectrometric and/or spectroscopic techniques, such as time-of-flight secondary ion mass spectrometry (ToF-SIMS), in situ mass spectrometer and X-ray photoelectron spectroscopy (XPS). A combination of techniques may be used to confirm the surface chemistry of the microtopography.

A microtopography may be applied to a pre-existing surface, or a surface may be constructed to comprise a given microtopography as a principle of its construction.

As used herein, the term ‘reference surface’ refers to a surface in which no specific microtopography has been applied. Such a reference surface may be flat and/or smooth.

The microtopographies screened may be classified into groups, for example by collating the features of a defined number of microtopographies which give a desired outcome on the modulation of a cellular process of interest, for example the top 50, top 100, or top 200 microtopographies which increase the level of cellular process of interest, and the top 50, top 100, or top 200 microtopographies which decrease the level of cellular process of interest. Computational tools may be applied to identify key surface parameters, for example size and organisation of the primitive features in a micro-pillar. The information can then be used to create a predictive model to suggest microtopographies which provide the desired modulation of the cellular process of interest.

Measurement or Detection of Cellular Processes

The skilled person will understand that a cellular process measured, detected or modulated can relate be any cellular activity which can be measured or observed, for example directly or indirectly, visually, or numerically. Such cellular processes may include one or more of cell attachment, cell differentiation, cell motility, cell viability, cell metabolism, cell pluripotency, enzymatic activity, production of specific compounds or metabolites, protein expression, cellular proliferation, DNA replication, cell signalling, cell morphology, immune activity (interchangeably used with the word ‘immunomodulation’).

The skilled person will also understand that a number of methods, readily available at their disposal using common general knowledge, may be utilised in order to measure or detect a cellular process of interest. Such methods may include fluorescence microscopy such as confocal microscopy, other fluorescence based techniques such as FACS and spectroscopy, qRT-PCR, single cell RNA seq, mass spectrometry or other protein quantification methods, western blotting, ELISA, assays to determine the metabolic activity of a cell such as glucose metabolism and respiratory burst, biological assays such as cell survival assays, cell adhesion and protein/particle uptake assays.

The modulation of a cellular process may refer to the increase or decrease of the level of that cellular process measured or detected when compared to the level of that cellular process measured or detected of a control condition, such as a reference surface as described above. The modulation may be determined to be increased or decreased only when a threshold value relative to the control condition is reached. One or a number of parameters may be considered when establishing a relevant threshold value. Thus, a threshold value may be in the units corresponding to the method used to measure or detect the cellular process. Where multiple parameters are measured and considered to establish the threshold value, arbitrary units may be given. The threshold value may be subject to statistical analysis. The threshold value may be dependent upon the exact cellular process measured or detected. The skilled person will readily understand that the nature of the cellular process measured or detected will influence both the method of measurement or detection and any threshold required to make a determination as to whether the cellular process is modulated relative to the control condition.

Cells

The one or more cells cultured in the method of screening according to the invention, or in which the one or more cellular processes are modulated, may be prokaryotic or eukaryotic cells.

Prokaryotic cells may be bacterial cells, such as a pathogenic bacterial cells or bacterial cells used in industrial processes. Prokaryotic cells may be Gram-positive or Gram-negative. Bacterial cells may be one or more of Pseudomonas spp., Staphylococcus spp., Bacillus spp., Lactobacillus sp., Proteus spp., Enterobacter spp., Escherichia coli, Klebsiella spp., Salmonella spp., Listeria spp., Yersinia spp., Legionella spp, Clostridium spp., Acinetobacter spp. For example, bacterial cells may be one or more of Pseudomonas aeruginosa, Staphylococcus aureus, Proteus mirabilis, Acinetobacter baumannii.

Eukaryotic cells may be mammalian or non-mammalian cells. Non-human cells may be fungi cells. Mammalian cells may be human cells, such as cancer cells, immune cells, skin cells, fibroblasts. Immune cells may be monocytes, Antigen Presenting Cells (APCs) such as macrophages or dendritic cells, or immune cells may be CD4+ T-cells, CD8+ T-cells, B-Lymphocytes, Natural Killer (NK) cells.

Cells cultured in the method of screening according to the invention will be cultured in their preferred culture medium and conditions. The skilled person will readily be able to derive the required conditions from the common general knowledge.

Cell Attachment

The extracellular sensing and attachment of cells to surfaces may induce intracellular signalling, leading to metabolic, protein expression and phenotypic changes which can direct biological activities and processes such as immune activity and cell proliferation.

In the context of bacterial attachment to surfaces, a community of microorganisms living may establish on a surface from a planktonic state to form a biofilm, leading to clinical infection. Once the microorganisms attach to the surface, a complex process initiates in which micro-colonies are formed, and cell-cell communication and quorum sensing occurs. Such biofilms are an issue in a variety of situations, particularly in the medical field, where implanted devices or prosthetics which are difficult to remove or exchange see the accumulation of pathogenic microorganisms on a biofilm and the progression of pathogenesis. Biofilms may also form on surfaces which come into contact with food, causing general hygiene issues. Typically, bacteria attach to surfaces using specialised structures such as flagella and pili which are formed of proteins such as adhesins, as well as by hydrodynamic and electrostatic interactions. Polysaccharides, lipopolysaccharide and glycoproteins may also contribute to the attachment.

The attachment of APCs such as DCs and macrophages to a surface may be mediated by specialised proteins, such as integrins, a family of cell surface receptors. Exemplary integrins are V beta 3 (αvβ3) and alpha V beta 5 (αvβ5). Intracellular cytoskeletal movement may also contribute to the ability of an APC to adhere to a surface.

The level of cell attachment may be measured or detected using specific markers. For example, a bacteria or APC may recombinantly express a fluorescent marker, which can be viewed under a microscope. Similarly, fluorescence microscopy to endogenously expressed markers may be used to determine the level of cell attachment.

Immune Modulation

Modulation of immune activity is currently at the forefront of modern medicine, and is seen as the future of the industry to tackle treatment of infections, inflammatory diseases and cancer. Current strategies to control the activity of immune cells include the use of pharmacological small molecules, biologics and even genetic engineering of patient's cells to train them to recognise antigenic targets of interest. All these strategies are extremely expensive, can have toxic side effects and many force directed evolution of pathogens and cancers.

Depending on the circumstance or indication, either an upregulation of immune activity or a downregulation of immune activity can be desired. For example, in an inflammatory disease, the downregulation of inflammatory responses is desired, whilst in infectious scenarios, the upregulation of immune activity of certain cells, such of APCs is highly desirable.

Macrophages, which differentiate from monocytes, represent a heterogeneous population that are present in nearly all tissues of the body and as such, encounter a variety of environments and stimuli, both chemical and physical, and initiates specific inflammatory or healing responses to such stimuli. Similarly, Dendritic Cells, so called ‘sentinels of the immune system’ are specialised APCs which are able to uniquely undertake the process of cross presentation, whereby they ingest and process antigens to present to T-cells, thereby initiating an appropriate adaptive immune response. Upregulating such activities of APCs is clearly desirable in situations such as potential infection, whereas the activity of these cells largely contributes to inflammatory diseases and transplant rejection, so would preferably be downregulated in such circumstances. It is therefore extremely desirable to be able to modulate the activity of these cells in a given environment.

However, little is known about the effect of tissue microenvironment topography and spatial arrangements on APC function and behaviour. The ability to modify or modulate the activation status of APCs, such as the polarisation status of macrophages and activation and maturation of dendritic cells, is emerging as an important new approach to tackle inflammatory diseases or to induce a targeted immune response to pathogens.

The skilled person will also appreciate that the immune activity of cells in the microenvironment of the surface on which a microtopography has been applied may be modulated, either directly as a result of sensing and signalling induced by attachment to the microtopography, or indirectly through cell-cell signalling initiated from cells which are either attached to the microtopography or which are in close proximity to the microtopography.

Immune activity may be measured by the expression of specific markers in a set of subset of cells, or the observable morphology of specific cells. The skilled person will understand that many biological methods, tools and markers are at their disposal to directly or indirectly measure the immune activity of cells, including soluble molecule production and secretion such as cytokine production, cell surface and intracellular protein expression, changes in morphology, cell adherence, mRNA levels and the oxidative state of the cells.

The skilled person will understand that some markers are inflammatory markers (increased immune activity), whilst some are anti-inflammatory or wound healing markers (decreased immune activity), and that the increase in an inflammatory marker would contribute to an increased up upregulated immune activity, whilst an increase in an anti-inflammatory marker would contribute to a decreased immune activity, and vice versa. Additionally, attachment of DCs may lead to their maturation, and the activation of such cells may require the presence of an antigen.

CD14+ monocytes may differentiate into macrophages. Macrophages may be classified as M0 (resting), or polarised to M1 (pro-inflammatory), or M2 (anti-inflammatory).

Classically activated macrophages (pro-inflammatory) are classified as ‘M1’. A suitable marker for M1 macrophages is the expression and/or secretion of TNFα calprotectin. Other markers may include CD86, MHCII, CD25. Alternatively activated macrophages (anti-inflammatory) are classified as ‘M2’. A suitable marker for M2 macrophages is the expression and/or secretion of IL-10, or expression of the mannose receptor. Resting macrophages are classified at M0, and may be classified as such when compared to a polarised M1 or M2 macrophage. A mixture of markers may be used to determine the activation state of a macrophage.

Mature DCs may be identified via upregulated cell surface expression of markers such as CD80, CD86 and MHC-II compared to naïve, non-mature dendritic cells. Similarly, activated DCs may be identified via upregulation of markers such as CD40. A mixture of markers may be used to determine the activation and/or maturation state of DCs.

The skilled person will understand that the marker used to identify the maturation/activation state of an APC will depend on the nature (subset) and location of the APC. A mixture of markers may be used to determine the immune activity of cells.

Medical Uses

The skilled person will recognise that an immune disorder/disease is any disease or disorder in a subject characterised by aberrant immune cell activity, including both over-active and suppressed immune activity, compared to a healthy individual. Exemplary immune disorders/diseases may be an inflammatory disease, immunosuppression, transplant rejection, medical device rejection. Suitably, the immune disorder/disease may be rheumatoid arthritis, systemic lupus erythematosus, inflammatory bowel disease, Crohn's disease, multiple sclerosis, Type I diabetes, Guillain-Barre, psoriasis, cancer, eczema, asthma.

In the case of preventing or reducing transplant rejection and/or GVHD, the surface of a product, such as a prosthetic, implantable medical device, cell culture dish coating, or biodegradable and/or porous protective sheet may be constructed with, or have applied to it, a microtopography which has been identified or predicted to downregulate monocyte and/or APC attachment and/or pro-inflammatory immune activity. Such a product may be placed partially or substantially (for example covering about 10%, about 20%, about 30%, about 40%, about 50%, about 60%, about 70%, about 80%, about 90%, about 95%, about 98%, about 99%) around the organ or tissue transplanted. This would reduce the inflammatory response of APCs which recognise the transplant or graft as foreign, and thus reduce the likelihood of rejection of the transplant or foreign material. The microtopography may have been identified or predicted to have the desired properties using a method of screening of the invention.

In the case of preventing or reducing the risk of biofilm formation, the surface of a product, for example a prosthetic or implantable medical device, may be constructed with, or have applied to it, a microtopography which has been identified or predicted to downregulate or resist cell attachment. The microtopography may have been identified or predicted to have the desired properties using a method of screening of the invention.

In an embodiment of any aspect of the invention, the product may be one or more of the following: an implantable medical device, prosthetic, surgical tool, dental tool or dental device. For example, the product may be a catheter, dental screw, knee joint replacement, hip joint replacement, heart valve replacement, a stent, pacemaker, glucose sensor, contraceptive implant, breast implant, Implantable Cardioverter Defibrillators, spinal screws/rods/artificial discs, contact lenses, different types of shunts and stents prone to fibrosis and infection (e.g. nasolacrimal stents), wound care products

A product described herein ‘for use’ in any method or purpose, may also refer to ‘use of’ that product for said method or purpose.

The skilled person will appreciate that preferred features of any one embodiment and/or aspect of the invention may be applied to all other embodiments and/or aspects of the invention.

### BRIEF DESCRIPTION OF THE FIGURES

FIG. 1—Bacterial attachment assay topographies for P. aeruginosa and S. aureus

(a) Bacterial attachment assay procedure. (b) Ranking of topographies according to the mean fluorescence intensity per TU after P. aeruginosa and S. aureus attachment to PS TopoChips (n=22 and n=6 respectively). The dotted line corresponds to the mean fluorescence value of the flat surface control. (c) Scatter plot representing mean fluorescence intensities of P. aeruginosa versus S. aureus cells attached to all topographies in the PS TopoChip.

FIG. 2—P. aeruginosa-TopoChip attachment screen analysis. (a) Binary classification tree showing that the surface parameter FCP is able to accurately categorise micro-topographies as high or low for bacterial attachment categories. (b) Bar diagram indicating the relevance of top surface parameters with predictive potential for P. aeruginosa attachment. a.u.=arbitrary units. (c) Mean fluorescence intensities from P. aeruginosa cells associated with the TopoChip TUs as a function of FCP. A Welch-t-test (with Benjamini-Hochberg correction) was applied to identify surfaces with statistically significant differences in bacterial attachment with respect to the flat surface (p<0.01).

FIG. 3A—PS TopoChip surface chemistry analysis. (a) Representative XPS spectrum, obtained from a 100×100 μm² area corresponding to a pro-attachment TU (T2-PS-1228) in a plasma-treated chip, showing F impurity originated from PS TopoChip demoulding procedure. Atomic % for C, O and F elements are shown. (b) ToF-SIMS total negative and normalized F polarity secondary ion images obtained from a 50×50 μm² area corresponding to TU 187 showing no differences in F content between topographical features of the same pattern. (c) Calculated F:C ratios from TUs with pro and anti-attachment properties against bacteria compared to flat surface control in PS TopoChip. (d) Protein depth (nm) associated to pro and anti-attachment TUs and flat control after conditioning in TSBHS10% cell culture medium for 4 h. Statistical differences between group means were determined by one-way ANOVA tests (*p<0.05).

FIG. 4—shows the attachment of bacterial cells to a surface with a microtopography applied (a) Representative images of P. aeruginosa, S. aureus, P. mirabilis and A. baumannii attachment to flat, pro and anti-attachment TUs from PS TopoChips after 4 h incubation under static conditions with planktonic bacteria. Scale bar: 50 μm. (b) Quantification of mean fluorescence intensity for P. aeruginosa, S. aureus, P. mirabilis and A. baumannii cells stained with Syto9 and grown on the same TUs from PS TopoChips.

FIG. 5—shows the attachment of bacterial cells to a surface with a microtopography applied (a) Representative images of P. aeruginosa WT stained with Syto9 fluorescent dye (green) grown on flat, pro and anti-attachment TUs from polystyrene (PS), cyclic olefin copolymer (COC) or polyurethane (PU) TopoChips. P. aeruginosa WT pME6032::mcherry (red) attachment on upside down oriented TUs (inverted) is also shown. Scale bar: 50 μm. (b) Quantification of mean fluorescence intensity of P. aeruginosa incubated under conditions described above.

FIG. 6. Early stage P. aeruginosa and S. aureus wild type and P. aeruginosa motility mutant colonization of flat, pro (T2-PS-1960) and anti-attachment (T2-PS-1307) topographies after 4 h incubation in static conditions. (a) P. aeruginosa WT, (b) S. aureus, (c) P. aeruginosa ΔpilA and (d) P. aeruginosa ΔfliC strains on flat, pro (T2-PS-1960) and anti-attachment (T2-PS-1307) topographies. Time-lapse video captures after 2 hours incubation are shown. Scale bar: 10 μm. (e) Quantification of the mean fluorescence intensity of P. aeruginosa WT, ΔpilA and ΔfliC cells attached to flat, T2-PS-1960 and T2-PS-1307 TUs after 4 h incubation under static conditions. (f) Representative images showing P. aeruginosa WT, ΔpilA and ΔfliC mutants attachment to flat, T2-PS-1960 and T2-PS-1307 TUs after 4 h incubation under static conditions. Scale bar: 50 μm.

FIG. 7—Topographical descriptors with high correlation with bacterial attachment: (A) P. aeruginosa attachment; and (B) S. aureus attachment. The topographical descriptors found to be most important for bacterial attachment are the inscribed circles which relate to the space between primitives, the average area covered by single primitives and the total area covered by primitives.

FIG. 8—Intensity maps of measured fluorescence of P. aeruginosa (a) and S. aureus (b) attached to TUs from PS TopoChips after 4-h incubation in TSBHS10% and TSB respectively. Coloured parts within the outlined area indicate the mean fluorescence intensity value for each TU (Key to right). Red frames around TUs indicate flat surface controls. (c, d) Scatter plots representing mean fluorescence intensities from P. aeruginosa (n=22) (c) and S. aureus (n=6) (d) cells attached to replicate TUs from PS TopoChips.

FIG. 9—P. aeruginosa TopoChip attachment screen analysis. (a) Mean fluorescence intensities from P. aeruginosa cells attached to the PS TopoChip TUs as a function of WN0.1. (b) Mean fluorescence intensities from P. aeruginosa cells attached to the PS TopoChip TUs as a function of WN0.5. A Welch-t-test (with Benjamini-Hochberg correction) was applied to identify surfaces with statistically significant differences in bacterial adhesion with respect to the flat surface (p<0.01). (c) Graphical display of the Pearson correlation matrix for the topographical descriptors predicting bacterial adhesion.

FIG. 10—‘Hit’ topographies with anti-attachment (more than 2.6-fold decrease compared to smooth control surface), and pro-attachment (more than 1.25-fold increase) properties (top to bottom respectively). Scale bars: 20 μm

FIG. 11—A shows graphically no change in cell viability for selected topographies identified, indicating that neither topography had a bactericidal effect on attached bacteria which could explain the differences in surface colonisation. B shows that high levels of cyclic-di-GMP are produced on flat and pro-attachment surfaces compared with ant-attachment indicative of biofilm formation on flat and pro-surfaces but not on anti-attachment surfaces

FIG. 12—shows Brightfield image sections (35 um) of flat, pro- (T2-PS-1960) and anti-attachment (T2-PS-1307) TUs showing positions (black spots) were early colonising cells of (a) P. aeruginosa wildtype, (b) P. aeruginosa ΔpilA, (d) and P. aeruginosa ΔfliC were tracked on the topographies after 3 h incubation in static conditions.

FIG. 13—Machine learning modelling results for pathogen attachment using XGBoost: (A) Scatter plot of the measured against predicted log attachment values for the P. aeruginosa test set, (B) P. aeruginosa descriptors importance, (C) Regression model performance metric results for P. aeruginosa training and test sets, and (D) Scatter plot of the standard deviation of the inscribed circles radii of the topographies; (E) Scatter plot of the measured against predicted log attachment values for the S. aureus test set, (F) S. aureus descriptors importance, (G) Regression model performance metric results for the S. aureus training and test sets, and (HJ) Scatter plot of the standard deviation of the inscribed circles radii of the topographies. The topographical descriptors found to be most important for bacterial attachment are the inscribed circle (in the figure denoted InsCircl descriptors), which relate to the space between primitives and the total area covered by primitives.

FIG. 14—High throughput screening of monocyte attachment to topographically patterned surfaces. (a) CD14+ human monocytes were isolated and cultured on polystyrene TopoChips for 3 days in the absence of any exogenous cytokines. Each data point represents the mean+/−standard deviation from 9 TopoChips tested across 5 independent donors; dotted line indicates flat planar surface Each TopoUnit was imaged independently analysed using CellProfiler to determine cell attachment the flat, planar surface had a mean attachment of 6 cells per TopoUnit indicated by blue dotted line (b) Attachment performance rank order of mean monocyte attachment (per individual TopoUnit) was calculated to compare TopoUnit performance (c) Cells were stained with a plasma membrane dye and counterstained with DAPI to quantify attachment. Representative images of high and low attachment TopoUnits (from 5 independent donors; scale bar=50 μm)

FIG. 15—Macrophage attachment is mediated by small circular pillars. (A) Macrophage attachment versus total pattern area with the size of topographical features categorised as high (circle), medium (plus/cross) or low (square) attachment. Categories of macrophage attachment were determined by cluster analysis using Euclidian distance. Representative composite confocal images of low attachment (B) and high attachment (C) TopoUnits with inset (D) orthogonal views of Z-stack images of macrophage plasma membrane (green) indicates cellular engulfment of the entire cylindrical pillar feature (also counterstained with DAPI (blue); Scale bar=10 μm).

FIG. 16—Phenotypic screening of monocyte attachment to topographically patterned surfaces. CD14+ human monocytes were isolated and cultured on plasma treated polystyrene TopoChips for 6 days in the absence of any exogenous cytokines. Each TopoUnit was imaged independently analysed using CellProfiler to determine phenotype based on mean fluorescence intensity of calprotectin (M1) and mannose receptor (M2) per cell (A) Circle chart representing the relative proportions of the macrophage phenotypic response (with SNR>2) from the TopoChip (B) Scatter plot of TopoUnit phenotype (average M2/M1 ratio) and macrophage attachment. Categories determined by hierarchical cluster analysis using Euclidian distance (phenotype represented in blue—M0, orange—M1 biased and green—M2 biased). (C-E) Representative fluorescent images of (C) M0, (D) M1 and (E) M2 biased TopoUnits with insets indicating bright field images of the topographical features. Calprotectin (M1) expression represented in yellow and mannose receptor (M2) in blue. Scale bar=20 μm.

FIG. 17—Heat map analysis of cell attachment (per donor).

FIG. 18—Machine Learning modelling results for macrophage attachment using XGBoost (a) Scatter plot of the measured against predicted values (b) SHapley Additive exPlanation (SHAP) analysis of the surface structure descriptors ranked by their average impact on model output and (c) table of results containing the random mean square error (RMSE) and R² values for the prediction model for training and test sets.

FIG. 19—Surface characterisation of TopoChip surface chemistry by 3D MS imaging and SIMS analysis. TopoChips were incubated with or without RPMI Complete Media (see SI materials and methods) for 1 hour. Comparison of peak ion intensities of the TopoChip surfaces of high (A) and low (B) macrophage attachment. Mass peaks m/z 91 (C7H7+) and 84 (C5H10N+) were used to identify the base substrate (C) and lysine as a protein marker (D), respectively.

FIG. 20—ToF SIMS positive polarity spectra acquired for media exposed TopoUnits compared for high (Black) and low (Red) attachment (m/z 0-1000).

FIG. 21—XPS elemental analysis of TopoUnit surfaces. (a) Representative XPS spectrum, obtained from a 100×100 μm² area corresponding to a flat planar plasma-treated surface. Elemental composition for C, O and N elements are shown (atomic %). (b) Protein layer thickness on a range of high (white bars) and low attachment (grey bars) TopoUnit surfaces compared to the flat area (black bar). Data expressed as protein layer thickness+/−standard deviation of at least three independent samples.

FIG. 22—ToF MS images of relative total ion distribution, CNO⁻ and CNO-normalised to total ion count; images show regions of untreated and 1 hr treated on (A) high and (B) low attachment TopoUnits, respectively. Images representative of three independent areas analysed per sample. Data acquired with 30 keV Bi3+(lateral resolution ˜2 μm, pixel size 2 μm)

FIG. 23—Machine Learning modelling results for macrophage polarisation using XGBoost (a) Scatter plot of the measured against predicted values (b) SHapley Additive exPlanation (SHAP) analysis of the surface structure descriptors ranked by their average impact on model output and (c) table of results containing the random mean square error (RMSE) and R² values for the prediction model for training and test sets.

FIG. 24—TopoUnit descriptors of important surface features compared to the composite variable “Log(M2/M1)×attachment” to represent phenotype importance in high attachment (□ and • represent the top 50 M1 and M2 performing TopoUnits, respectively). Statistical significance of P<0.05 determined using Mann-Whitney test.

FIG. 25—Topographies which significantly slow down DC motility and reduce length of travel in comparison to the on-chip flat surface control. Mean speed per Track from each topographic pattern, arranged in a ranked order from fast to slow. Time-lapse videos were analysed and speed per cell was averaged per topographic unit. n=10 from 6 donors. Data presented as mean±SD. Significantly (p<0.001) changed motilities have been highlighted.

FIG. 26—Topographies which significantly slow down DC motility and reduce length of travel in comparison to the on-chip flat surface control. Track displacement from each topographic pattern, arranged in a ranked order from furthest movement to least movement. Time-lapse videos were analysed and cell displacement was averaged per topographic unit. n=10 from 6 donors. Data presented as mean±SD. Significantly (p<0.001) changed motilities have been highlighted.

FIG. 27—shows a strong correlation between mean speed and track displacement of cells on topographies.

FIG. 28—shows the five fastest and five slowest motility generating topographic features—ranked from fast to slow.

FIG. 29—shows the five topographic features that support the most movement and five that support least cell movement—ranked from furthest to least movement.

FIG. 30—shows the correlation mean speed of DCs on topographies and the diagonal spacing in between features.

FIG. 31—Examples of feature spacing classes

FIG. 32—Modulation of HLA-DR expression on immature DCs following 24 hour culture on topographies. Cells were stained for HLA-DR and analysed via flow cytometry. Data is from 3 independent experiments and shows MFI and percentage of positive cells as mean±SD. Experimental conditions are compared to the flat surface control to compare fold changes over different donors. For significance: *<0.0332; **<0.0021; ***<0.0002; ****<0.0001, by Students T-Test.

FIG. 33—Modulation of PD-11 expression on immature DCs following 24 hours culture on topographies. Cells were stained for flow cytometry. Data is from 5 independent donors and shows MFI, percentage of positive cells as mean SD. Experimental conditions are compared to the flat surface control (normalised) in order to compare trends over different donors.

FIG. 34—Modulation of CCR7 expression on immature DCs following 24 hours culture on topographies. Cells were stained for flow cytometry. Data is from 5 independent donors and shows MFI, percentage of positive cells as mean SD. Experimental conditions are compared to the flat surface control (normalised) in order to compare trends over different donors.

FIG. 35—Modulation of CD86 expression on immature DCs following 24 hours culture on topographies. Cells were stained for flow cytometry. Data is from 5 independent donors and shows MFI, percentage of positive cells as mean SD. Experimental conditions are compared to the flat surface control (normalised) in order to compare trends over different donors.

FIG. 36—Modulation of CD83 expression on immature DCs following 24 hours culture on topographies. Cells were stained for flow cytometry. Data is from 5 independent donors and shows MFI, percentage of positive cells as mean SD. Experimental conditions are compared to the flat surface control (normalised) in order to compare trends over different donors.

FIG. 37—Modulation of HLA-DR expression when DCs were stimulated with LPS on topographies and cultured for 24 hours. Cells were stained for HLA-DR and analysed via flow cytometry. Data is from 3 independent experiments and shows MFI and percentage of positive cells as mean±SD. Experimental conditions are compared to the flat surface control to compare fold changes over different donors. For significance: *<0.0332; **<0.0021; ***<0.0002; ****<0.0001, by Students T-Test.

FIG. 38—Modulation of CCR7 expression when DCs were stimulated with LPS on topographies and cultured for 24 hours. Cells were stained for CCR7 and analysed via flow cytometry. Data is from 3 independent experiments and shows MFI and percentage of positive cells as mean±SD. Experimental conditions are compared to the flat surface control to compare fold changes over different donors. For significance: *<0.0332; **<0.0021; ***<0.0002; ****<0.0001, by Students T-Test.

FIG. 39—Modulation of CD86 expression when DCs were stimulated with LPS on topographies and cultured for 24 hours. Cells were stained for CD86 and analysed via flow cytometry. Data is from 3 independent experiments and shows MFI and percentage of positive cells as mean±SD. Experimental conditions are compared to the flat surface control to compare fold changes over different donors. For significance: *<0.0332; **<0.0021; ***<0.0002; ****<0.0001, by Students T-Test.

FIG. 40—Modulation of PD-L1 expression when DCs were stimulated with LPS on topographies and cultured for 24 hours. Data is from 3 independent experiments and shows MFI and percentage of positive cells as mean±SD. Experimental conditions are compared to the flat surface control to compare fold changes over different donors.

FIG. 41—Modulation of CD83 expression when DCs were stimulated with LPS on topographies and cultured for 24 hours. Data is from 3 independent experiments and shows MFI and percentage of positive cells as mean±SD. Experimental conditions are compared to the flat surface control to compare fold changes over different donors. For significance: *<0.0332; **<0.0021; ***<0.0002; ****<0.0001, by Students T-Test.

FIG. 42—Assessing modulated cytokine secretion of IL-10 after culturing iDCs (A,C) or with LPS stimulated DCs (B,D) for 24 hours on topographies. Topographies are investigated for their ability to modulate IL-10 secretion. Experimental conditions are compared to the flat surface control. Data is from 3 independent experiments and represented as mean±SD and fold change normalised to flat control. ONE-way ANOVA with Bonferroni multiple comparisons test. For significance: *<0.0332; **<0.0021; ***<0.0002; ****<0.0001.

FIG. 43—Assessing modulated cytokine secretion of IL-12 after culturing iDCs (A,C) or with LPS stimulated DCs (B,D) for 24 hours on topographies. Topographies are investigated for their ability to modulate IL-12 secretion. Experimental conditions are compared to the flat surface control. Data is from 3 independent experiments and represented as mean±SD and fold change normalised to flat control. ONE-way ANOVA with Bonferroni multiple comparisons test. For significance: *<0.0332; **<0.0021; ***<0.0002; ****<0.0001.

FIG. 44—Pan T cell proliferation for topography-modulated DCs. Proliferation was assessed by BrdU incorporation and then calculated into a stimulation index. n=1, N=2.

FIG. 45—shows that cytokine production may be affected upon 8 days of co-culture with T-cells and DCs on specific microtopographies. 384 well plates were used to test (a) IFN gamma production, (b) IL-17 production or (c) IL-10 production after 8 days of co-culture, using ELISA.

FIG. 46—A shows the use of 3 primitive shapes—namely a rectangle, triangle and circle that are combined to form a micropillar structure. Typically the micropillars are shaped according to a topography determined using a screening technique of possible primitive shape combinations. B shows a mathematical image of micropillars (circled in yellow) that together form a feature (red) that is repeated across the surface. C shows the micropillar features together form a, in a brightfield image of such a topochip. The feature is shown in the box (Red). In the example shown this is a 290×290 μm. D shows the effect of the features on the surface is to consider an inscribed circle analysis of the descriptors. This is shown in where circles are used to define space between the micropillars that form the feature and also to adjacent features.

FIG. 47 shows non-linear regression for the bacteria P. aeruginosa. A shows results for Random Forest Modelling with Top 8 Important Features selected by SHAP for the Test Set. In particular, the graph of B shows the important features, ranked from most to least important. SHAP scale shows negative/positive influence on the outcome. For instance, High values of TotalArea (pink) are more likely to have negative effect on Attachment. Mid range values for TotalArea can have both positive or negative (purple values); low values (blue) have positive effect on Attachment.

FIG. 48—shows the effect of each variable on the average fluorescence (i.e. attachment).

FIG. 49 is a model with Pattern Area Max, InscrCirclesMean and InscrCirclesSD produces R2=0.71 in the model. So we can build good models without total area. Pattern Area Max is the area of the bigger pillar in a feature. FIG. 49 shows the graph of FIG. 48 converted to microns.

FIG. 50—shows the effect of attachment of the bacteria when analysed to topochips using the inscribed circles technique to define coverage. Strong anti-attachment topography cannot have radius mean≥3.2 px (0.32 μm) and SD≥1.4 px (0.14 μm).

FIG. 51—shows the attachment compared to the max patterned area. Where the max patterned area is the area of the biggest pillar in the feature. Here Medium/low-attachment≥170 μm2; High Attachment<40 μm2, but other factors need to be considered, such as the space between the pillars.

FIG. 52—shows the results for the bacteria S aureus. As shown in FIG. 52, Results for Random Forest with Top 8 Important Features selected by SHAP for the Test Set. The graph shows the important features, ranked from most to least important. SHAP scale shows negative/positive influence on the outcome. For instance, High values of TotalArea (pink) are more likely to have negative effect on Attachment. Some mid range area topographies also affect negatively attachment. Mid range values for TotalArea can have both positive or negative (purple values); low values (blue) have positive effect on Attachment.

FIG. 53—outlines the important descriptors that can be used for attachment of S. aureus.

FIG. 54—shows the attachment in relation to the total area can derive a design rule for the descriptor micropillars of Pro-attachment≤500 μm2; Anti-attachment≥1350 μm2.

FIG. 55—shows that for inscribed circles Strong anti-attachment topography cannot have radius mean>0.3 μm and SD>0.1 μm.

FIG. 56—shows attachment in relation to max pattern area, where Medium/low-attachment≥280 μm2 Or less than 30 μm2 depending on values for other descriptors.

FIG. 57 shows high Attachment Topography Examples The black elements are the featured topographies; the blue circles are the inscribed circles one can fit between the topographies.

FIG. 58—shows low attachment topography examples.

FIG. 59—The number of P. aeruginosa and S. aureus cells captured in the bulk medium in 40 μm image stacks above the surface of flat, pro- (T2-PS-1960) and anti-attachment (T2-PS-1307) TUs after 3 hours exposure in static conditions. Data shown are mean±SD, n=3. Statistical analysis was done using a two-way ANOVA with Dunnett's multiple comparisons test (*p<0.05; **p<0.01; ***p<0.001; ****p<0.0001).

### METHODS

TopoChip Design

The TopoChip was designed by selecting 2176 features from a vast in silico library of features containing a single or multiple 10 μm high pillars within an imaginary square of either 10 by 10, 20 by 20, or 28 by 28 μm² size (Unadkat et al., 2011). Micro-pillars were built up using three types of microscale primitive shapes: circles, triangles, and rectangles (3 μm width). Topographies were assembled as periodical repetitions of the features within 300 μm×300 μm micro-wells surrounded by 40 μm tall walls (TopoUnits—TUs) in a 66 by 66 array containing a duplicate TU for each topography and flat control surfaces. TopoChips were fabricated on a 2×2 cm² chip as previously described (Unadkat et al., 2011; Zhao et al., 2017). Briefly, the inverse structure of the topographies was produced in silicon by standard photo lithography and deep reactive etching. The silicon mould was used to make a positive mould in poly(dimethylsiloxane) (PDMS). The PDMS mould was required to create a second negative mould in OrmoStamp hybrid polymer (micro resist technology Gmbh), which served as the mould for hot embossing polystyrene (PS), polyurethane (PU) and Cyclic olefin copolymer (COC) films (Goodfellow) to make the TopoChips. After fabrication the arrays were subjected to oxygen plasma etching to reduce the hydrophobicity of the material.

Surface Chemistry Analysis

The surface chemistry of the TopoChip was assessed using time-of-flight secondary ion mass spectrometry (ToF-SIMS) and X-ray photoelectron spectroscopy (XPS). ToF-SIMS measurements were conducted on an ION-ToF IV instrument using a monoisotopic Bi₃⁺ primary ion source operated at 25 kV and in ‘bunched mode’. A 1-pA primary ion beam was rastered, and both positive and negative secondary ions were collected from 0.05 mm², 0.1 mm², 0.4 mm² or 10 mm² areas. Ion masses were determined using a high resolution Time-of-Flight analyser.

The chemistry of the TopoChip surfaces were quantified in terms of elemental composition using an Axis-Ultra XPS instrument (Kratos Analytical, UK) with a monochromated A1 kα X-ray source (1486.6 eV) operated at 10 mA emission current and 12 kV anode potential (120 W). Small spot aperture mode was used in magnetic lens mode (FoV2) to measure a sample area of approximately 110 μm². A wide scan at low resolution (1400 to −5 eV binding energy range, pass energy 80 eV, step 0.5 eV, sweep time 20 minutes) was used to estimate the total atomic % of the detected elements. High resolution spectra at pass energy 160 eV with step of 1.0 eV and sweep time of 20 min were also acquired for photoelectron peaks from the detected elements and these were used to model the chemical composition. CasaXPS (version 2.3.18dev1.0x) software was used for quantification and spectral modelling.

The measured N is fraction in medium conditioned surfaces was converted into protein layer thickness using Ray & Shard (2011) relationship between [N] and protein depth.

Bacterial Strains and Culture Conditions

The pathogens P. aeruginosa PAO1, Staphylococcus aureus SH1000, Proteus mirabilis Hauser 1885 and Acinetobacter baumannii ATCC17978 used in this work were routinely grown at 37° C. on lysogeny broth (LB) or LB agar supplemented with antibiotics as required. These species were selected as representatives of both Gram-negative (P. aeruginosa, P. mirabilis, A. baumannii) and Gram-positive (S. aureus) pathogens commonly associated with medical device infections (Percival et al., 2015). Tryptic soy broth (TSB) medium was used to study bacterial attachment to the TopoChip. To mimic in vivo conditions and stimulate P. aeruginosa PAO1 attachment to the TopoChip, TSB supplemented with 10% human serum (TSBHS10%) was used. For PU TopoChip attachment studies, P. aeruginosa PAO1 carrying the constitutively expressed mcherry gene on the plasmid pMMR (Popat et al., 2012) was used. P. aeruginosa PAO1 flagellum and type IV pili (TFP) deficient strains, ΔfliC and ΔpilA respectively, were included in this study to assess the influence of bacterial appendages on attachment to micro-topographies.

Prior to incubation with bacteria, TopoChips were washed by dipping in distilled water followed by sterilisation in ethanol. Air-dried micro-topographical arrays were then placed horizontally in petri dishes (60 mm×13 mm) and incubated statically or with shaking (60 rpm) at 37° C. in 10 ml of medium inoculated with diluted (OD₆₀₀=0.01) bacteria from overnight cultures. It should be noted that despite air bubbles formed on the arrays surface after media immersion, air pockets were straightforwardly detached by brief incubation at 37° C. and repeated media pipetting. Hence, no entrapped air between the topographical features could be detected in any experiment carried out in this study. At the desired time point, TopoChips were removed and washed by dipping 5 times in 25 ml of PBS to remove loosely attached cells. After rinsing with distilled water to remove salts, attached cells were stained with 50 μM Syto9 (Molecular Probes, Life Technologies) for 30 min at room temperature. Following staining, chips were rinsed with distilled water, air-dried and mounted on a glass slide using Prolong antifade reagent (Life Technologies). Viability of attached cells was evaluated by fluorescent staining with the LIVE/DEAD® BacLight™ Bacterial Viability kit (Molecular Probes, Life Technologies) following the manufacturer's instructions.

TopoChip Imaging and Data Acquisition for Bacterial Attachment

TopoChips were imaged using a Zeiss Axio Observer Z1 microscope (Carl Zeiss, Germany) equipped with a Hamamatsu Flash 4.0 CMOS camera and a motorized stage for automated acquisition. A total of 4356 images (one per TU) were acquired for each chip using a 488 nm laser as light source. A magnification lens (Zeiss, EC Plan-Neofluar 10×/0.30 Ph 1) was used to provide enough depth resolution to capture the total fluorescence per TU. Images were cropped to a 247 μm×247 μm field of view so that the walls of the micro-wells were not included in the image, to reduce the occurrence of artefacts due to bacterial attachment to walls. To identify out-of-focus images from each TopoChip dataset, individual topography images were combined into composites using open source Fiji-ImageJ 2.0.0 software (National Institutes of Health, US). Montages were then visually inspected and blurry images excluded from data analysis. Image pre-processing included a) staining artefact removal by excluding pixels with fluorescence intensities higher than 63,000 from data acquisition and b) background removal using “Gaussian blur” (σ=2) image filtering and “Rolling Ball Background Subtraction” (radius=15) tools from Fiji-ImageJ software. Due to the dimmer fluorescent staining of attached P. aeruginosa cells, background correction was further enhanced for this bacterium by removing TU-specific autofluorescence from Polystyrene TopoChips. TU-specific noise was determined from PS TopoChips (n=3) incubated without bacteria and used for data normalization.

To classify topographies that had a positive or negative effect on bacterial attachment, the fluorescence signal from each topography was used to quantify the amount of bacterial attachment. The mean fluorescence intensity on each TU was measured using Fiji-ImageJ and each value was normalized to the average fluorescence intensity of the chip to account for differences in staining intensities between experiments. Hit micro-topographies with anti-attachment properties were selected for further studies based on the screening data obtained from quantifying P. aeruginosa (n=22) and S. aureus (n=6) attachment to PS TopoChips. Antifouling TUs with more than 2.6-fold reduction in the mean fluorescence intensity of flat control for P. aeruginosa and S. aureus were designated. Similarly, TUs with 1.25-fold or more increase of P. aeruginosa were chosen as pro-attachment micro-topographies and used in additional studies. Welch-t-test with Benjamini-Hochberg multiple testing correction was applied to determine whether bacterial attachment on TUs differed significantly from that of flat control (p<0.01) as compared to the variations within the replicates. Welch-t-test was selected to account for unequal variances and sample sizes, while the Benjamini-Hochberg correction procedure was necessary to calculate and adjust the p-value (typically increased) to reduce the number of false positives since the Welch-t-test is repeated multiple times to pairwise test every TU versus flat.

For single-cell tracking experiments an inverted TE2000 microscope (Nikon, Japan) equipped with a Hamamatsu Orca Flash 4.0v2 sCMOS camera and an OKOLab cage incubation system to maintain temperature (37° C.) and humidity constant (95%) was used. Time series z-stacks (50 μm range-2 μm steps) were acquired every min for 4 h from selected TUs using a 40× objective (Nikon, CFI Plan Fluor 40×Oil/1.3).

To track motile bacterial cells on the surfaces, z-stacks were processed in MATLAB R2015a (MathWorks) by subtracting images outside the focal plane and establishing a manual threshold to identify pixels representing bacterial cells. Then the ellipse-fitting method was applied to obtain the centre-of-mass of the objects and a custom designed script was used to build trajectories from single-cell positions. To minimize tracking errors, images from early bacterial colonisation were used (<4 h) to avoid crowded surfaces and cell trajectories generated were validated by visually inspecting cell displacement. The instantaneous and average speeds of bacterial surface-associated movements were calculated using equations (1) and (2), where Δt=tᵢ₊₁−tᵢ and n is the number of points in the trajectory. Motile bacteria were defined as cells travelling with a minimum speed of 5 nm sec⁻¹. Due to the feature sets with narrower spacing in anti-attachment TUs, it was not possible to identify cell trajectories in this surfaces.

$${\vec{v}}_{i} = \vert \vec{x} ( {t}_{i + 1} ) - \vec{x} ( {t}_{i} ) \vert / Δ t \qquad ( 1 )$$

$${\vec{v}}_{average} = \frac{1}{n} {\sum }_{i = 1}^{n} ( {\vec{v}}_{i} ) \qquad ( 2 )$$

To further characterize bacterial behaviour on micro-topographies, bacterial displacement on selected TUs was described by assessing the sum of trajectory distances from mean (SD) and mean squared displacement (MSD). Both parameters give information about the average displacement between points in a trajectory separated by a fixed time interval. SD was calculated as in equation (3), where \vec{R}ᵢ is the position vector of the i^{th} point and \vec{R}_{cm} is the centre-of-mass of all points. MSD was estimated using equation (4) (Utada et al., 2014), where \vec{x}(tⱼ) is the vector of the j^{th} point on the trajectory and angled brackets represent average over all times tᵢ.

$${\vec{SD}}_{cm} = \frac{1}{n} {\sum }_{i = 1}^{n} ( ( {\vec{R}}_{i} ) - ( {\vec{R}}_{cm} ) ) \qquad ( 3 )$$

$$Δ {x}^{2} ( t ) = 〈 {( \vec{x} ( {t}_{i + 1} ) - \vec{x} ( {t}_{i} ) )}^{2} 〉 \qquad ( 4 )$$

Topography Form-Bacterial Attachment Analysis

The bacterial attachment fluorescence value for each the replicate topounits data were averaged for a number of chips to calculate and standard deviations were calculated. The fluorescence intensity is established to correlate with the number of attached fluorescent bacteria as has been shown previously (Hook et al Nat. Biotech. 2012). It was therefore used as the dependent variable in the models. Topounits with low signal to noise ratio (<2) were excluded from the datasets of P aeruginosa (342 units removed) and S aureus attachment (93 units removed). The XGBoost machine learning method (Chen and Guestrin, 2016) was applied to generate relationships between the topographies and bacterial attachment using the topographical descriptors listed in Supplementary Table S1. The XGBoost module was used with default parameters in Python 3.7. Seventy percent of each bacterial attachment dataset was used to train the model, and 30% were kept aside to determine the predictive power of the model. The SHAP (SHapley Additive exPlanations) package in Python 3.7 (Lundberg and Li, 2017) was used to eliminate less informative descriptors and to determine descriptor importance.

The 2176 unique micro-topographies on the TopoChip were labelled as follows: T2-XX-aabb, where T2 indicates the version of the TopoChip design, XX the substrate material (e.g. PS), aa the array row number (ranging from 01 to 33) and bb the column number (ranging from 01 to 66). Importantly, the flat surface control topography was positioned in the bottom right corner prior TopoChip imaging, to allow consistent numbering of the TopoUnits.

Murine Foreign Body Infection Model

To investigate the progress of bacterial infection and the host immune response to pro-(T2-PU-1228, T2-PU-2056) and anti-(T2-PU-0709, T2-PU-1307, T2-PU-1429 and T2-PU-2153) attachment TUs, a murine foreign body infection model was used (Hook et al, 2012). TUs (3 mm×7 mm) with micro-patterns imprinted on one side were fabricated in PU. Using a 9 gauge trocar needle, rectangular sections of scaled up PU-TUs were implanted subcutaneously (1 per mouse, 3 repeats for each micropattern topography), into 19-22 g female BALB/c mice (Charles River) with the patterned side facing upwards to the skin surface. One hour before implantation, 2.5 mg/kg of Rymadil analgesic (Pfizer) was administered by subcutaneous injection. Animals were anaesthetized using isoflurane, the hair on one flank removed by shaving and the area sterilized with Hydrex Clear (Ecolab). After foreign body insertion, mice were allowed to recover for 4 days prior to injection of either 1×10⁵ colony forming units (CFUs) of P. aeruginosa or vehicle (phosphate buffered saline; uninfected control).

Mice were housed in individually ventilated cages under a 12 h light cycle, with food and water ad libitum, and with weight and clinical condition of the animals recorded daily. Four days post infection, the mice were humanely killed and the micropatterned PU TU samples and the surrounding tissues removed. PU TU samples were fixed in 10% v/v formal saline and labelled with antibodies targeting CD45 (pan-leukocyte marker; VWR violetfluor 450), CD206 (macrophage mannose receptor; Biorad rat anti-mouse antibody conjugated to Alexa 647) and the membrane-selective dye FM1-43 (Thermofisher Scientific) for total TU-associated biomass. Bacterial cells on the micropatterned surfaces were visualized with polyclonal antibodies raised against P. aeruginosa (Thermofisher Scientific). Secondary antibodies used were goat anti-rabbit (quantum dot 705; Thermofisher). Images were taken on a Zeiss 700 confocal microscope and fluorescence data quantified using Image J. All animal work was approved following local ethical review at University of Nottingham and performed under U.K. Home Office Project Licence 30/3082.

Monocyte Isolation

Buffy coats form healthy donors were obtained from the National Blood Service (National Blood Service, Sheffield, UK) following ethics committee approval (2009/D055). Peripheral blood mononuclear cells (PBMCs) were isolated from heparinised blood by Histopaque-1077 (Sigma-Aldrich) density gradient centrifugation. Monocytes were isolated from PBMCs using the MACS magnetic cell separation system (positive selection with CD14 MicroBeads and LS columns, Miltenyi Biotec) as described previously (18. May R M, Hoffman M G, Sogo M J, Parker A E, O'Toole G A, Brennan A B, Reddy S T. Micro-patterned surfaces reduce bacterial colonization and biofilm formation in vitro: Potential for enhancing endotracheal tube designs. Clin Transl Med 2014; 3:8)

Monocyte Culture

Purified monocytes were suspended in RPMI-1640 medium supplemented with 10% foetal bovine serum (FBS), 2 mM L-glutamine, 100 U/ml penicillin, and 100 μg/ml streptomycin (all from Sigma-Aldrich) (henceforth referred to as “complete medium”) and seeded at 3×10⁶ cells/well in 6-well polystyrene plates (Corning Life Sciences).

TopoChip Imaging and Data Acquisition for Monocyte Adhesion

All samples were inverted, and fluorescent images acquired using a Zeiss Axio Observer Z1 microscope (Carl Zeiss, Germany) equipped with a Hamamatsu Flash 4.0 CMOS camera and a motorized stage for automated acquisition. A Zeiss, EC Plan-Neofluar 20×/0.50 Ph 2) was used to provide sufficient resolution to capture the fluorescence data whilst enabling the use of the auto-focus function, which considerably reduced scanning times and file sizes per TopoChip. Images were cropped to a smaller field of view (280 μm×280 μm) that did not include the walls of the TopoUnits to improve the auto-focus function. Following acquisition, images were manually inspected to identify out of focus images which were removed from the analysis. Subsequently, all individual TopoChip images were analysed using open source software Cell Profiler (CP) using custom made pipelines. After illumination corrections, nuclei were detected as the primary objects using the Robust Background thresholding method applied globally on the DAPI channel. Subsequently, cell demarcation and morphology were determined by applying a Watershed gradient method with background thresholding applied and appropriate propagation algorithms on the CellMask (plasma membrane) channel. Cells found to be in contact with the edges were filtered out of the dataset. For cell phenotyping analysis, the mean fluorescent intensity value inside the segmented cell area is summarized to determine the value for each respective fluorescent channel.

Computational Analysis for Monocyte Adhesion

To identify the surface design parameters that can influence monocyte adhesion, monocyte attachment screening data of CD14+ human monocytes on 30 second plasma treated polystyrene TopoChips were studied. Data was first pre-processed and, for each donor, the values quantifying mean fluorescence of Calprotectin, MR and the total cell count per topography were normalised by their corresponding flat topography values. As cell fluorescence and attachment may be heterogeneous due to poor representation on the slide, replicates by donor were averaged, and those TopoUnits with signal to noise ratio (SNR) lower than two were excluded from the analysis for most cases. There were circumstances of low attachment, however, where the SNR values were carefully moderated by the standard deviation values. Subsequently, average, standard deviation and signal to noise ratio (SNR) were calculated between donors for the modelling studies.

As attachment and polarisation were both important, machine learning models were trained to predict initially attachment; subsequently, a composite dependent variable Log(M2/M1)×Attachment to investigate attachment associated with polarisation properties was investigated. The TopoUnit topographies are computationally described using a combination of surface feature parameter values used to construct the features in addition to Cell Profiler generated parameters (from bright field images) which describe characteristics of surface feature area and shape. A total of 246 descriptors was investigated. SHapley Additive exPlanation (SHAP) method was employed for feature selection to eliminate uninformative and less informative descriptors. SHAP was implemented using the shap package in Python 3.7.^{[35]} Regression models were generated using random forest and XGBoost, using the packages sklearn and xgboost in Python 3.7. 70% of the data instances were employed for model training and 30% for testing.^{[36, 37]} The performance of the predictive models and the topographical descriptors that contributed most strongly to the attachment and polarisation were consistent for both methods. Results for XGBoost are shown in Figure S2 & 5. The figure presents the results of the regression models as well as the features selected. The features are ordered from top to bottom based on their average impact on the model output magnitude.

Feature Descriptor Generation for Monocytes

In addition to topography design descriptors that were extracted directly from the design file and were reported elsewhere, we have obtained an additional set of features as following: topographies designs were represented as black and white (binary) images where white corresponded to the design of the pillars and black to the spacing between them.[21] Images were created from the design file of the topographies in custom Matlab 2017 script. Only images of unique topographical features and spacing around them (Feature Block) were used. 10 pixels on the resulted images corresponded to the 1 um on real fabricated surfaces. Shape and Size related Surface Descriptors were extracted via custom build image analysis pipeline constructed in CellProfiler 2.2.

For quantification of the spacing between pillars Feature Block binary images were inverted and replicated across the area that corresponds to real fabricated surfaces. To reduce the size of the resulted images they were downscaled 5 times. We further employed MaxInscribed Circles algorithm as described here https://imagej.net/Max_Inscribed_Circles. To identify the size and number of circles that can be fitted in the gap between pillars. The algorithm is looped until a circle diameter smaller than 3 pixels is found.

Further analysis was performed in R version 3.5 unless specified differently. Per topography, summary statistics of topography design descriptors such as mean, median, percentile, number of pillars, was quantified. This generated a library of 246 topographical descriptors, subsequently, Pearson correlation analysis was applied to remove overlapping and non-intuitive descriptors (≥0.85). This finalised descriptor library of 65 physical surface determinants was used for modelling and correlative analysis.

Immunocytochemistry for Monocyte Studies

For attachment experiments: Cells were fixed with 4% paraformaldehyde (Bio-Rad) in PBS as described above, washed thrice with PBS (5 min per wash), then permeabilized by 0.2% Triton-X100 (Sigma-Aldrich) in PBS for 20 min. After 3 washes with PBS, non-specific binding was blocked with 5% goat serum in PBS as described in the previous section. This was followed by 2 washes with PBS and incubation with the cellular stain, CellMask™ (Invitrogen) in PBS for 30 min. Cells were then washed 3 times with PBS and stained with 250 ng/ml DAPI (4′, 6-Diamidino-2-Phenylindole) (Invitrogen) in PBS for 5 min, washed 3 times with PBS, then mounted with anti-fade medium (Pro-Long Gold), and on a standard microscope slide followed by imaging using a fluorescent microscope (Zeiss).

For phenotypic analysis: Adherent cells on coverslips were fixed with 4% paraformaldehyde (Bio-Rad) in PBS for 10 min. Fixation and all subsequent steps in this procedure were carried out at room temperature; all washes were carried out with 0.2% Tween 10 (Sigma-Aldrich) in PBS (5 min per wash) except where stated. Following fixation, cells were washed three times, then blocked with 1% (w/v) glycine (Fisher Scientific) and 3% (v/v) bovine serum albumin (BSA, Sigma-Aldrich) in PBS for 30 min. Subsequently, cells were washed twice and incubated for 30 min with 5% (v/v) goat serum (Sigma-Aldrich) in PBS to block non-specific antibody binding. Next, cells were incubated for 1 h with the appropriate primary, washed 3 times, and then incubated for 1 h with the appropriate secondary antibody at room temperature. Finally, all cells were stained with 250 ng/ml DAPI (4′,6-Diamidino-2-Phenylindole) (Life Technologies) in PBS for 5 min, washed 3 times with PBS, then mounted with anti-fade medium (Pro-Long Gold), on a standard microscope slide followed by imaging using an automated fluorescent microscope (Zeiss).

DC Cell Culture

DC were cultured at 1×10⁶ DCs/mL for 24 hours on the topographies. 1 repeat of the topographies was stimulated with 10 ng/mL LPS after 6 hours of DC conditioning on the topographies.

DC Staining and Live Cell Imaging

Dendritic cells were stained with Hoechst nuclear stain (400 ng/mL) for 20 minutes as well as CFSE cytoplasmic stain for 15 minutes in serum-free media, followed by 30 minutes incubation to induce CFSE hydrolysis. Cells were seeded at 1×10⁶ cells/mL onto a uniform chemistry topography chip and left to settle for 30 minutes before live imaging started. Different positions of interest were programmed into the ROI manager, so the view field will cover the entire 350 um of the squared topography unit at 4× magnification. To cover all 36 positions (including a flat control) the interval of images was set to 7 minutes and set to record for 3 hours in total. Images were acquired in brightfield, Hoechst and CFSE channels on a DeltaVision set up. Cells were kept at 37 C and 5% CO₂ throughout the entire imaging process.

DC Flow Cytometry Staining

To assess modulation of surface maker expression DCs were harvested, washed with PBA, and stained with CD83-FITC, CD86-PE, PD-L1 APC, CCR7-PE-Cy7 and HLA-DR PerCP antibodies for 20 minutes. Cells were then again washed with PBA, fixed in 1% PFA and acquired on a Canto flow cytometer.

DC ELISA

Cell culture supernatant was harvested after 24 hours of cell culture and stored at −20 C until further use. IL-10, IL-12p70, IFNgamma and IL-17 assays were run in a 384-wellplate (R&D Systems).

DC Co-Culture with T-Cells

DCs were co-cultured in 1:10 with Pan T cells in human serum supplemented complete media for 8 days. On day 3 100 uL of the cell culture was removed and substituted with fresh media supplemented with 5 ng/mL IL-2. On day 7 the positive controls were stimulated with 2 ug/mL anti-CD3 and 2 ug/mL anti-CD28 monoclonal antibodies (Sigma Aldridge). After 8 days, the cell culture supernatant was harvested and stored at −20 C until further use.

DC Study—T-Cell Proliferation Assay

Day 7 of the DC-Pan T cell co cultures 20 uL of prepared Bromodeoxyuridine (BrdU)-labelling solution was added into the wells. BrdU is a synthetic nucleoside and an analog for thymidine. During the S phase of the cell cycle (when the DNA is replicated) BrdU will incorporate itself into the newly synthesized DNA of replicating cells instead of thymidine. BrdU-specific antibodies can then be used to detect and quantify the level of incorporation.

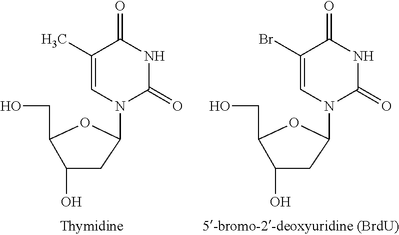 [SMILES: Cc1cn(C2CC(O)C(CO)O2)c(=O)[nH]c1=O ; O=c1[nH]c(=O)n(C2CC(O)C(CO)O2)cc1Br]

The wellplates were dried in the oven for 1 hour at 60 C, after which they can be stored for up to 1 week in the fridge. In order for the BrdU-antibody to be able to bind to the incorporated BrdU the DNA has to be denatured by heat or acid (in this case fixative/denaturation solution consisting of acid). After denaturation of the DNA the anti-BrdU antibody can bind to the BrdU incorporated in the DNA. The anti-BrdU antibody which was used here is conjugated to peroxidase (POD). Following washing steps and addition of the colourless substrate solution (TMB/peroxide) which the peroxidase conjugated to the antibody will catalyse to a colour change. To stop this colorimetric reaction H2SO4 (1M) is added and absorbance levels measured at 450 nm and for reference at 600 nm.

DC Studies—Statistical Analysis

Data were analysed using GraphPad Prism version 8.01; results are presented as mean±SD. Statistical differences between the experimental conditions were determined using one-way ANOVA with Dunnett's multiple comparisons as indicated. A level of p<0.05 was considered statistically significant.

TABLE 1
Micro-topographies surface descriptors

| Surface property | Description |
|---|---|
| FeatSize | The size of the bounding square for the |
| | primitives (10, 20 or 28 μm) |
| NumCirc | The number of circles used |
| NumTri | The number of triangles used |
| NumLine | The number of lines used |
| CircDiam | Circle diameter |
| TriSize | Length of the shortest side of a triangle |
| LineLen | Line length |
| RotSD | The standard deviation (in degrees), is used |
| | to determine the rotation of the primitives |
| | when they are placed in the feature |
| Rot | The standard deviation for rotation of primitives |
| | scaled with number of line and triangle primitives |
| | (since circle primitives are unaffected by rotation) |
| WN0.1-WN4 | The fraction of energy in the signal in sinusoids |
| | with wave number 0.1-4 |
| CircArea | The area of circle primitives |
| TriArea | The area of triangle primitives |
| LineArea | The area of line primitives |
| DC | The number of circle primitives scaled by |
| | feature area |
| DT | The number of triangle primitives scaled by |
| | feature area |
| DL | The number of line primitives scaled by |
| | feature area |
| CA | The total area of circle primitives scaled by |
| | feature area |
| TA | The total area of triangle primitives scaled by |
| | feature area |
| LA | The total area of line primitives scaled by |
| | feature area |
| CCD | Number of colour changes of the feature over |
| | the diagonal |
| FCP | Number of pixels covered by primitives divided |
| | by the total number of pixels |
| FCPLOG | ln(FCP/1-FCP) |
| FCPN0.1-0.3 | FCP Nx = max(min(FCP + ϵ, 0.99), 0.01) |
| | ϵ: normally distributed noise with mean 0 and |
| | standard deviation x |
| FCPLOGN0.1-0.3 | ln(FCP Nx/1-FCP Nx) |

Feature refers to the bounding square of 10, 20 or 28 μm including micro-pillars and space between them (see FeatSize). Each micro-topographical element contains primitives (circles, triangles and rectangles). Features are repeated to cover the surface of a TopoUnit.

SUPPLEMENTARY TABLE 1
TopoUnit topography surface descriptors

| Surface property | Description |
|---|---|
| NumTri | The number of triangles used |
| NumLine | The number of lines used |
| CircDiam | Circle diameter |
| TriSize | Length of the shortest side of a triangle |
| LineLen | Line length |
| RotSD | The standard deviation (in degrees), is used to determine the |
| | rotation of the primitives when they are placed in the feature |
| CircArea | The area of circle primitives |
| TriArea | The area of triangle primitives |
| LineArea | The area of line primitives |
| DT | The number of triangle primitives scaled by feature area |
| DL | The number of line primitives scaled by feature area |
| CA | The total area of circle primitives scaled by feature area |
| TA | The total area of triangle primitives scaled by feature area |
| CCD | Number of colour changes of the feature over the diagonal |
| Pattern Count² | Number of micro-pillars per TopoUnit area |
| Area² | The actual number of pixels in the region |
| Compactness² | The variance of the radial distance of the object's pixels from the |
| | centroid divided by the area |
| Eccentricity² | The eccentricity of the ellipse that has the same second-moments |
| | as the region. The eccentricity is the ratio of the distance between |
| | the foci of the ellipse and its major axis length. The value is |
| | between 0 and 1. (0 and 1 are degenerate cases; an ellipse whose |
| | eccentricity is 0 is actually a circle, while an ellipse whose |
| | eccentricity is 1 is a line segment.) |
| Extent² | The proportion of the pixels in the bounding box that are also in |
| | the region. Computed as the Area divided by the area of the |
| | bounding box. |
| Form Factor² | Calculated as 4*π*Area/Perimeter2. Equals 1 for a perfectly |
| | circular object. |
| Major axis length² | The length (in pixels) of the major axis of the ellipse that has the |
| | same normalized second central moments as the region. |
| Min Feret | The Feret diameter is the distance between two parallel lines |
| Diameter² | tangent on either side of the object (imagine taking a caliper and |
| | measuring the object at various angles). The minimum Feret |
| | diameter is the smallest possible diameter, rotating the calipers |
| | along all possible angles. |
| Median Radius² | The median distance of any pixel in the object to the closest pixel |
| | outside of the object. |
| Max Radius² | The maximum distance of any pixel in the object to the closest |
| | pixel outside of the object. For skinny objects, this is 1/2 of the |
| | maximum width of the object. |
| Orientation² | The angle (in degrees ranging from −90 to 90 degrees) between |
| | the x-axis and the major axis of the ellipse that has the same |
| | second-moments as the region. |
| Perimeter² | The total number of pixels around the boundary of each region in |
| | the image. |
| Solidity² | The proportion of the pixels in the convex hull that are also in |
| | the object, i.e. ObjectArea/ConvexHullArea. Equals 1 for a solid |
| | object (i.e., one with no holes or has a concave boundary), or <1 |
| | for an object with holes or possessing a convex/irregular |
| | boundary. |
| InscribedCircle | The number of inscribed circles of a defined minimum diameter |
| number² | found between objects |
| Pillars Number | The total number of pillar primitives in the topo unit |

Each micro-topographical element contains primitives (circles, triangles and rectangles). Features are repeated to cover the surface of a TopoUnit.² For each of the descriptors derived from Image Analysis of brightfield images, area and shape features are extracted, each parameter has an additional subset of descriptors including; standard deviation, mean, median, mad, minimum, maximum, variance, skewness, mode and percentile (0.1, 0.25, 0.5, 0.75 and 0.9) measurements.

### Example 1 Identification of Microtopographies and Features of Microtopographies which Reproducibly and Predictably Modulate Bacterial Attachment

### SUMMARY

A high throughput method assessing bacterial attachment on 2,176 distinct combinatorial generated micro-topographies (TopoChip) was used to identify key surface parameters for bacterial attachment. A predictive model to identify key topographical patterns and their pro- and anti-attachment properties has been developed. Real time monitoring of the spatio-temporal surface colonisation provided insights into the resistance mechanism of the lead topographies which can have wide application where bacterial biofouling is a problem such as biomedical device centred infection.

Bacterial-TopoChip Attachment Screening

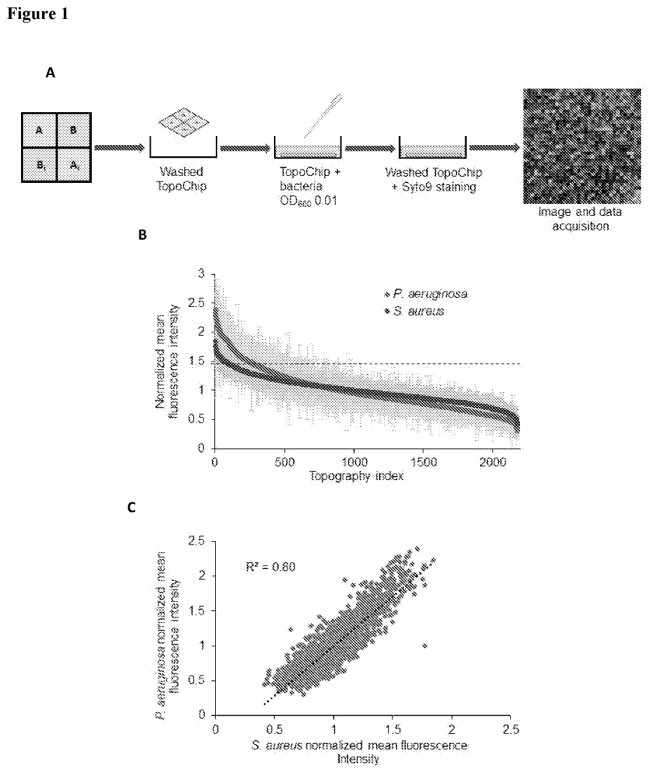

Firstly, the attachment of the human pathogen Pseudomonas aeruginosa was tested on PS TopoChips. To mimic in vivo conditions and stimulate bacterial attachment, the arrays were immersed in tryptic soy broth (TSB) medium supplemented with 10% human serum (TSBHS10%) at 37° C. for 4 h under static or flow conditions by incubating in an orbital shaker at 60 rpm, after which bacterial attachment was surveyed using fluorescent staining. The incubation time selected provided a sufficiently stringent assay for the identification of topographies both reducing and increasing initial bacterial attachment relative to the flat control (FIG. 1A). Although results showed a similar trend for both static and flow conditions, with P. aeruginosa attachment highly dependent on the local landscape, static incubation was selected for the screening as this condition produced consistent bacterial attachment while preventing the formation of biofilm streamers on TopoChip corners which can induce cross contamination of TUs under flow (Rusconi et al., 2010).

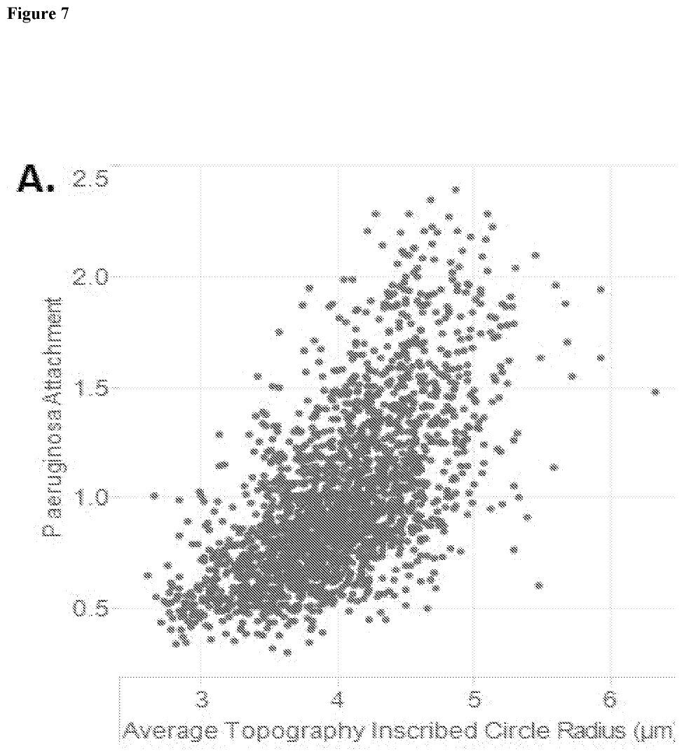

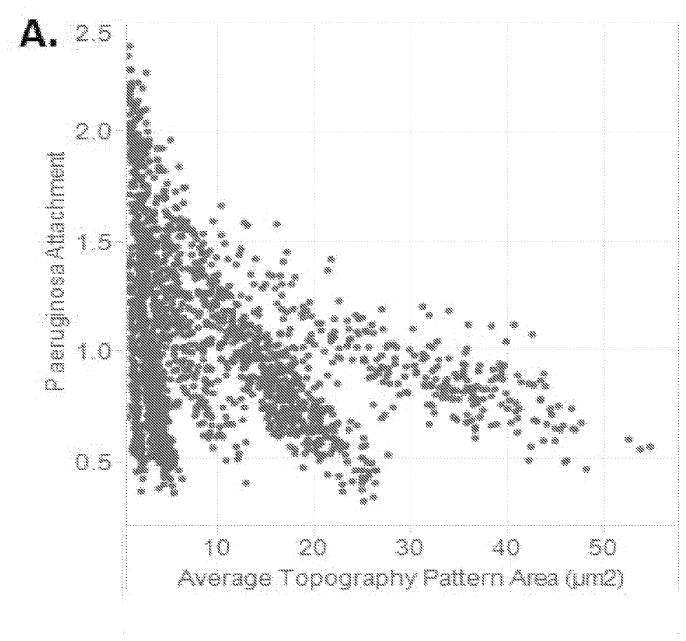

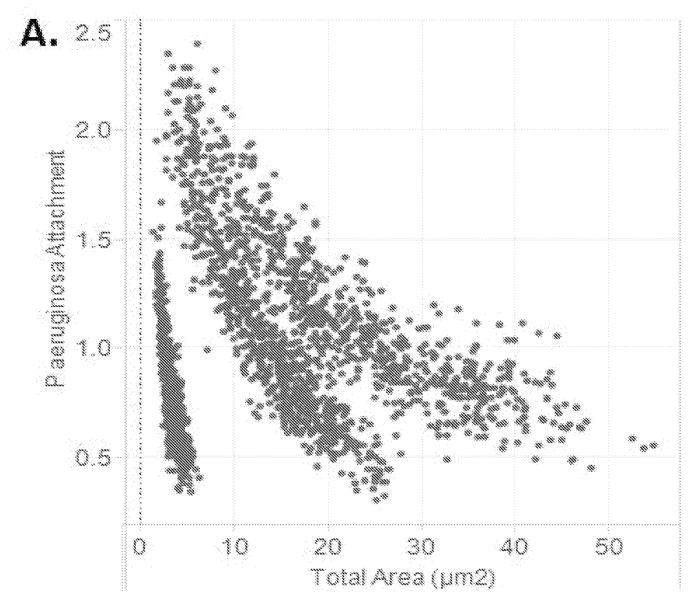

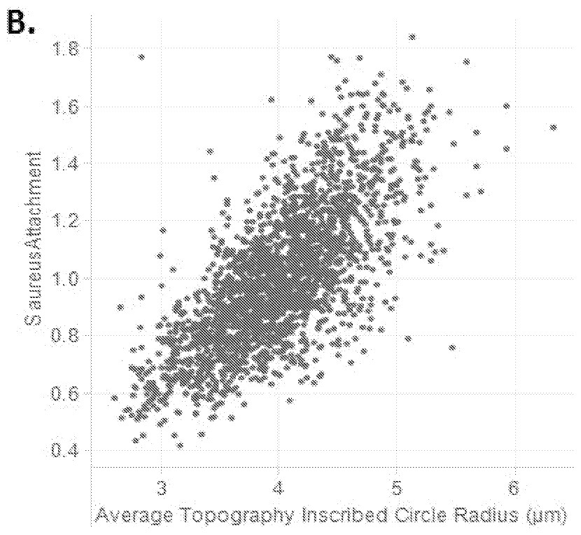

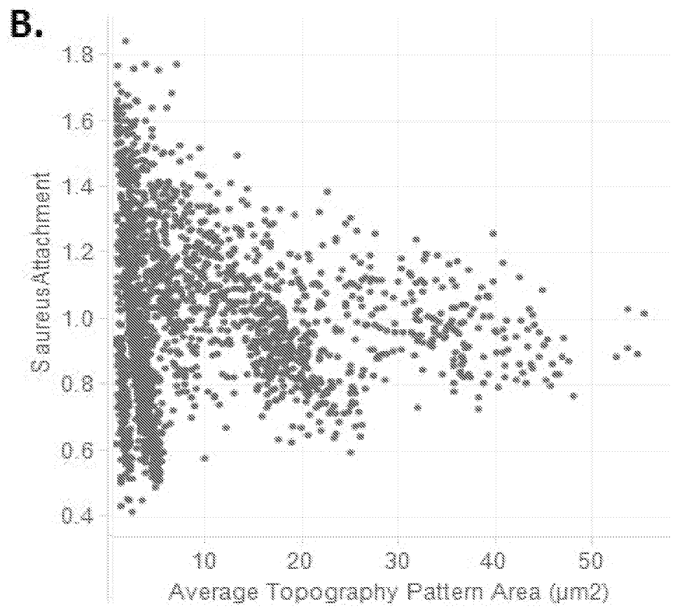

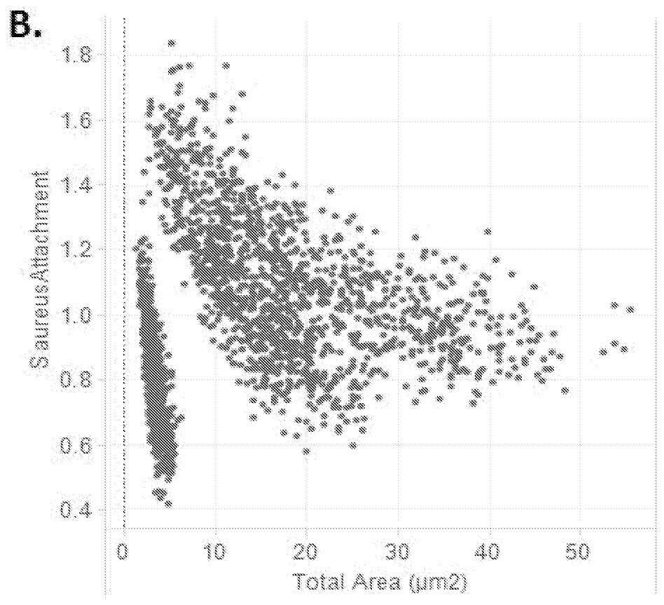

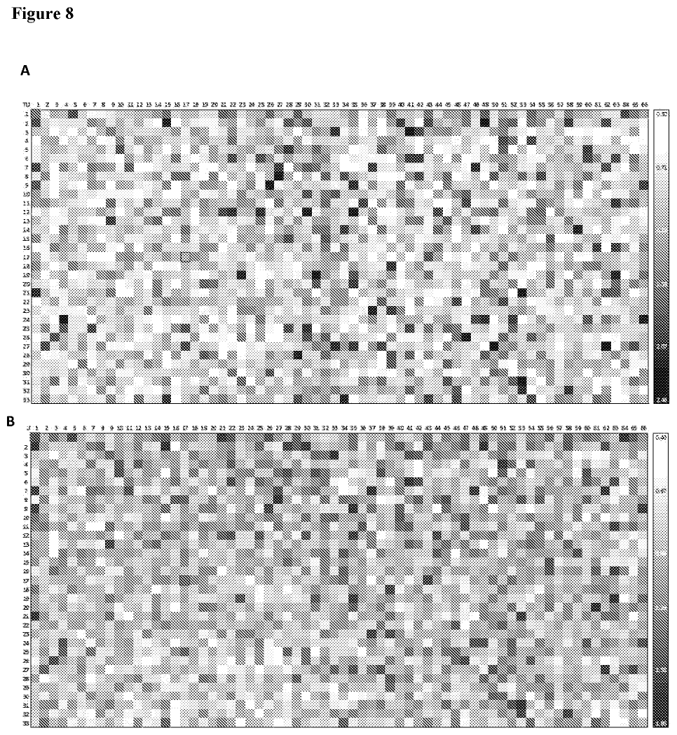

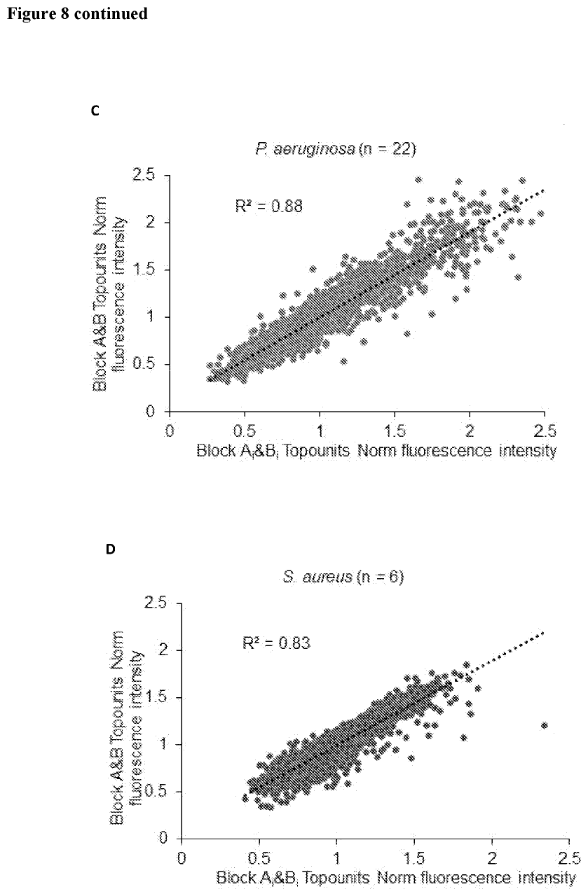

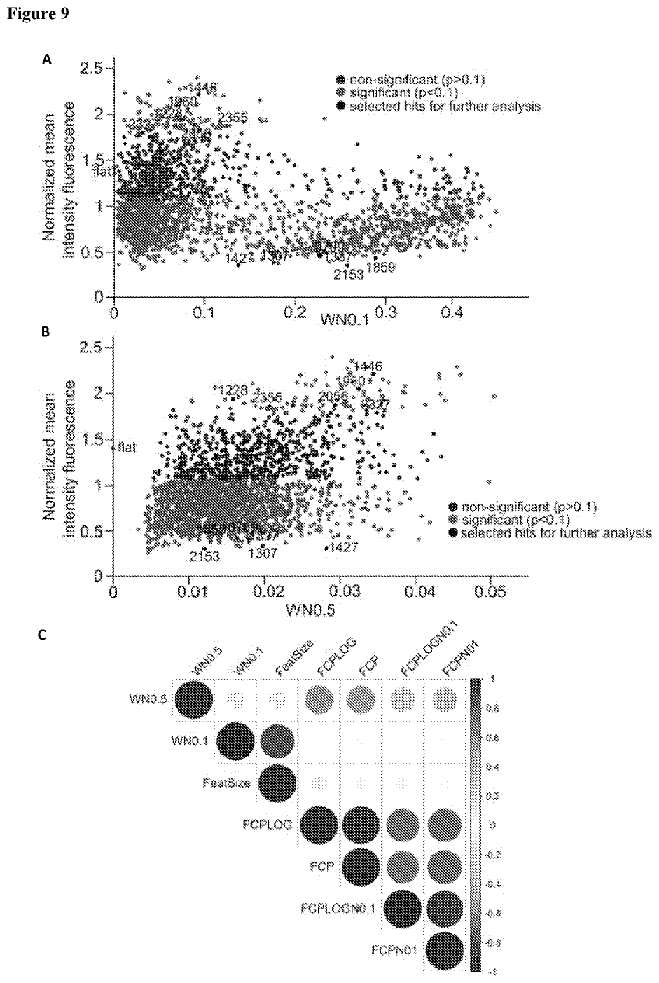

P. aeruginosa attachment to TUs in the PS TopoChip (n=22) revealed a wide range of micro-topographies showing resistance to bacterial attachment, with a strong association between P. aeruginosa attachment and specific types of micro-topographies (FIG. 1B and FIG. 7A). Although fluorescence intensity differences between pro- and anti-attachment topographies were less intense, a remarkably similar attachment pattern and TU-attachment correlation was observed for the Gram-positive pathogen S. aureus (n=6) after 4 h incubation in TSB at 37 C under static conditions (FIG. 1B, C, SFIG. 7B-D). Interestingly, most micro-patterns in the PS TopoChip had decreased bacterial attachment respect to flat control, especially for S. aureus with only 3% of the topographies increasing cell attachment (FIG. 1B). This is a surprising result since the presence of narrow grooves and cavities promotes bacterial retention on surfaces under static conditions (Whitehead & Verran, 2006; Shi & Zhu, 2009). These findings suggested that by modifying surface structure with well-defined micro-topographies one can control early cell attachment and subsequently reduce bacterial biofilm formation. Furthermore, and consistent with previous studies testing P. aeruginosa and S. aureus attachment on topographically defined surfaces under static and flow conditions (Graham & Cady, 2014; Chung et al., 2007), micro scale pillar size and channel width appeared to instruct bacterial cell attachment to certain topographies in the array. By visual inspection of the high and low-adhering topographies it appeared that less bacterial cells attach to topographies with smaller features displaying narrower spacing. To investigate this observation across the whole library of TUs in an unbiased manner, computational tools were applied to identify key surface parameters for bacterial attachment to the library of micro-patterns.

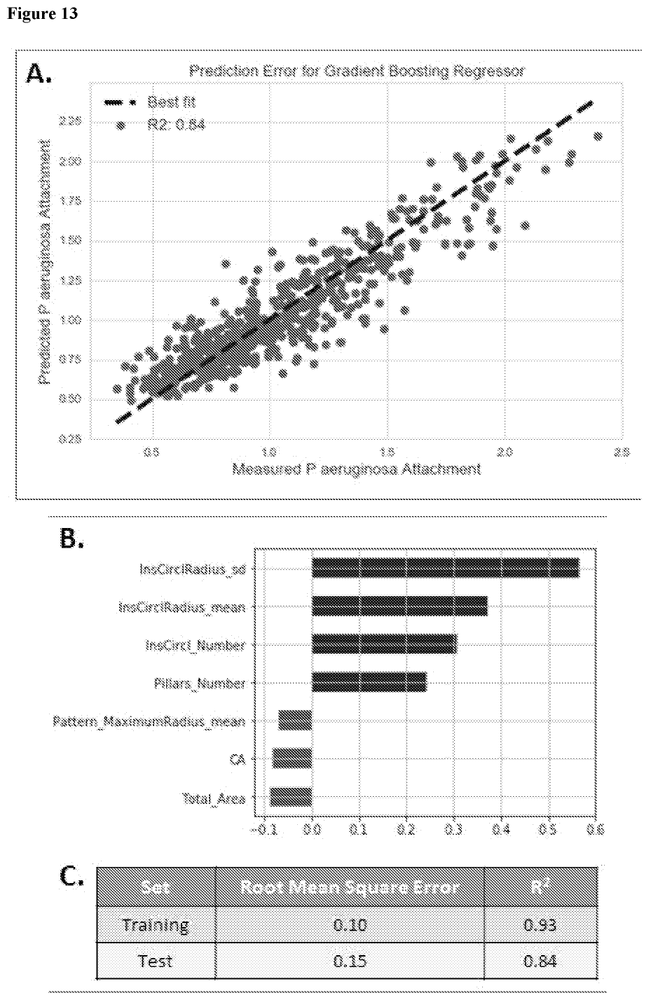

The performance of the P aeruginosa attachment model is shown, FIGS. 13A-D and results for the S aureus are shown FIGS. 13E-H. The contributions of the descriptors found to be the most important for each model are shown in FIG. 13B for the P aeruginosa dataset and in FIG. 13 F for the S aureus dataset. How each descriptor has contributed to the model is represented by colour bars in the figures (blue=increased bacterial attachment, red=reduced).

There were strong correlations between topographical descriptor based predictions versus observed attachment values for both bacterial models, with R²=0.84 and root mean square error (RMSE) 0.15 log fluorescence for P aeruginosa and R²=0.80 and RMSE 0.10 log fluorescence for S aureus in the test set using the ten most significant descriptors identified by the descriptor selection machine learning method. The spaces between objects (inscribed circle radii) and the area of the topographical features (both the total area and the area of the pillars) were some of the most important factors for predicting bacterial attachment. Specifically, features with a mean area<50 μm² and inscribed circle radii of >4 μm were associated with the highest P aeruginosa attachment (FIG. 7A). For S aureus, high attachment was associated with pillars with pattern areas of <10 μm², with a maximum inscribed circle of >4.5 μm (FIG. 7B). We plotted the bacterial fluorescence intensity against the dominant topography descriptor identified (standard deviation of the inscribed circles radii) to ascertain its predictive power alone and found that a linear function with an R²=0.49 and RMSE 0.25 is obtained for P. aeruginosa and R²=0.63 and RMSE 0.14 is obtained for S. aureus, indicating its dominance in predicting bacterial colonisation (FIGS. 13 D and 13H). The less dominant descriptors (average inscribed circle radii, total area and average topography pattern area) provide multiple solutions influenced by the values chosen for the feature dimensions (FIG. 7).

For biomedical purposes, topographies associated with low pathogen attachment and biofilm formation are interesting. Results from the regression model coefficients inform the magnitude of the topographical feature descriptors contributing to the attachment. Topographical descriptors with large negative coefficients are associated with low pathogen attachments (FIG. 13B and FIG. 13F). For P. aeruginosa, the total area of circle primitives scaled by feature area (CA), the total area occupied by primitives and the mean maximum radius of the pattern appear to influence low attachment (FIG. 13 B). For S. aureus (FIG. 13 F) the results indicate that the maximum area of patterns are negatively correlated to attachment. Examples of topounits at the extremes of the standard deviation of the inscribed circles radii and attachment are shown, illustrating that by eye these show the same characteristics as those observed previously.

It is remarkable that in these in vitro experiments with a gram-positive non-motile organism (S. aureus) and a motile gram-negative organism (P. aeruginosa) similar surface descriptors were found to dominate biofilm formation on polystyrene surfaces. To investigate the mechanism of sensing, a selection of the mutants were chosen.

Relevant Micro-Topographical Parameters Controlling Bacterial Attachment

CellProfiler analysis of images of the topography design provided 242 topographical shape descriptors. (Unadkat et al 2011.) Descriptors with Pearson correlation above 0.85 were eliminated, resulting in 66 which were used to train P aeruginosa and S aureus attachment models. The full set of topographical descriptors used for modelling is listed in Supplementary Table 1. The Extreme Gradient Boosting (XGBoost) machine learning regression method was used to identify correlations between the topographies and bacterial attachment, producing good models for the datasets. A descriptor selection machine learning approach was used prior to regression to eliminate less informative descriptors. Seventy percent of each dataset was used to train the models, and 30% were kept aside in a test set used to determine the predictive power of the models.

Surface Chemical Characterization of the TopoChip

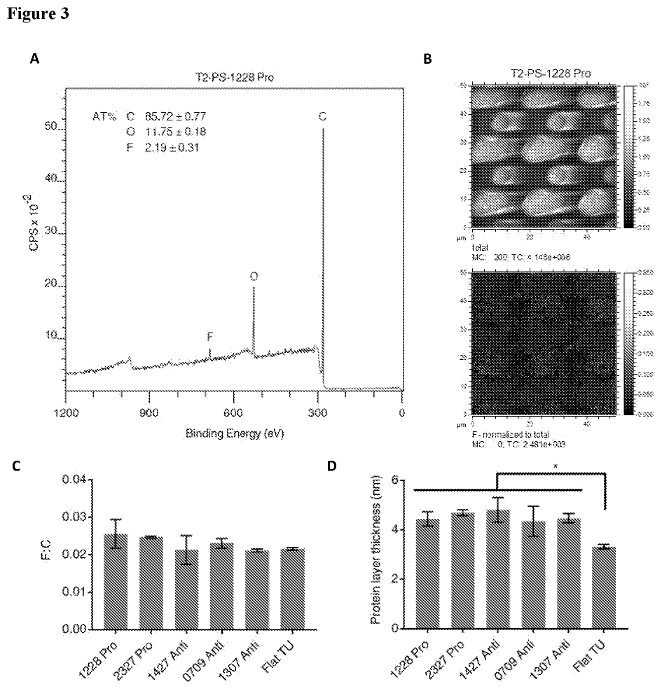

The chemistry of the PS TopoChip was analysed using techniques that probed the outermost surface of the array. These included time-of-flight secondary ion mass spectrometry (ToF-SIMS) for molecular characterization with high lateral resolution and X-ray photoelectron spectroscopy (XPS) for quantitative elemental analysis. Both methods detected fluorine impurities on the array surface, with XPS quantifying it for each TU on the array, e.g. topography T2-PS-1228 [F]=2.2±0.3 at % (FIG. 3A). This is assigned to residues from a monolayer of trichloro(1H,1H,2H,2H-perfluorooctyl)silane (FOTS) deposited on the OrmoStamp mould before using it in hot embossing of PS to facilitate the moulding procedure (Zhao et al., 2017). The distribution of fluorine present on the features, side walls and valleys was found constant using ToF-SIMS, within the limits of the technique imposed by the artefactual distortion of the features seen in FIG. 3B. Presenting a range of TUs where the fluorine to carbon ratio was quantified by XPS, FIG. 3C illustrates that there is no statistically significant difference between the units (one-way ANOVA, p=0.1398). These results indicate that the surfaces used in the screening have uniform chemistry and that the bacteria-material interaction observed is dependent on surface topography.

To investigate protein adsorption to individual TUs XPS analysis was carried out after incubation in TSBHS10% medium for 4 hours without bacterial cells. No significant differences in the estimate of the protein layer thickness were recorded between pro- and anti-attachment TUs (FIG. 3D). This further supports the hypothesis that differences in bacterial attachment are primarily based on the physical pattern structure. It also suggests that there are no large differences in total protein adsorbed to the surfaces influencing the bacterial response. Higher levels of nitrogen were detected on topographically defined surfaces compared to smooth control after medium conditioning, corresponding with an increase in estimated protein layer thickness (FIG. 3D), however it is thought that this difference originates from the reduced sampling depth on the vertical feature sides that overestimates the protein layer thickness.

Attachment Resistant Topographies and Biological Performance Assessment

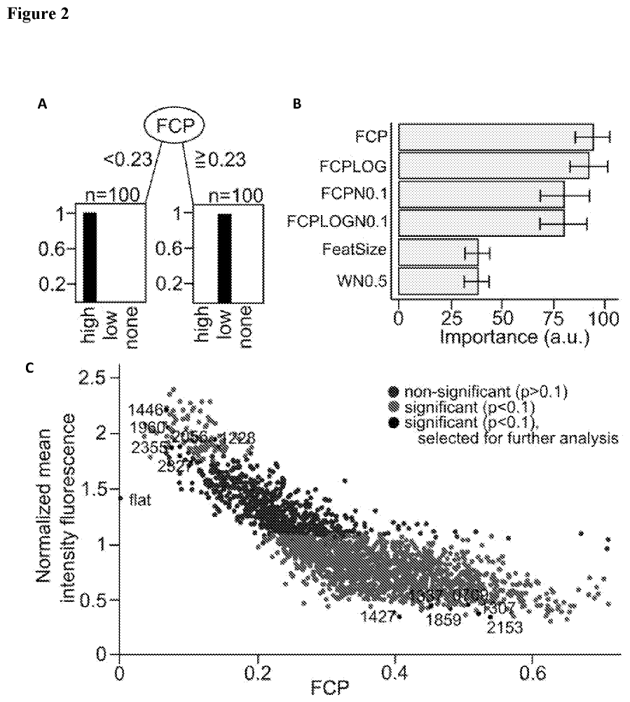

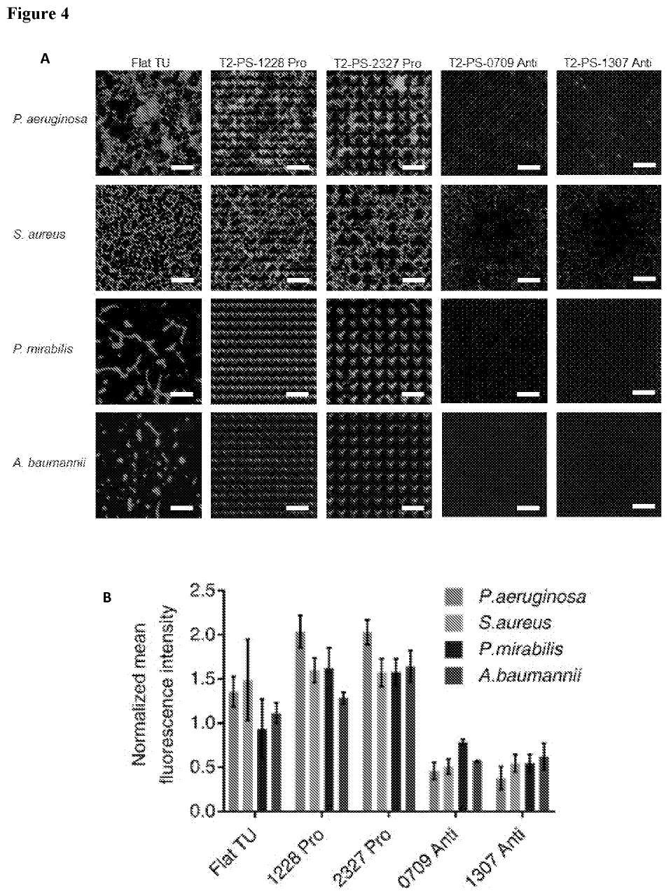

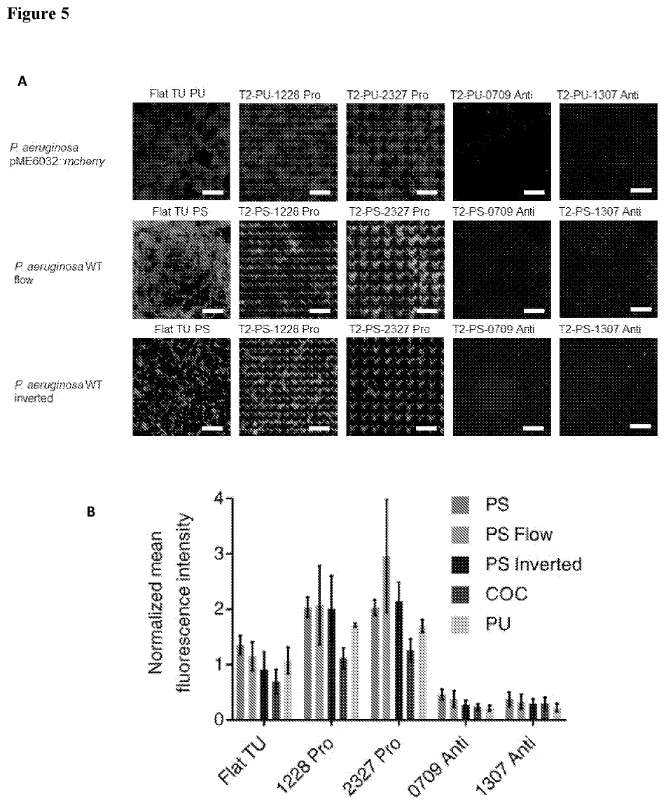

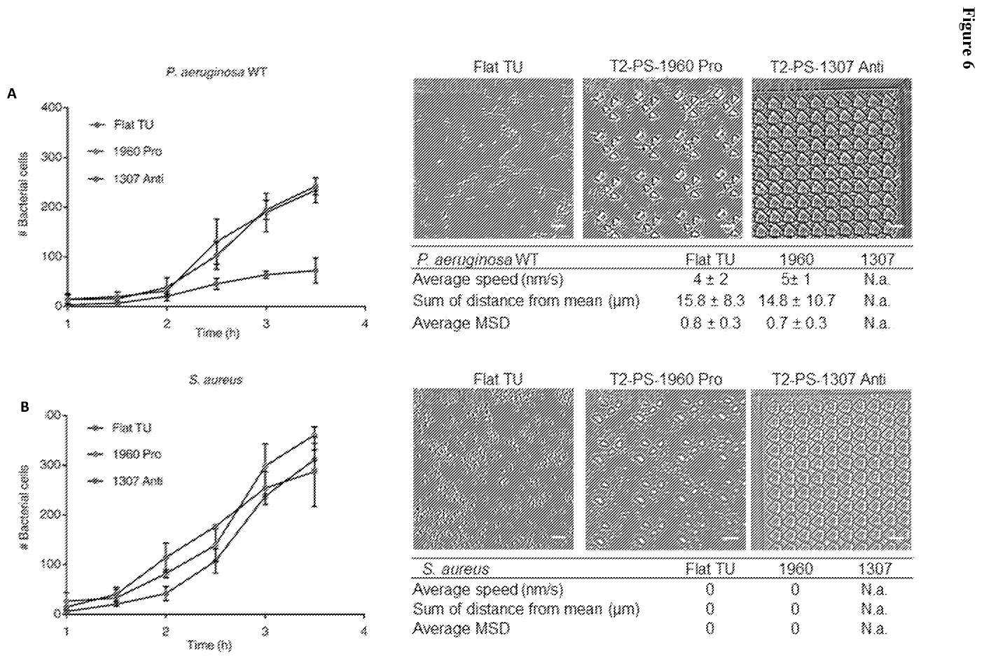

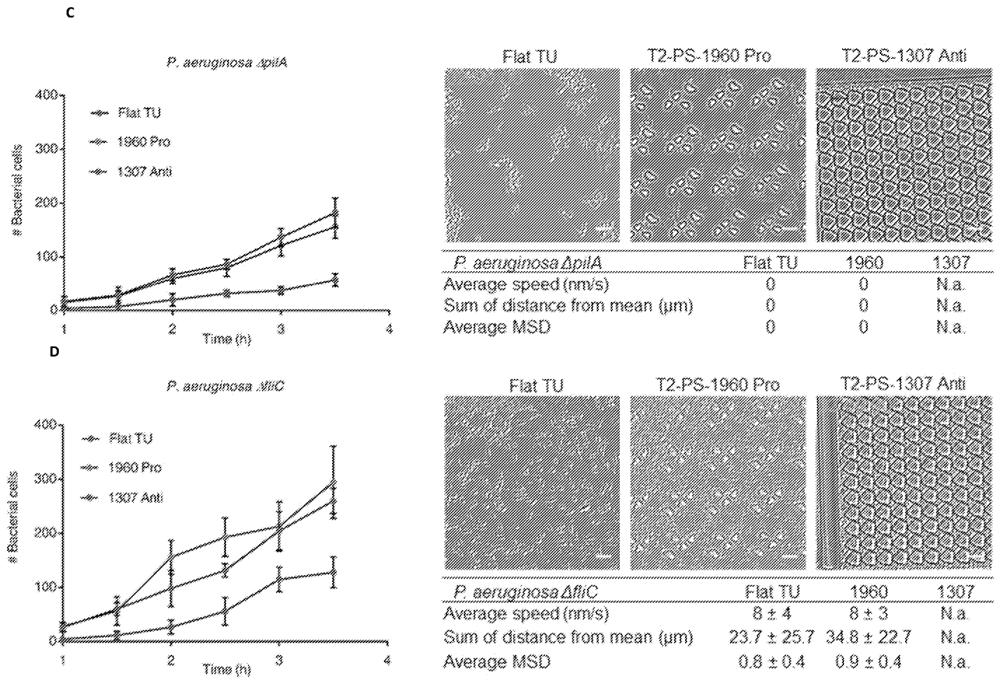

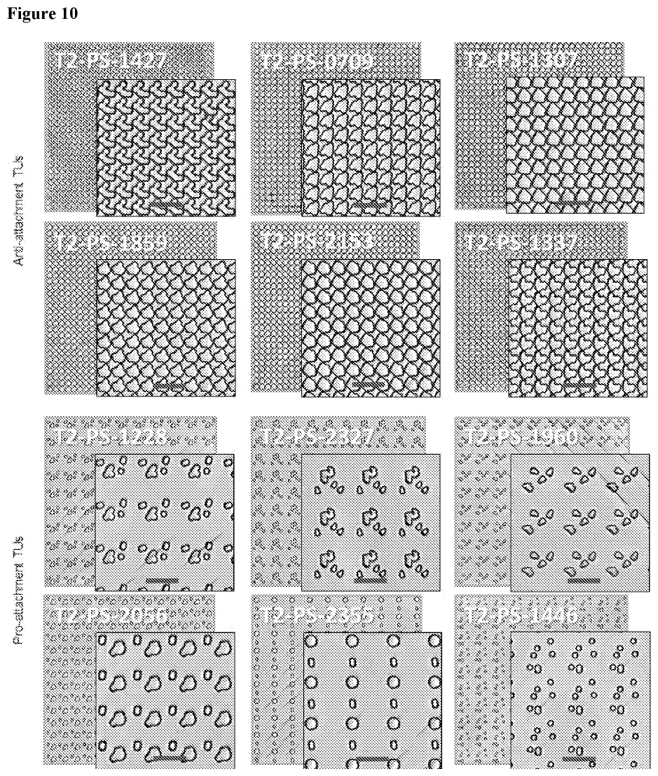

Based on the screening data obtained from quantification of P. aeruginosa and S. aureus attachment on PS TopoChips, hit topographies with anti-attachment (more than 2.6-fold decrease compared to smooth control surface) and pro-attachment (more than 1.25-fold increase) properties were selected for further studies (FIG. 2C, and FIG. 10). Interestingly, the selected TUs showed a similar biological performance when tested with other bacterial species including Proteus mirabilis and Acinetobacter baumannii grown in TSB at 37 C in static conditions for 12 h and 18 h respectively (FIGS. 4A and B). In order to further challenge selected TUs, P. aeruginosa attachment to hit micro-topographies was assessed under flow settings and with arrays incubated upside down oriented in the bacterial culture. These conditions were used to impede bacterial cells settlement due to gravitational effects and would require that bacterial cells move towards the topographies and actively seek suitable niches for attachment in the micro-patterns. Results showed that hit topographies maintained their pro- and anti-attachment properties against P. aeruginosa independently of the TopoChip orientation in the culture (FIGS. 5A and B).

To confirm that the differences in bacterial attachment to hit TUs are derived from feature arrangement, the biological performance of micro-topographies with enhanced and reduced attachment against P. aeruginosa was studied in TopoChips fabricated in the clinically relevant polymers PU and COC. Remarkably, the selected micro-topographies fabricated in both materials showed similar attachment levels to those in PS arrays providing further evidence that topography strongly influences bacterial attachment on these very different surface chemistries (FIGS. 5A and B).

Single-Cell Tracking and Surface Colonisation Analysis

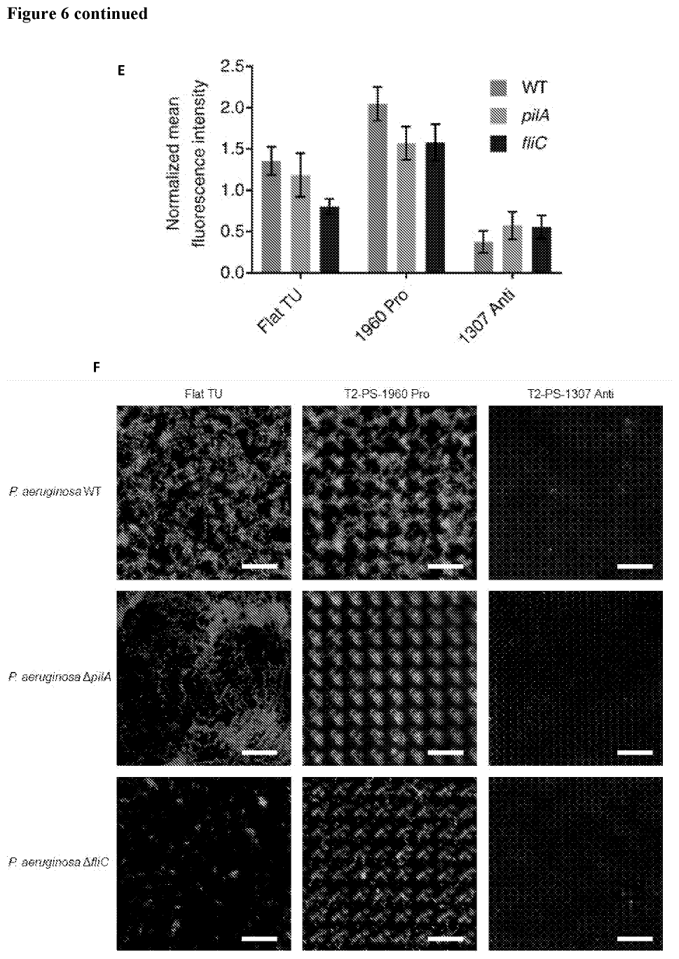

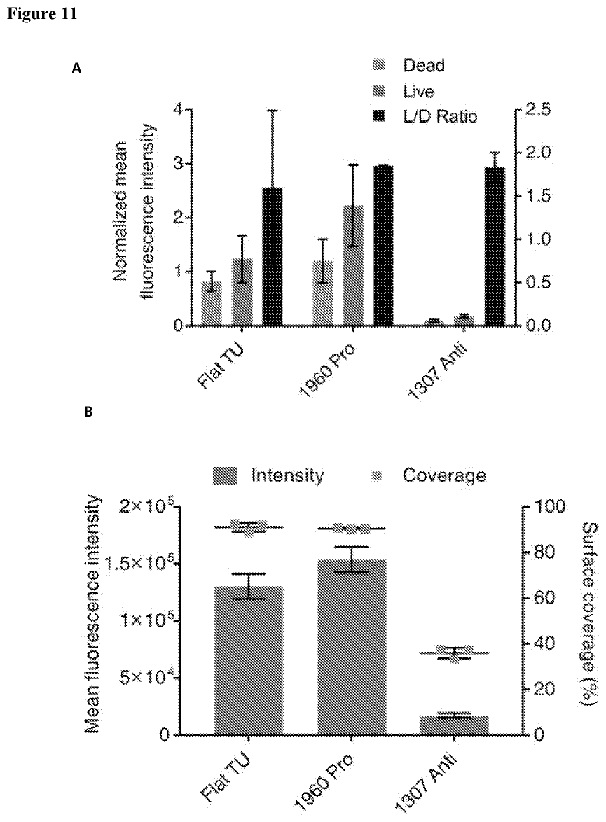

Since the lack of bacterial cell attachment observed on the anti-TUs could be a consequence of either surface avoidance or detachment following initial attachment, the behaviour of P. aeruginosa cells was monitored on flat and micro-patterned surfaces representative of increased and reduced attachment using time-lapse microscopy in static conditions. Live imaging and single cell tracking analysis showed a substantial reduction in the number of early (after 3.5 h) surface colonizing P. aeruginosa cells on an anti-attachment topography (T2-PS-1307) compared with a flat or a pro-attachment TU (T2-PS-1960) (FIG. 6A). Given the bactericidal effect described for various textured surfaces (Ivanova et al., 2012; Hasan el al., 2018; Wu et al., 2018) and to determine whether a similar mechanism was responsible for the reduced colonisation of anti-attachment TUs by P. aeruginosa, the viability of cells attached to the selected patterns was evaluated using live/dead differential cell staining and fluorescence microscopy. No significant changes in cell viability for the selected topographies was apparent (FIG. 11A) indicating that neither topography had a bactericidal effect on attached bacteria which could explain the differences in surface colonisation.

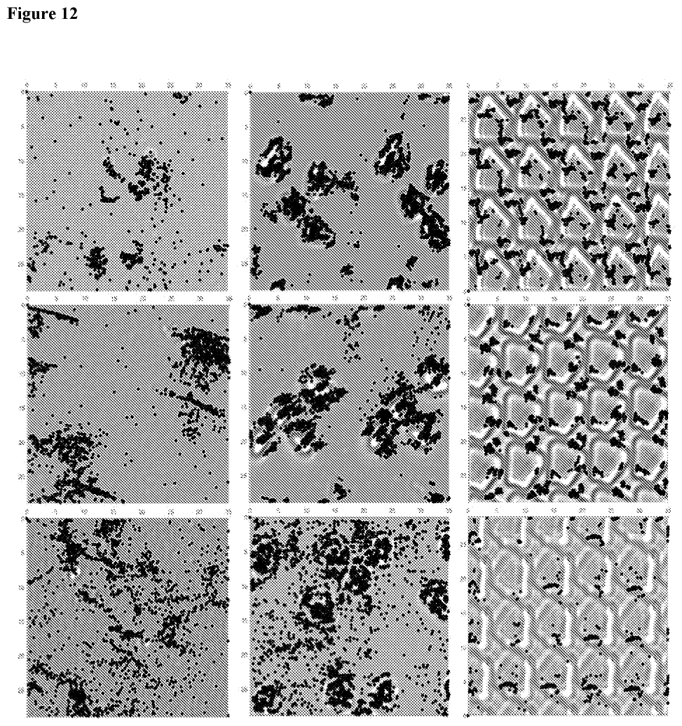

Real time imaging revealed that P. aeruginosa could enter all positions available in the tested micro-topographies, with cells moving freely in and out of the spaces between features and neighbouring niches within the Tus (FIG. 12). This suggested that the adhesion properties exhibited by hit TUs were not entirely related to prior bacterium avoidance/attraction to the surface but to attachment strength changes among different niches in the topographies.

To determine whether the non-motile Gram-positive S. aureus behaved similarly, the experiment was repeated under the same conditions. In contrast to P. aeruginosa, no significant differences in the number of staphylococci accumulating on the flat, pro- and anti-attachment TU surfaces colonisation was observed (FIG. 6b). However, in contrast to P. aeruginosa on the anti-attachment TU, staphylococcal cells were observed to settle on both the gaps between and tops of the micropillars.

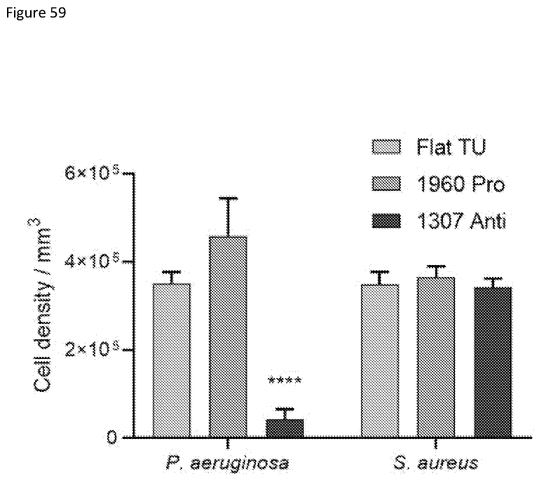

To investigate whether P. aeruginosa cells differentially accumulate similarly in the bulk medium immediately above the flat, pro- and anti-attachment TUs, 40 μm image stacks above the selected TU surfaces were captured after 3 h exposure and the cell population densities quantified. A significantly lower number of swimming P. aeruginosa cells was consistently recorded above the anti-attachment TU surface compared with the pro-attachment and flat surfaces (FIG. 59). In contrast, no differences in population cell density over the topographies were recorded for S. aureus over the same TUs (FIG. 59).

In order to study the influence of bacterial appendages on attachment to the selected TUs, colonisation and attachment of P. aeruginosa TFP (ΔpilA) and flagellum (ΔfliC) mutants were assessed. Single-cell tracking showed that surface-associated motility on all TUs was hindered in the ΔpilA mutant (FIG. 6C), indicating that this motility consists of mainly pili-mediated twitching in P. aeruginosa WT and ΔfliC strains (Conrad et al., 2011; Mattick, 2002). In contrast, the average speed and distance travelled by twitching cells in the ΔfliC mutant were enhanced compared to WT resulting in a faster colonisation phenotype (FIG. 6D), probably as consequence of flagellum deficiency which reduces the rate and strength of attachment to surfaces (Haiko & Westerlund-Wikstrom, 2013).

Live cell imaging also showed different colonisation phenotypes for the three strains of P. aeruginosa studied. Firstly, a slight increase in surface occupation of TUs by ΔfliC mutant compared to the parental strain and ΔpilA mutant was observed (FIG. 6D, compare with A and C), possibly due to a greater cell sedimentation owing to the lack of flagellum and gravitational effects. Notably, the higher surface exploration by ΔfliC strain in the anti-attachment TU did not produce an improvement in attachment levels to this topography compared to WT strain (FIG. 6E) suggesting that, as for S. aureus, depositing cells were not able to irreversibly attach to this surface. In agreement with previously published results (Conrad et al., 2011), cell aggregation was enhanced in the ΔpilA mutant compared to WT strain resulting in an uneven surface attachment phenotype on flat and pro-attachment surfaces (FIG. 6F). Moreover, higher number of microcolonies developed nearby the micro-pillars of the pro-attachment TU in ΔpilA (FIG. 6C), which corresponded with an increase in attachment around these niches and fewer cells attaching to spaces between features (FIG. 6F), indicating that only TFP-competent cells are capable of detaching and redistribute. This suggests that the micro-pillars in the pro-attachment TU may act as topographical extensions of the surface and maximize the contact area of bacterial cells with the substrate as proposed elsewhere (Whitehead & Verran, 2006). Altogether these results support a role of the flagellum in P. aeruginosa attachment to topographically defined surfaces, whereas pili could allow colonisation of micro-topographical surfaces producing uniform attachment on smooth and attachment-permissible niches.

CellProfiler analysis of topography images provided 66 uncorrelated topographical shape descriptors that were used to train P aeruginosa and S aureus attachment models (Unadkat et al, 2011) The full set of topographical descriptors is listed in Supplementary Table 1. For P. aeruginosa 2142 topo units were investigated and for S aureus 2172 were considered. Topo units were excluded from the analysis if their signal to noise ratio was lower than 2.

The XGBoost machine learning method and Multiple Linear Regression with Expectation Maximisation (MLREM) were both used to generate non-linear and linear relationships between the topographies and bacterial attachment, producing good models for the datasets. Those methods were coupled with Shappley Additive Explanation (SHAP) method (S. M. Lundberg, S. I. Lee, A unified approach to interpreting model predictions. Adv Neur In 2017, 30) for descriptor selection. The models were build based on the top ten most informative descriptors for each dataset, as identified by SHAP. All methods were implemented in Python 3.7. XGBoost version 0.22 using default parameters was employed to generate the ML models. (T. Q. Chen, C. Guestrin, XGBoost: A Scalable Tree Boosting System Kdd'16: Proceedings of the 22nd Acm Sigkdd International Conference on Knowledge Discovery and Data Mining 2016, 785). Seventy percent of each dataset was used to train the models, and 30% were kept aside in a test set used to determine the predictive power of the models.

Although XGBoost has produced a better non-linear fit to the data (with R²=0.84 and RMSE 0.15 log fluorescence for P aeruginosa; and R²=0.80 and RMSE 0.10 log fluorescence for S aureus in the test set), MLREM regression coefficients assisted informing the individual contribution of each descriptor to attachment (FIGS. 1B and 1F). MLREM results showed that there is also a strong linear correlation between the selected descriptors and bacterial attachment, with R²=0.77 and RMSE 0.19 log fluorescence for P aeruginosa; and R²=0.71 and RMSE 0.11 log fluorescence for S aureus in the test set.

TABLE 2
Important topographical descriptors identified for bacterial attachment

| Biological Outcome | Descriptors | % |
|---|---|---|
| High P aeruginosa | Inscribed Circle Radius Standard Deviation | 63 |
| Attachment | Pillar Number | 32 |
| Low P aeruginosa | CA | 10 |
| Attachment | Total Area | 36 |
| High S aureus | Inscribed Circle Radius Standard Deviation | 49 |
| Attachment | Inscribed Circle Radius Mean | 46 |
| | Inscribed Circle Number | 23 |
| Low S aureus | CA | 6 |
| Attachment | Pattern Area Max | 3 |

These descriptors are highlighted by mathematical and machine learning approaches to the dataset. Those approaches also allow the understanding of whether the descriptors impact the biological activity in a positive or negative manner.

Topographical descriptors are a set of structural properties and characteristics that describe the topographical surface of the materials. For instance, if there is a material with round pillars in the chip, examples of descriptors would be: number of pillars, size of individual pillar, space between the pillars etc.

In the above table the % indicates the percentage (variance) of the whole dataset that can be explained by a particular descriptor.

In Vivo Assessment of Pro and Anti-Attachment Topographies in a Murine Foreign Body Infection Model

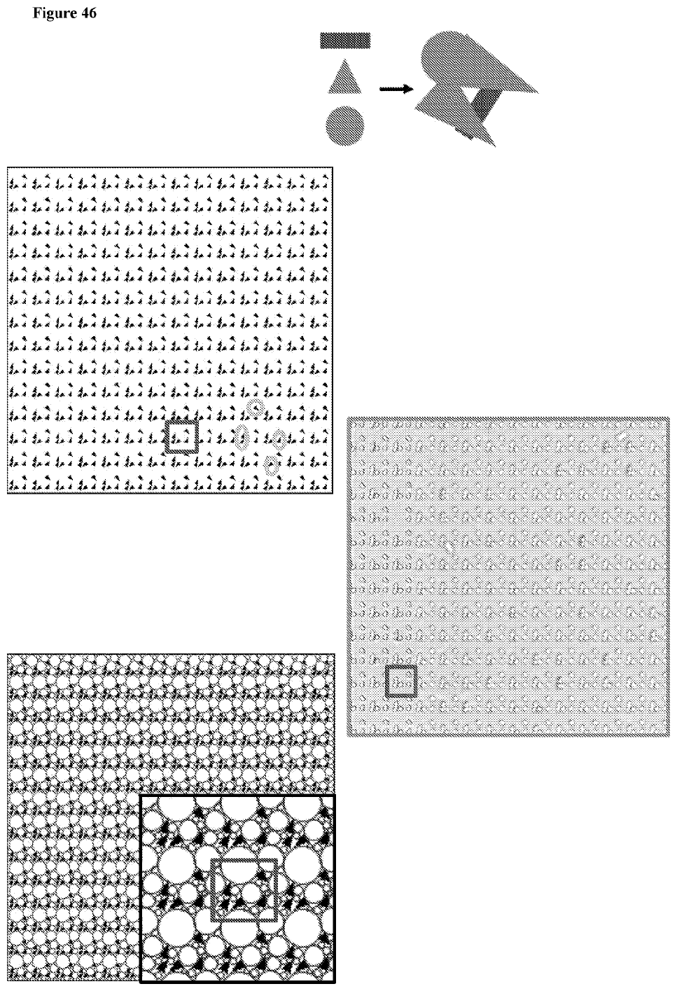

To determine whether surface conditioning by serum proteins influences the interactions of bacterial cells with pro- and anti-attachment TUs as well as to mimic in vivo conditions, we grew P. aeruginosa in TSBHS10% or TSB and compared attachment to flat, pro- and anti-attachment TUs after incubation at 37° C. for 4 h. FIG. 3D shows that serum protein layers of similar thickness as quantified using XPS were deposited on the pro- and anti-attachment TUs. The comparative attachment behaviours of P. aeruginosa on the flat, pro- and anti-attachment TUs were not affected by serum protein deposition (new FIG. 46)

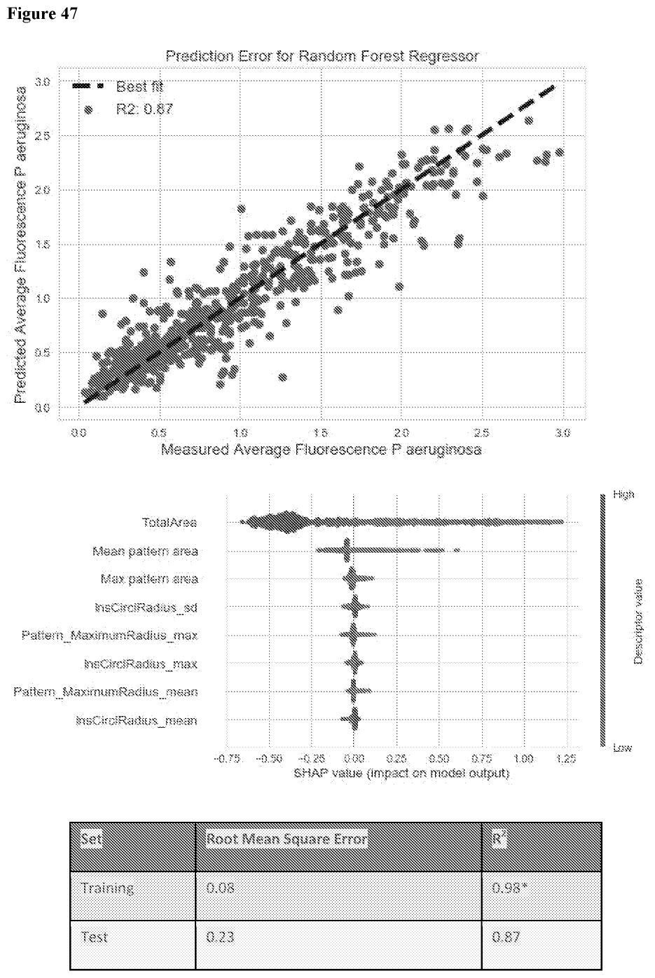

To explore the in vivo host response and bacterial attachment resistance, pro- and anti-attachment TUs were implanted subcutaneously into mice which, after recovery, were inoculated with either P. aeruginosa or PBS (uninfected control). After 4 days, the TUs were removed and their micro-topographical integrity confirmed by scanning electron microscopy (FIG. 47). The semi-quantitative bacterial attachment and host response data are summarized in Table 3 and illustrated by FIG. 48. No bacterial cells were detected on the uninfected control TUs. These data contrast with the infected pro-attachment TUs (T2-PU1228 and T2-PU2056) where P. aeruginosa cells were clearly detectable and a robust host response observed. Much lower levels of both P. aeruginosa and host cells were apparent on anti-attachment TUs such as T2-PU1307 after removal from the infected animals (Table 3 and FIG. 48).

TABLE 3
Host cellular response to pro- and anti-attachment
Topo-units in mice infected with P. aeruginosa.

| | | P. | | | |
|---|---|---|---|---|---|
| | | aeruginosa | Fm1-43 | CD206 | CD45 |
| | | Bacterial | Total Cell | Macro- | Leuco- |
| | Feature ID | Detection | Biomass | phages | cytes |
| Pro- | T2-PU-1228 | ++ | +++ | ++ | +++ |
| attachment | T2-PU-2056 | +++ | +++ | ++ | +++ |
| Anti- | T2-PU-0709 | − | + | + | +++ |
| attachment | T2-PU-1307 | − | + | + | +++ |
| | T2-PU-1429 | + | ++ | + | +++ |
| | T2-PU-2153 | + | ++ | + | +++ |
| Control— | T2-PU-1307 | − | + | +/− | + |
| no | T2-PU-1429 | − | + | +/− | + |
| bacteria | T2-PU-2153 | − | + | +/− | + |

−, not detected;
+, low level;
++, intermediate;
+++, high level;
see FIG. 48 for examples of the corresponding confocal fluorescence microscopy images. Data from 3 TUs per microtopography combined.

### DISCUSSION

The interplay between bacterial cells and topographical landscapes is still poorly understood. Here, the previously described micro-topographical array “TopoChip” (Unadkat et al., 2011) was exploited to discover surfaces that prevent bacterial attachment and gain insights into the interface between specific topographical landscapes and bacterial cells.

The high number of unique micro-topographies assessed (2,176) and the remarkably strong correlation found between local landscape and bacterial attachment, allows a detailed analysis on the relevance of underlying surface design parameters on biological responses and exploring an innovative approach for antifouling surface engineering which predicts bacterial attachment based on surface design criteria rather than single surface traits such as surface energy or water contact angle. The results reveal that the surface parameter FCP influences attachment of both P. aeruginosa and S. aureus to the tested micro-topographies in a reproducible and predictable mode. This predictor provides information on the size and density of the micro-pillars into bounding squares, referred to as features in this description. Generally, the higher the FCP value of the micro-topography the less bacterial cells attach to it. This trend is modulated, however, by the feature size (Featsize) and the intricacy of the protruding elements in the pattern as indicated by the relevance of the Fourier transformation of the patterns (Wavenumber—WN), a mathematical representation of the spatial arrangement of the topographical elements on the surface. Therefore, high FCP micro-topographies comprising low scoring WN further arrest bacterial attachment, whereas features including many details or constituted by several micro-pillars (high WN) would favour attachment even in high FCP scoring topographies, a finding which could not be anticipated from the current understanding of bacteria-surface interactions. These outcomes illustrate the strength of unbiased screenings of large topographical libraries to reveal previously unperceived cell-surface interactions as well as provide insights towards the rational fabrication of new bioactive surfaces. An alternative strategy for antibacterial surfaces development is the nature-inspired approach that has been successfully applied by several groups to generate topographies that mimic naturally occurring antifouling surfaces (Cheng et al., 2006; May et al., 2014; Li et al., 2016). The difficulty of this strategy, however, resides in the need of previous knowledge of potential bioactive surfaces and discover ways to improve their attachment resistance properties. The active learning approaches used in this work are especially suited for the latter since biological outcomes can be correlated to the surface design criteria and iterative testing would be required to optimize topographies.

Using different pathogens growing on the TopoChip platform, the inventors have revealed new bioactive micro-topographies and defined features that support or reduce bacterial attachment. Moreover, similar biological performance was recorded for hit micro-patterns fabricated in different polymer materials indicating that the changes observed in bacterial attachment depend on the topographical features rather than surface chemistry. Real time imaging of selected topographies exposed to growing bacterial cultures showed that cells could gain access to all niches available within selected topographies. This was expected since conventional wisdom states that the width and spacing of the topographical features in a pattern should be adapted to the size of the organism to prevent biofouling, yet the feature sizes encountered in the TopoChip are significantly bigger than bacterial cells (10, 20 or 28 μm). These observations suggest that the changes in attachment levels observed on tested surfaces are not related to bacterial cells being barred from attachment restrictive areas but to cells sensing and responding in different ways to the niches available. Therefore, in contrast to the use of coatings to combat bacterial fouling (i.e. incorporating biocides), the anti-attachment mechanism based on micro-topographical modification of surfaces may reduce bacterial attachment below clinically-relevant levels without posing selective evolutionary pressure on microorganisms to develop antimicrobial resistance. Hence, this approach could be used to texturize the surface of biomaterials commonly used in clinical settings with the benefit of retaining their physical and mechanical properties as well as reducing the cost of new materials discovery and testing.

The development of the topochips is described in FIG. 46. FIG. 46a shows the use of 3 primitive shapes—namely a rectangle, triangle and circle that are combined to form a micropillar structure. Typically the micropillars are shaped according to a topography determined using a screening technique of possible primitive shape combinations. The primitive shape may be combined using a computational algorithm to generate a hybrid shape or micropillar that does not resemble the original primitives. It can be appreciated that such a hybrid shape may be a single conjoined shape, or may be a collection of shapes, in which case the micropillar is considered to include all shapes in the collection. The micropillars are then arranged on the surface in a repeating patterned array. This is shown in FIG. 46b which is a mathematical image of micropillars (circled in yellow) that together form a feature (red) that is repeated across the surface.

The micropillar features together form a topounit as shown in FIG. 46c, that is a brightfield image of such a topochip. The feature is shown in the box (Red). In the example shown this is a 290×290 μm.

An alternative way of considering the effect of the features on the surface is to consider an inscribed circle analysis of the descriptors. This is shown in FIG. 46d, where circles are used to define space between the micropillars that form the feature and also to adjacent features. Accordingly, in addition to interaction between the shapes or morphology of a single micropillar, cellular processes may be influenced by adjacent micropillars

The micropillars are formed of surface structures between 1-100 μm in height, and 1-50 μm in width, wherein said microtopographic pattern acts to modulate one or more cellular processes on the surface. By varying one or more of Total area-area covered by features in the topounit; Mean pattern area-average area of the pillars per feature; Max pattern area-area of the biggest pillar per feature; Ins Circle radius sd-standard deviation of the radius of the inscribed circle; Ins Circle radius mean-average of the radius of the inscribed circle; Ins Circle radius max-maximum of the radius of the inscribed circle; Pattern maximum radius max-biggest radius of the biggest pillar in a feature; Pattern maximum radius mean-average radius of the biggest pillar in a feature; Total perimeter-total length of all the features in a given topounit.

The micropillar may be about 1-100 μm in height (vertical), such as about between 5-45 μm, 10-40 μm, 15-35 μm, 20-30 μm, 25 μm, or 50-100 μm in height. In one example the micro-pillar may be approximately 10 μm in height.

Similarly, the micropillars may be between 1-100 μm in width (lateral), such as 2-45 μm, 3-40 μm, 4-35 μm, 5-30 μm, 10-25 μm, 15-20 μm, or 50-100 μm. In one embodiment the micropillars are approximately 3 μm in width, such as 3.0+/−0.6 μm. Suitably, a micro-pillar may be 3-23 μm wide laterally and about 10 μm in height, such as 9.1+/−0.6 μm

In one embodiment the microtopography of the micropillars above the underlying surface may have a mean area below 50 μm². In other embodiments, the micropillars have an eccentricity of <1, and preferably less than 0.5, preferably between 0.01-0.49, more preferable between 0.1-0.4, most preferably between 0.2-0.3.

FIG. 47 shows non-linear regression for the bacteria P. aeruginosa. FIG. 47a shows results for Random Forest Modelling with Top 8 Important Features selected by SHAP for the Test Set. In particular, the graph of FIG. 47b shows the important features, ranked from most to least important. SHAP scale shows negative/positive influence on the outcome. For instance, High values of TotalArea (pink) are more likely to have negative effect on Attachment. Mid range values for TotalArea can have both positive or negative (purple values); low values (blue) have positive effect on Attachment. The data is shown in FIG. 48, with the effect of each variable on the average fluorescence (i.e. attachment).

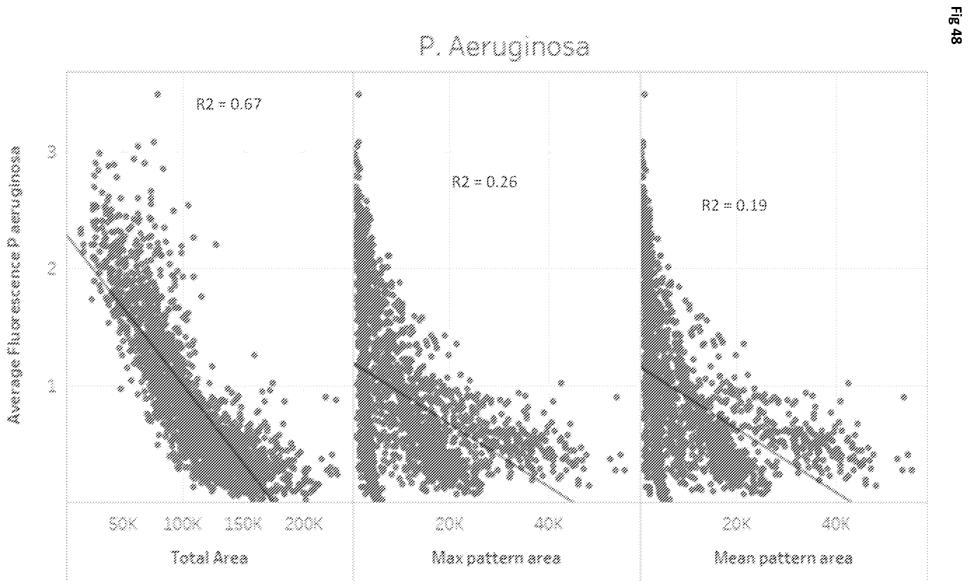

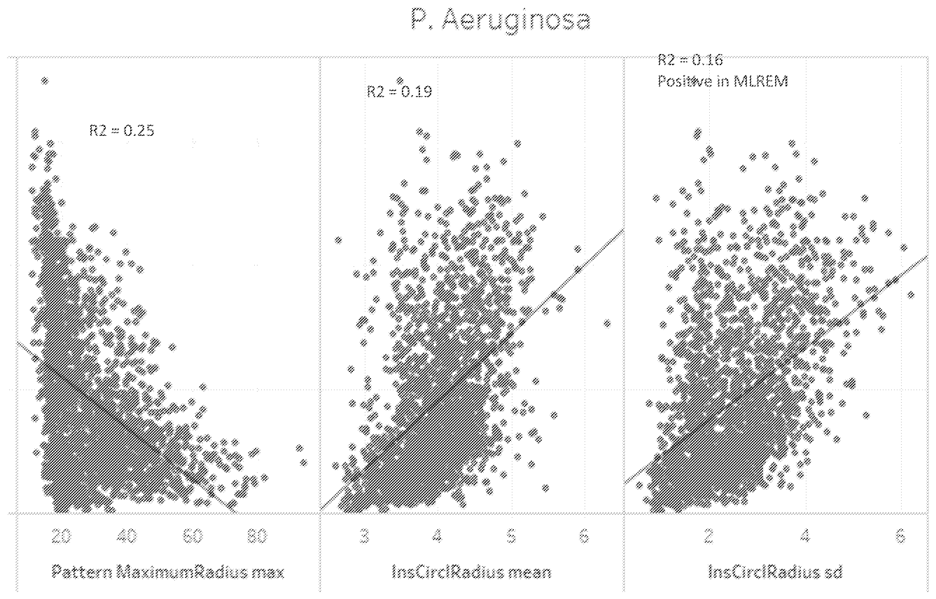

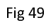

As shown in FIG. 49, a model with Pattern Area Max, InscrCirclesMean and InscrCirclesSD produces R2=0.71 in the model. So we can build good models without total area. Pattern Area Max is the area of the bigger pillar in a feature. FIG. 49 shows the graph of FIG. 48 converted to microns.

FIG. 50 shows the effect of attachment of the bacteria when analysed to topochips using the inscribed circles technique to define coverage. Strong anti-attachment topography cannot have radius mean≥3.2 px (0.32 μm) and SD≥1.4 px (0.14 μm).

FIG. 51 shows the attachment compared to the max patterned area. Where the max patterned area is the area of the biggest pillar in the feature. Here Medium/low-attachment≥170 μm2; High Attachment<40 μm2, but other factors need to be considered, such as the space between the pillars.

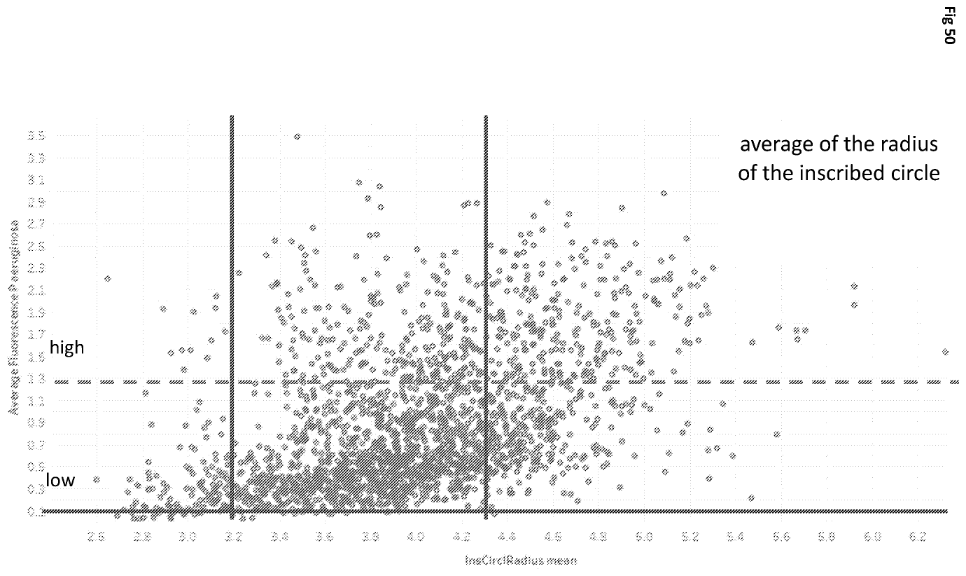

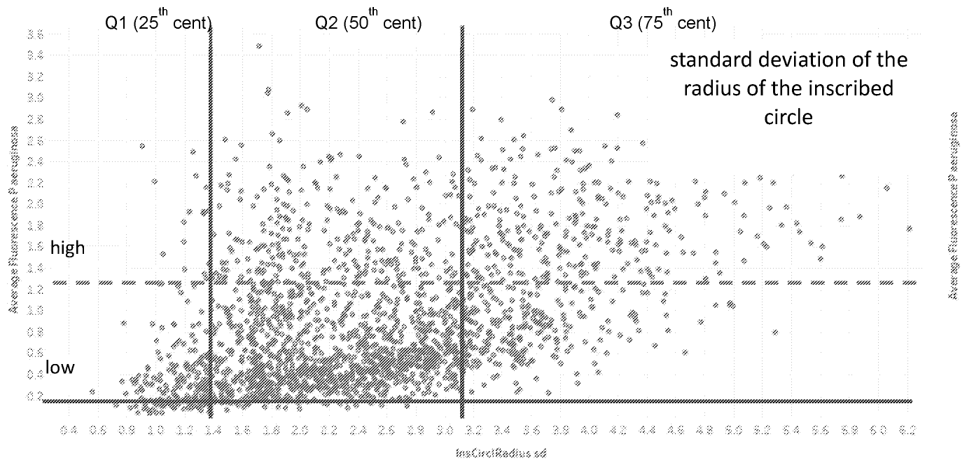

FIG. 52 shows the results for the bacteria S aureus. As shown in FIG. 52, Results for Random Forest with Top 8 Important Features selected by SHAP for the Test Set. The graph shows the important features, ranked from most to least important.

SHAP scale shows negative/positive influence on the outcome. For instance, High values of TotalArea (pink) are more likely to have negative effect on Attachment. Some mid range area topographies also affect negatively attachment. Mid range values for TotalArea can have both positive or negative (purple values); low values (blue) have positive effect on Attachment.

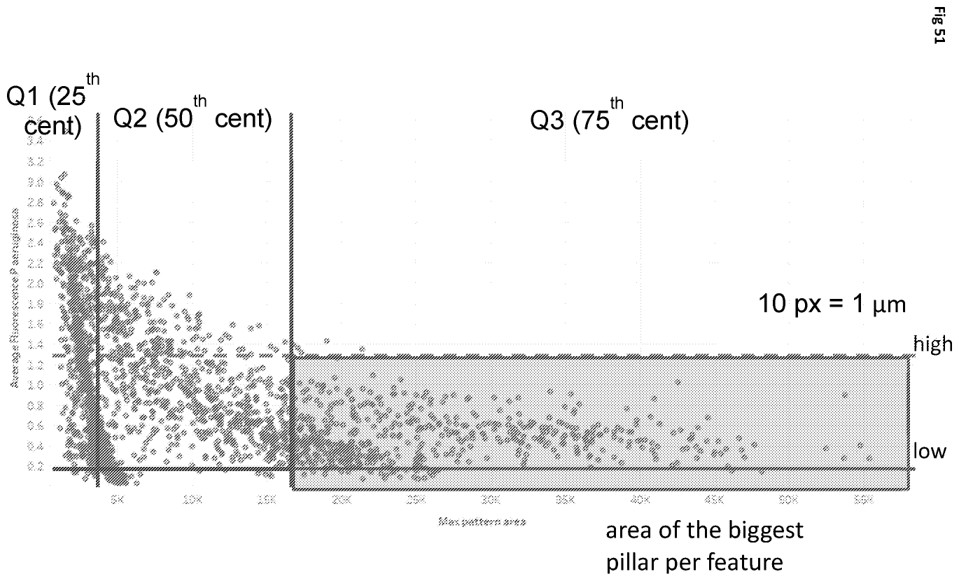

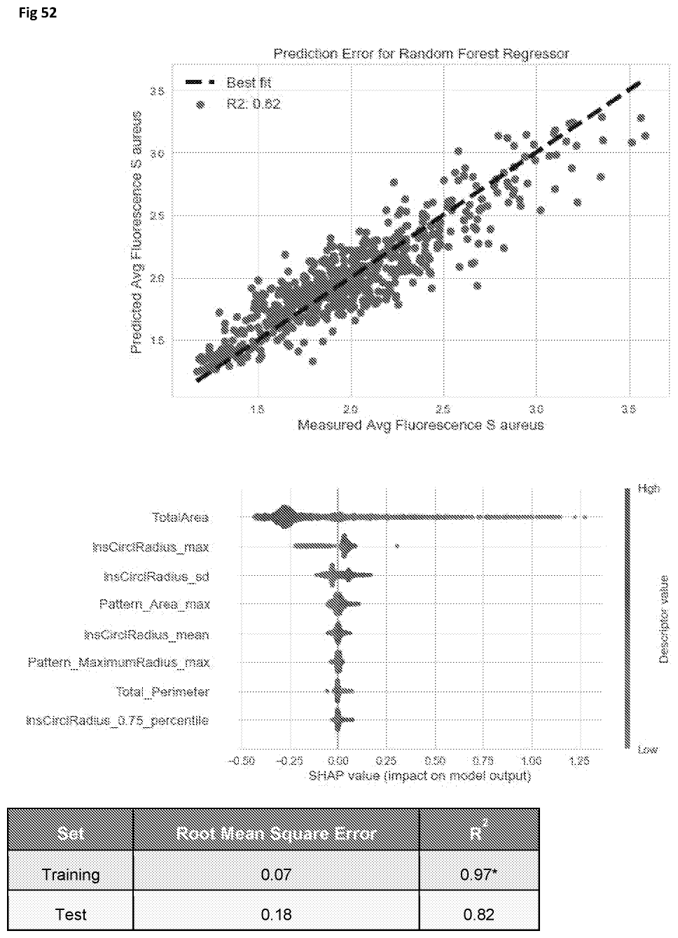

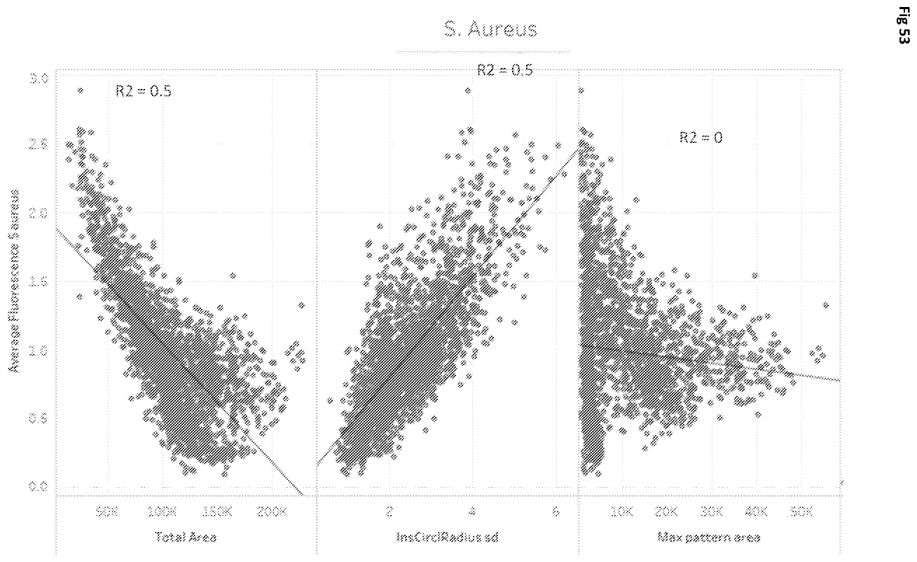

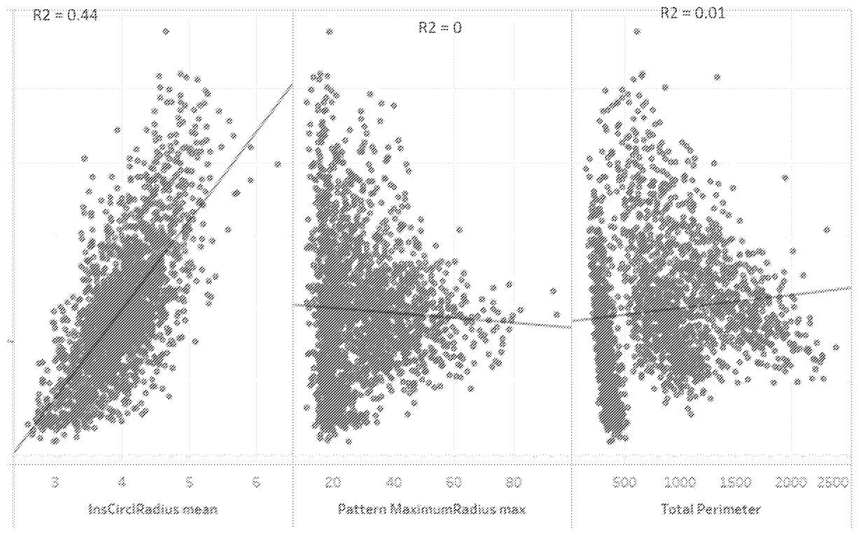

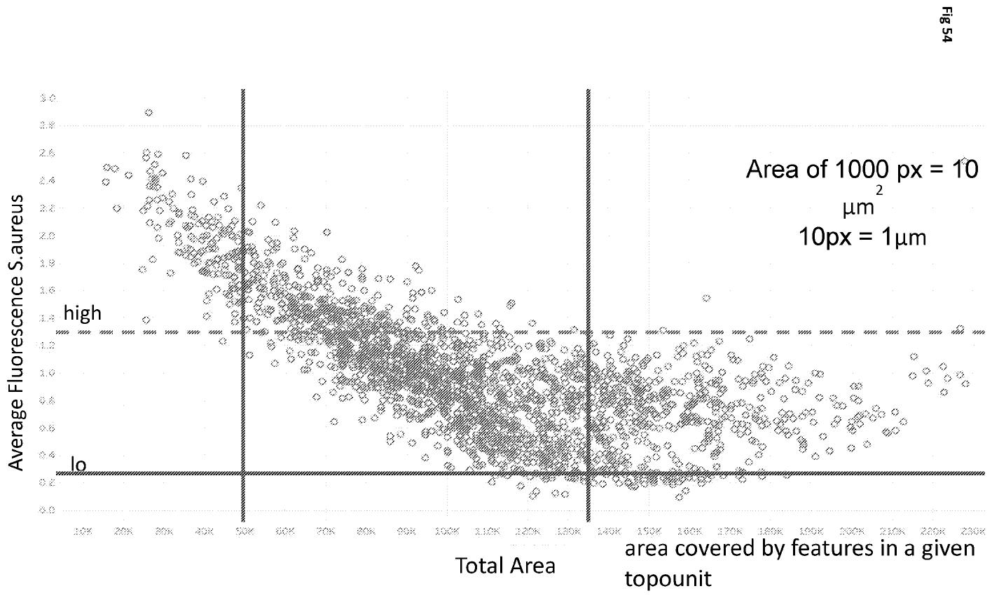

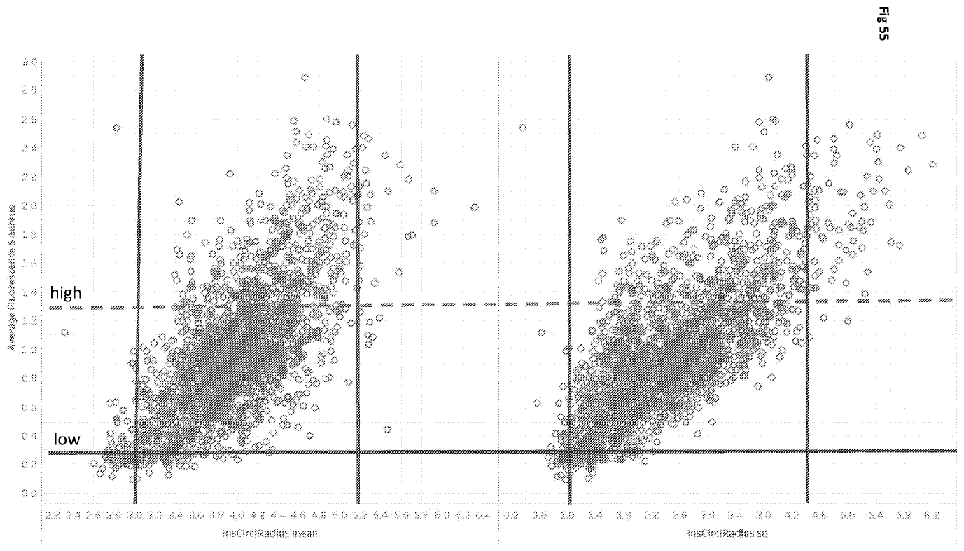

FIG. 53 outlines the important descriptors that can be used for attachment of S. aureus. As shown in FIG. 54 the attachment in relation to the total area can derive a design rule for the descriptor micropillars of Pro-attachment≤500 μm2; Anti-attachment≥1350 μm2. Similarly, as shown in FIG. 55, for inscribed circles Strong anti-attachment topography cannot have radius mean>0.3μm and SD>0.1 μm. FIG. 56 shows attachment in relation to max pattern area, where Medium/low-attachment≥280 μm2 Or less than 30 μm2 depending on values for other descriptors.

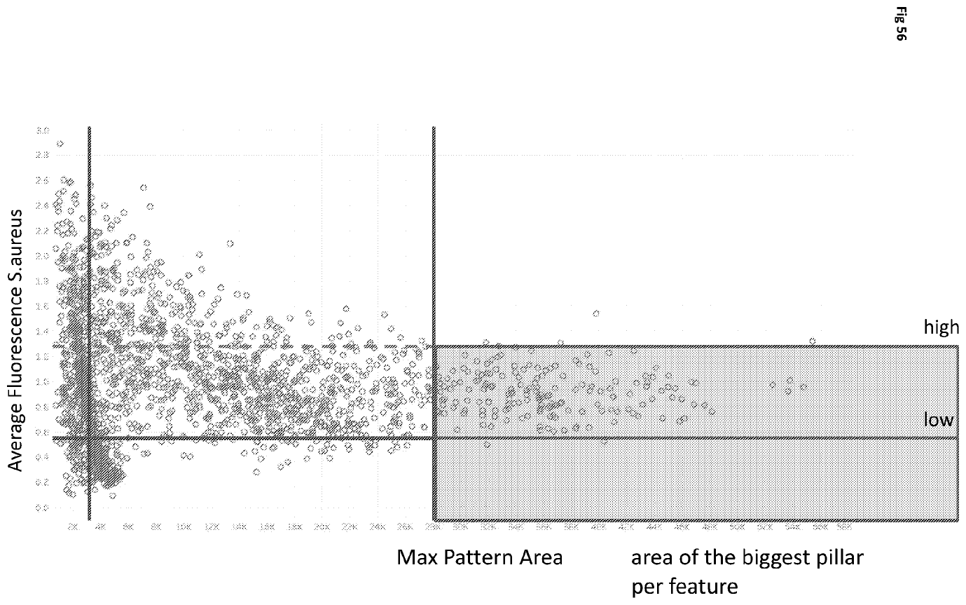

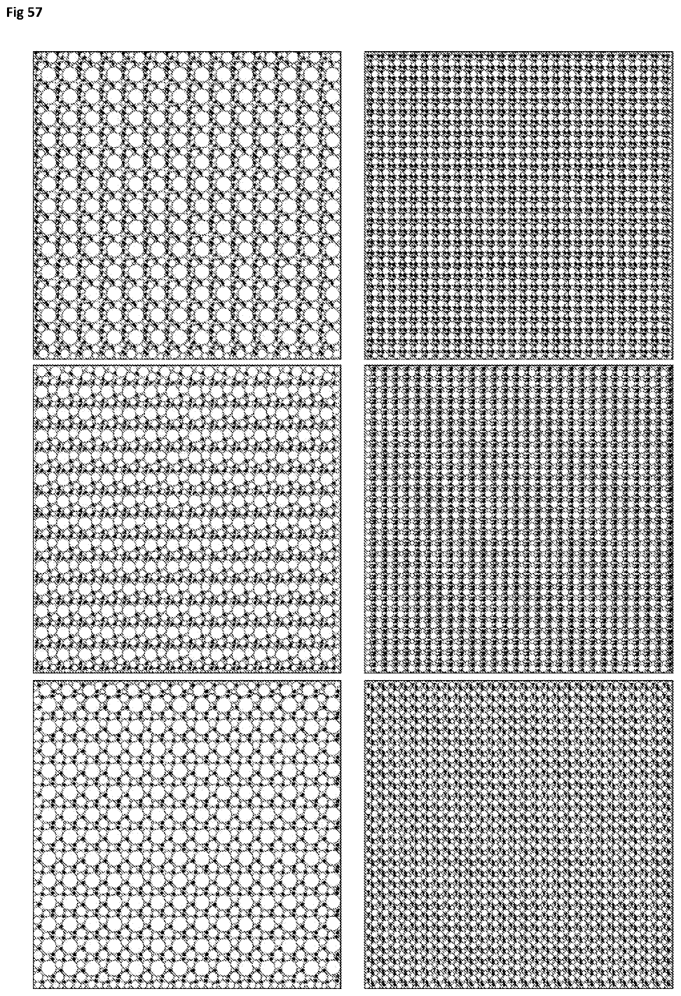

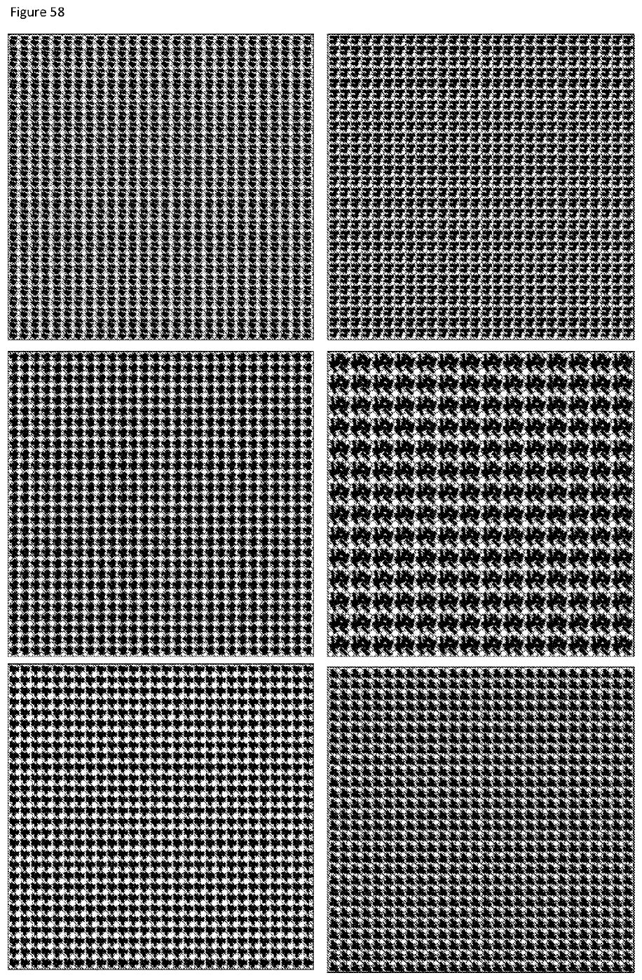

FIG. 57 shows high Attachment Topography Examples. The black elements are the featured topographies; the blue circles are the inscribed circles one can fit between the topographies. Similarly, FIG. 58 shows low attachment topography examples. It can be seen how using the inscribed circle analysis method applied to microtopographical features allows the degree of attachment to be determined and predicted. Smaller inscribed circles are associated with lower attachment topography examples, whilst higher inscribed circles can predict topographical surfaces or combinations that exhibit higher attachment properties.

Further, to determine whether the pro- and anti-attachment properties of the TUs (TopoUnits) were maintained in complex environments where the TU surfaces are likely to be conditioned by host proteins and cells, their response to P. aeruginosa was examined after conditioning with human serum in vitro or after subcutaneous implantation into mice in vivo. In both cases, the anti-attachment properties were maintained suggesting that the deposition of host proteins and cells does not alter the biofilm resistant properties of the anti-attachment TUs. Consequently, these micro-topographies have considerable potential for preventing biofilm formation in a clinical context. This raises the prospect of exploiting micro-topographies to modulate host immune responses and prevent both biofilm-centred infections and prevent foreign body rejection of implanted medical devices.

### REFERENCES

1. Bhat S, Kumar A. Biomaterials and bioengineering tomorrow's healthcare. Biomatter 2013; 3(3):e24717.
2. Holzapfel B M, Reichert J C, Schantz J T, Gbureck U, Rackwitz L, Nöth U, Jakob F, Rudert M, Groll J, Hutmacher D W. How smart do biomaterials need to be? A translational science and clinical point of view. Adv Drug Deliv Rev 2013; 65(4):581-603.
3. Mei Y, Saha K, Bogatyrev S R, Yang J, Hook A L, Kalcioglu Z I, Cho S W, Mitalipova M, Pyzocha N, Rojas F, Van Vliet K J, Davies M C, Alexander M R, Langer R, Jaenisch R, Anderson D G. Combinatorial development of biomaterials for clonal growth of human pluripotent stem cells. Nat Mater 2010; 9(9):768-78.
4. Hook A L, Chang C Y, Yang J, Luckett J, Cockayne A, Atkinson S, Mei Y, Bayston R, Irvine D J, Langer R, Anderson D G, Williams P, Davies M C, Alexander MR. Combinatorial discovery of polymers resistant to bacterial attachment. Nat Biotechnol 2012; 30(9): 868-875.
5. Busscher H J1, van der Mei H C, Subbiandoss G, Jutte P C, van den Dungen J J, Zaat S A, Schultz M J, Grainger D W. Biomaterial-associated infection: locating the finish line in the race for the surface. Sci Transl Med 2012; 4(153):153rv10.
6. Davies D. Understanding biofilm resistance to antibacterial agents. Nat Rev Drug Discov 2003; 2(2):114-22.
7. Zhao L, Chu, PK, Zhang Y, Wu Z. Antibacterial coatings on titanium implants. J Biomed Mater Res B Appl Biomater. 2009; 91(1):470-80.
8. Goodman S B, Yao Z, Keeney M, Yang F. The future of biologic coatings for orthopaedic implants. Biomaterials 2013; 34(13):3174-3183.
9. Swearingen M C, Granger J F, Sullivan A, Stoodley P. Elution of antibiotics from poly(methyl methacrylate) bone cement after extended implantation does not necessarily clear the infection despite susceptibility of the clinical isolates. Pathog Dis 2016; 74(1):ftv103.
10. Graham M V, Cady N C. Nano and microscale topographies for the prevention of bacterial surface fouling. Coatings 2014; 4(1):37-59.
11. Armentano I, Arciola C R, Fortunati E, Ferrari D, Mattioli S, Amoroso C F, Rizzo J, Kenny J M, Imbriani M, Visai L. The interaction of bacteria with engineered nanostructured polymeric materials: a review. ScientificWorldJournal 2014; 2014:410423.
12. Kumar S, Raj S, Sarkar K, Chatterjee K. Engineering a multi-biofunctional composite using poly(ethylenimine) decorated graphene oxide for bone tissue regeneration. Nanoscale 2016; 8(12):6820-36.
13. Schumacher J F, Aldred N, Callow M E, Finlay J A, Callow J A, Clare A S, Brennan A B. Species-specific engineered antifouling topographies: correlations between the settlement of algal zoospores and barnacle cyprids. Biofouling 2007; 23(5-6): 307-17.
14. Schumacher J F, Carman M L, Estes T G, Feinberg A W, Wilson L H, Callow M E, Callow J A, Finlay J A, Brennan A B. Engineered antifouling microtopographies—effect of feature size, geometry, and roughness on settlement of zoospores of the green alga Ulva. Biofouling. 2007; 23(1-2):55-62.
15. Schumacher J F, Long C J, Callow M E, Finlay J A, Callow J A, Brennan A B. Engineered nanoforce gradients for inhibition of settlement (attachment) of swimming algal spores. Langmuir 2008; 24(9):4931-7.
16. Chung K K, Schumacher J F, Sampson E M, Burne R A, Antonelli P J, Brennan A B. Impact of engineered surface microtopography on biofilm formation of Staphylococcus aureus. Biointerphases 2007; 2(2):89-94.
17. Reddy S T, Chung K K, McDaniel C J, Darouiche R O, Landman J, Brennan A B. Micropatterned surfaces for reducing the risk of catheter-associated urinary tract infection: an in vitro study on the effect of sharklet micropatterned surfaces to inhibit bacterial colonization and migration of uropathogenic Escherichia coli. J Endourol 2011; 25(9):1547-52.
18. May R M, Hoffman M G, Sogo M J, Parker A E, O'Toole G A, Brennan A B, Reddy S T. Micro-patterned surfaces reduce bacterial colonization and biofilm formation in vitro: Potential for enhancing endotracheal tube designs. Clin Transl Med 2014; 3:8.
19. Long C J, Schumacher J F, Robinson P A 2nd, Finlay J A, Callow M E, Callow J A, Brennan A B. A model that predicts the attachment behavior of Ulva linza zoospores on surface topography. Biofouling 2010; 26(4):411-9.
20. Unadkat H V, Hulsman M, Cornelissen K, Papenburg B J, Truckenmuller R K, Carpenter A E, Wessling M, Post G F, Uetz M, Reinders M J, Stamatialis D, van Blitterswijk C A, de Boer J. An algorithm-based topographical biomaterials library to instruct cell fate. Proc Natl Acad Sci 2011; 108(40):16565-70.
21. Zhao Y, Truckenmuller R, Levers M, Hua W S, de Boer J, Papenburg B. High-definition micropatterning method for hard, stiff and brittle polymers. Mater Sci Eng C Mater Biol Appl 2017; 71:558-564.
22. Ray S, Shard A G. Quantitative analysis of adsorbed proteins by x-ray photoelectron spectroscopy. Anal Chem 2011; 83(22):8659-66.
23. Percival S L, Suleman L, Vuotto C, Donelli G. Healthcare-associated infections, medical devices and biofilms: risk, tolerance and control. J Med Microbiol 2015; 64:323-34.
24. Popat R, Crusz S A, Messina M, Williams P, West S A, Diggle S P. Quorum-sensing and cheating in bacterial biofilms. Proc Biol Sci 2012; 279(1748):4765-71.
25. Utada A S, Bennett R R, Fong J C N, Gibiansky M L, Yildiz F H, Golestanian R, Wong G C L. Vibrio cholerae use pili and flagella synergistically to effect motility switching and conditional surface attachment. Nat Commun 2014; 5:4913.
26. Therneau T. rpart: Recursive Partitioning and Regression Trees. 2018.
27. R Development Core Team. R: a language and environment for statistical computing. 2014.
28. Hothorn T, Hornik K, Zeileis A. Unbiased recursive partitioning: a conditional inference framework. J Comput Graph Stat 2006; 15(3):651-674.
29. Liaw A., Wiener M. Classification and Regression by randomForest. R News 2002; 2/3:18-22.
30. Kuhn M. Caret: Classification and Regression Training R Package Version 6.0-37. 2014.
31. Rusconi R, Lecuyer S, Guglielmini L, Stone H A. Laminar flow around corners triggers the formation of biofilm streamers. J R Soc Interface 2010; 7(50):1293-9.
32. Whitehead K A, Verran J. The effect of surface topography on the retention of microorganisms. Food Bioprod Process 2006; 84(4):253-259.
33. Shi X, Zhu X. Biofilm formation and food safety in food industries. Trends Food Sci Technol 2009; 20(9):407-413.
34. Ivanova E P, Hasan J, Webb H K, Truong V K, Watson G S, Watson J A, Baulin V A, Pogodin S, Wang J Y, Tobin M J, Labe C, Crawford R J. Natural bactericidal surfaces: mechanical rupture of Pseudomonas aeruginosa cells by cicada wings. Small. 2012; 8(16): 2489-94.
35. Hasan J, Jain S, Padmarajan R, Purighalla S, Sambandamurthy V K, Chatterjee K. Multi-scale surface topography to minimize adherence and viability of nosocomial drug-resistant bacteria. Mater Des. 2018; 140:332-344.
36. Wu S, Zuber F, Maniura-Weber K, Brugger J, Ren Q. Nanostructured surface topographies have an effect on bactericidal activity. J Nanobiotechnology. 2018; 16(1):20.
37. Conrad J C, Gibiansky M L, Jin F, Gordon V D, Motto D A, Mathewson M A, Stopka W G, Zelasko D C, Shrout J D, Wong G C L. Flagella and pili-mediated near-surface single-cell motility mechanisms in P. aeruginosa. Biophys J 2011; 100(7):1608-1616.
38. Mattick J S. Type IV pili and twitching motility. Annu Rev Microbiol 2002; 56:289-314.
39. Haiko J, Westerlund-Wikström B. The role of the bacterial flagellum in adhesion and virulence. Biology (Basel) 2013; 2(4):1242-1267.
40. Cheng Y T, Rodak D E, Wong C A, Hayden C A. Effects of micro- and nano-structures on the self-cleaning behaviour of lotus leaves. Nanotechnology
41. Li X, Cheung G S, Watson G S, Watson J A, Lin S, Schwarzkopf L, Green D W. The nanotipped hairs of gecko skin and biotemplated replicas impair and/or kill pathogenic bacteria with high efficiency. Nanoscale 2016; 8(45):18860-18869.
42. Chebolu A, Laha B, Ghosh M, Nagahanumaiah. Investigation on bacterial adhesion and colonisation resistance over laser-machined micro patterned surfaces. Micro & Nano Letters 2013; 8(6):280-283.

### Example 2—Identification of Microtopographies and Features of Microtopographies which Reproducibly and Predictably Modulate Human Monocytes/Macrophages

A high throughput screening approach was utilised to investigate the relationship between topography and human monocyte-derived macrophage attachment and phenotype, using a diverse library of 2176 micropatterns generated by an algorithm. This reveals that micropillars 5-10 μm in diameter play a dominant role in driving macrophage attachment compared to the many other topographies screened, an observation that chimes with studies of the interaction of macrophages with particles. Combining the pillar size with the micropillar density is found to modulate cell phenotype from pro to anti-inflammatory states. Machine learning was used to successfully build a model that correlates cell attachment and phenotype with a selection of descriptors, illustrating that materials can be designed to induce pro-inflammatory, anti-inflammatory or regulatory immune responses, for future application in the fight against foreign body rejection of medical devices.

### SUMMARY

It is demonstrated for the first time that unbiased screening of an algorithm generated topographical library in combination with machine learning algorithms can be used to identify topographies which promote both the attachment and polarisation of macrophages in the absence of exogenous cytokines. As macrophages are key mediators of inflammatory and tissue repair processes, the ability of surface topography to mediate changes in cell phenotype provides a powerful tool in the goal of achieving rationale design of ‘immune-instructive’ biomaterials for implantable medical devices. Within the context of biomaterials discovery and immune-bioengineering, this offers a defined platform and robust strategy not only for new and novel applications but also understanding the basic biological mechanisms underlying these phenomena.

Macrophage-TopoChip Attachment Screening

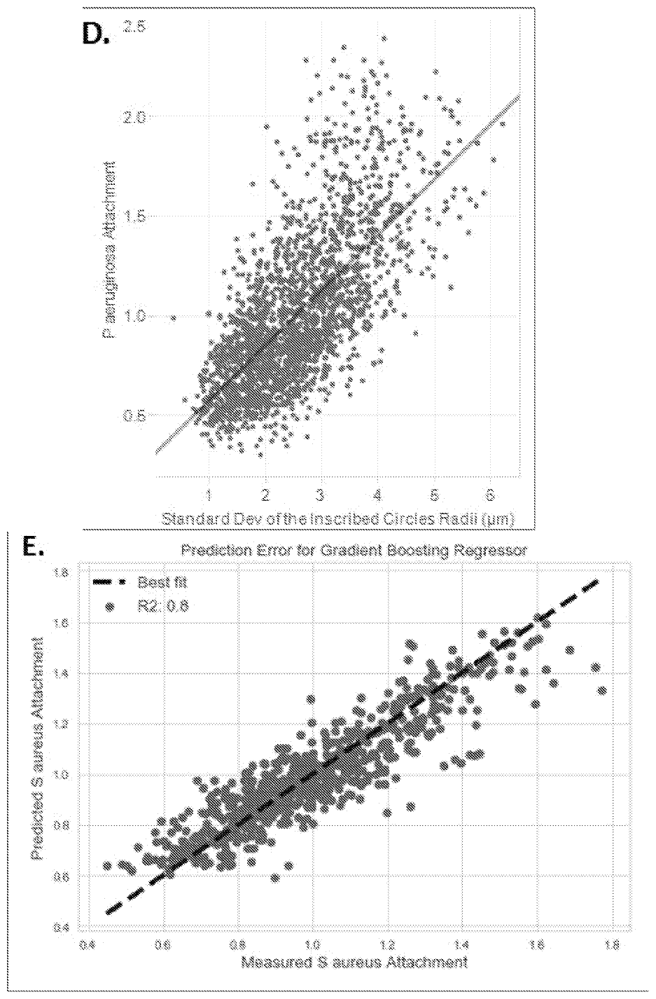

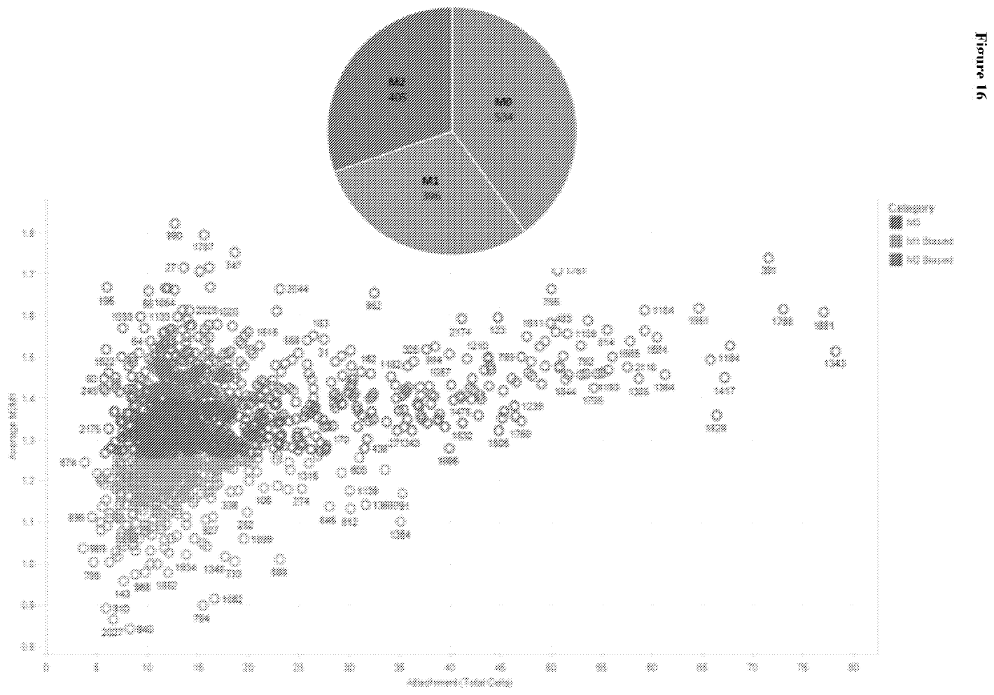

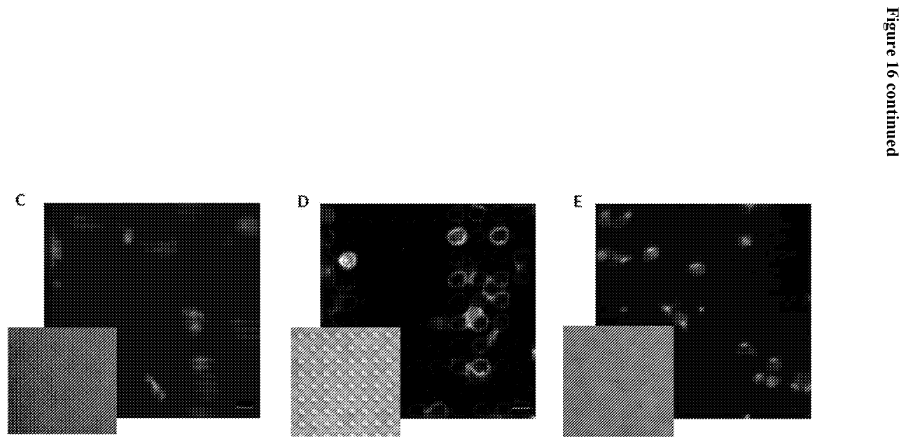

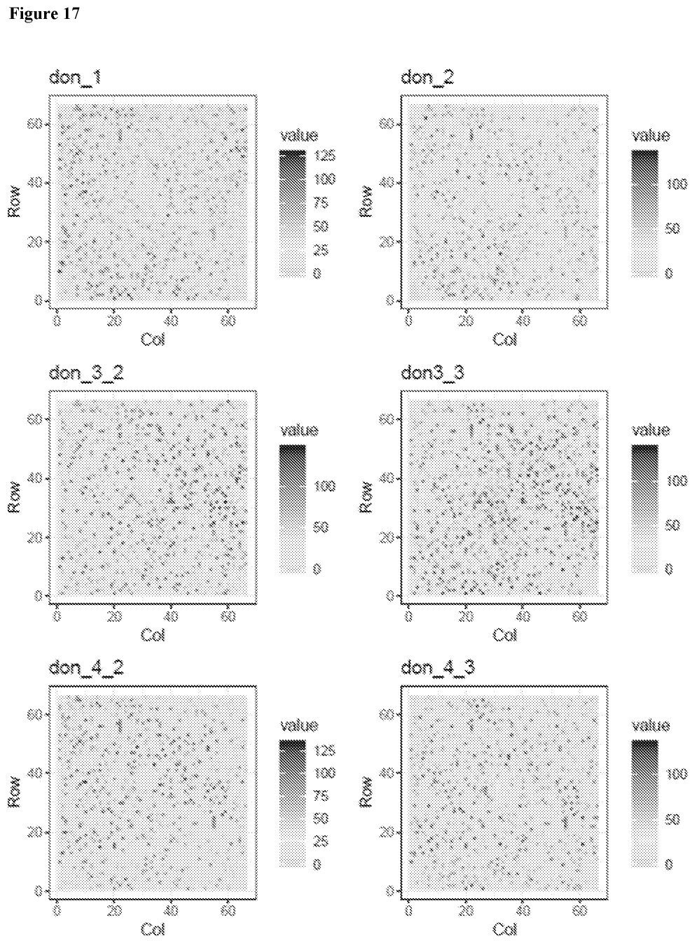

Monocytes were isolated from peripheral blood mononuclear cells (PBMCs) from human blood obtained in the form of buffy coats, using CD14 magnetic beads (Miltenyi Biotec) and used for the TopoChip screening (FIG. 14a). These cells were at no point stimulated or exposed to any exogenous cytokines. Using oxygen plasma etched polystyrene TopoChips in a serum containing media. High throughput screening was carried out using monocytes obtained from five independent donors. Rank order analysis of the cell attachment (FIG. 14b) was compared across the different donors showing consistency for the high and low attachment surfaces (FIG. 17) indicating the attachment measured was statistically robust. The flat, planar surface had a mean attachment of 6 cells per TopoUnit (indicated by blue dotted line; FIG. 14b). Overall, monocyte attachment was significantly higher in the presence of topographical features compared to the flat planar control surface. Amongst the patterned surfaces, there was clear differential attachment of monocytes to specific surface types ranging from over 100 cells (per TopoUnit area) on high attachment TopoUnits compared to less than 10 for low attachment topographies (FIG. 14c).

Characterisation of Polymer Surface Chemistry

In order to understand the role of specific surface features and characterise the differential attachment we employed a computational regression analysis approach. The dataset was pre-processed and aggregated using the mean cell attachment across all donors and TopoUnits with removal of data with a signal to noise ratio (SNR)<2. Subsequently, multiple regression modelling using Gradient Boosting Regression was applied to the data to correlate cellular attachment with a library of 65 specific surface feature descriptors (listed in Supplementary Table 1) generated from a combination of parameter values used to construct the features and parameters generated from image analysis (bright field images) which describe characteristics of surface feature area and shape. The model generated an R² of 0.9 and 0.75 for the macrophage attachment training and test sets respectively (FIGS. 18b & 18c) suggesting the models adequately describe the dataset. Descriptions that highlighted the size of the individual components of the TopoUnits were dominant in the model, specifically the presence of micro pillars with a small surface pattern area (Pattern Area), capturing quantitatively the differences between high and low attachment TopoUnits seen in FIG. 14 topographies. These models are shown to accurately predict the responses to test sets containing combinations of known and novel topographies and show that these surface features have the largest impact on the biological properties. This indicates the potential for using high throughput data sets generated from TopoChip screening to develop robust surface structure-cell response models using machine learning.

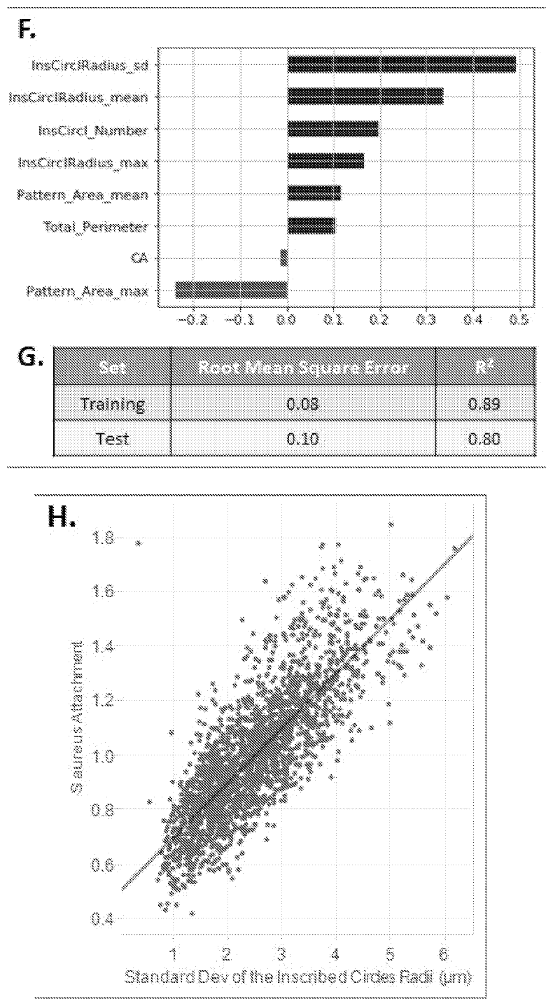

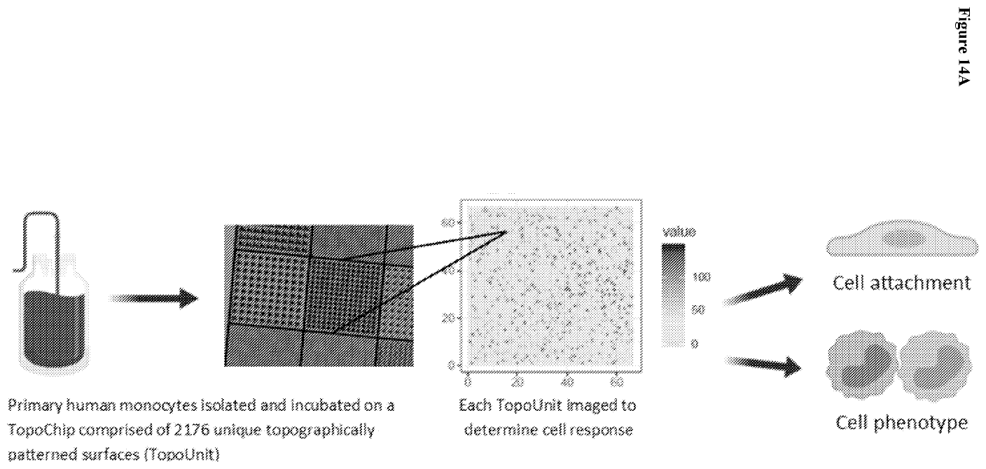

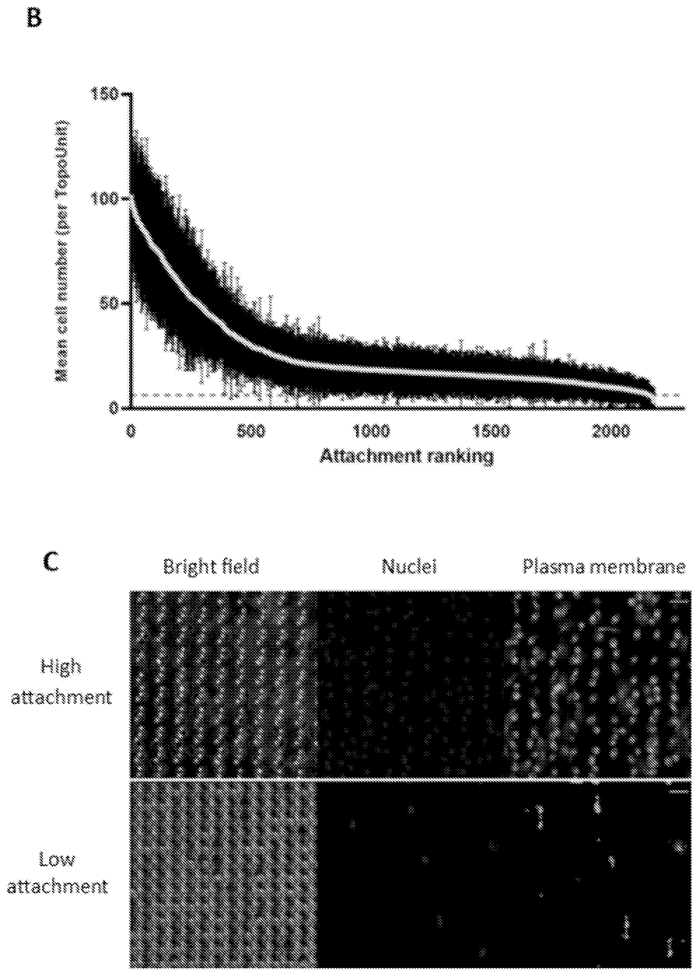

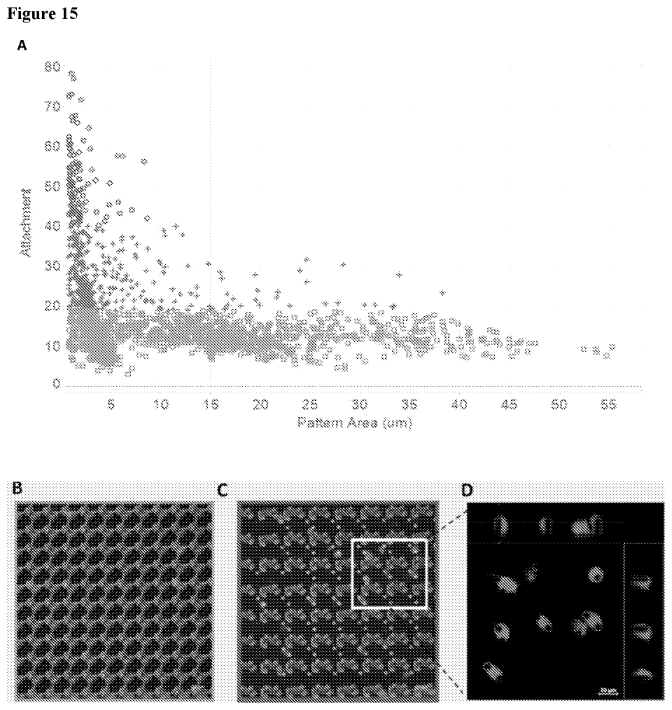

In order to identify the specific physical feature types responsible for macrophage attachment, surface feature importance was calculated and expressed as Shapley Additive exPlanantion (SHAP) values to determine the most importance surface parameter for macrophage attachment. The performance of this model and the descriptors that contributed most strongly to cell attachment are related to the presence of cylindrical micro-pillars in the TopoUnits and a number of associated structural descriptors (see FIG. 18b). To understand the role of micro-pillar size specifically, we clustered the cellular attachment data using K-means (finding three clusters with high (5.5%), medium (20%) and low macrophage attachment (74.5%)) and correlated those groups to TopoUnit performance. The highest attachment of macrophages across the TopoChip was noted on the surfaces which contained micro-pillars of 5 microns in diameter (based on Pattern Area) (FIG. 15a). High attachment was also noted on TopoUnits with micro-pillars up to 10 microns, however, this was the critical size above which macrophage attachment diminished significantly. Confocal imaging of macrophages on high and low attachment TopoUnits indicated specific cell-surface interactions with respect to feature size and cell attachment. On low cell attachment TopoUnits (with surface features>10 μm) the cell adhesion occurred in between the large features (FIG. 15b) in contrast to high attachment surfaces where micro-pillars appeared to be completely engulfed by the macrophages (FIG. 2d). The observation of engulfment of micropillars as the dominant differentiator of attachment in this library is in line with previous observations of macrophage interaction with surfaces and micro particles in this size range.[26]

Differential Macrophage Surface Interaction with Topographies is not Due to Changes in Surface Chemistry

In order to determine if the adsorption of biomolecules was different on different TopoUnits, we characterised the surface of the topographies using in situ mass spectrometry before and after media exposure using a time of flight secondary ion mass spectrometry (ToF SIMS). A selection of high and low attachment TopoUnits were incubated with RPMI media (with 10% foetal bovine serum) for 1 hr or left untreated and subsequently analysed using the 3D SIMS instrument specifically 2D surface chemical imaging (see methods).[27]

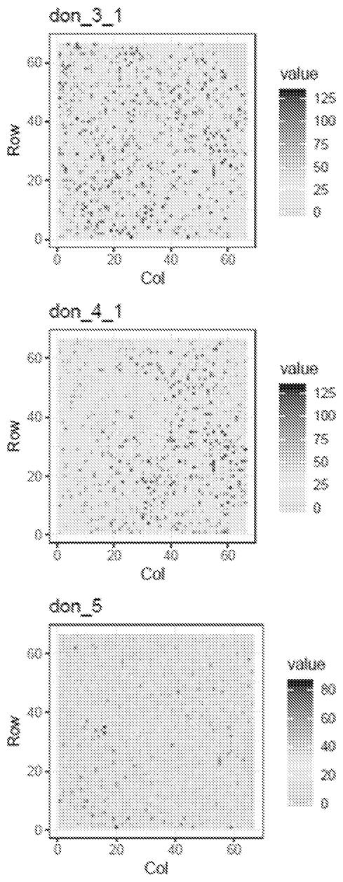

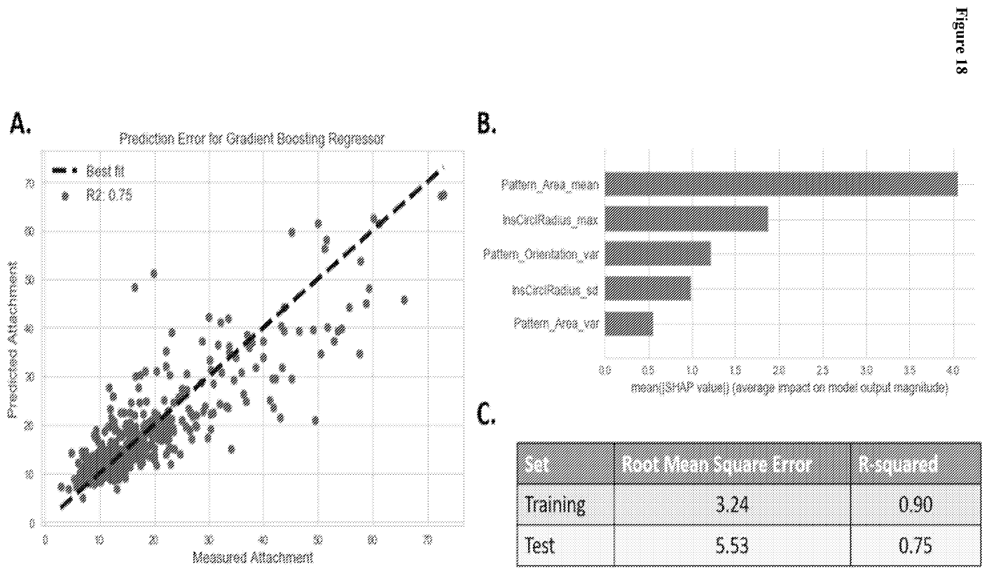

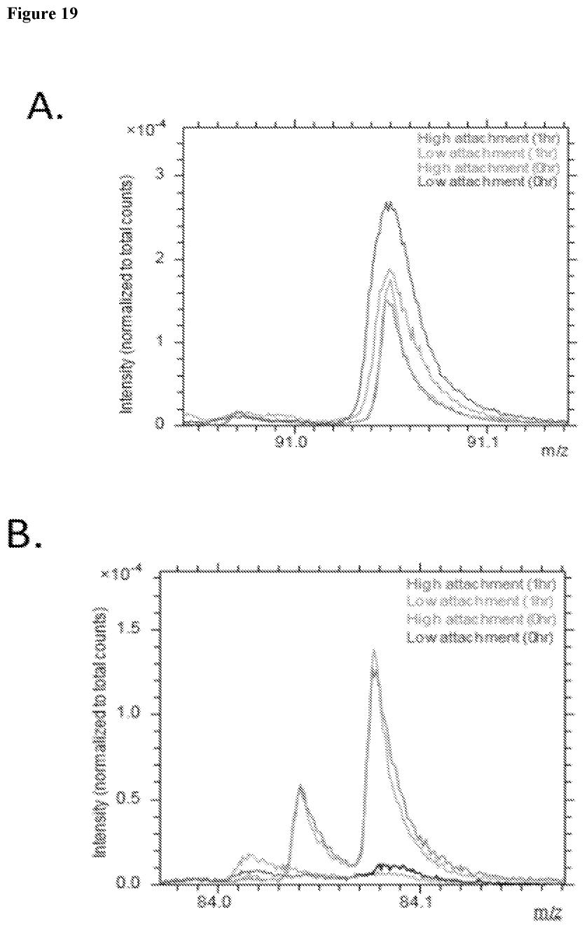

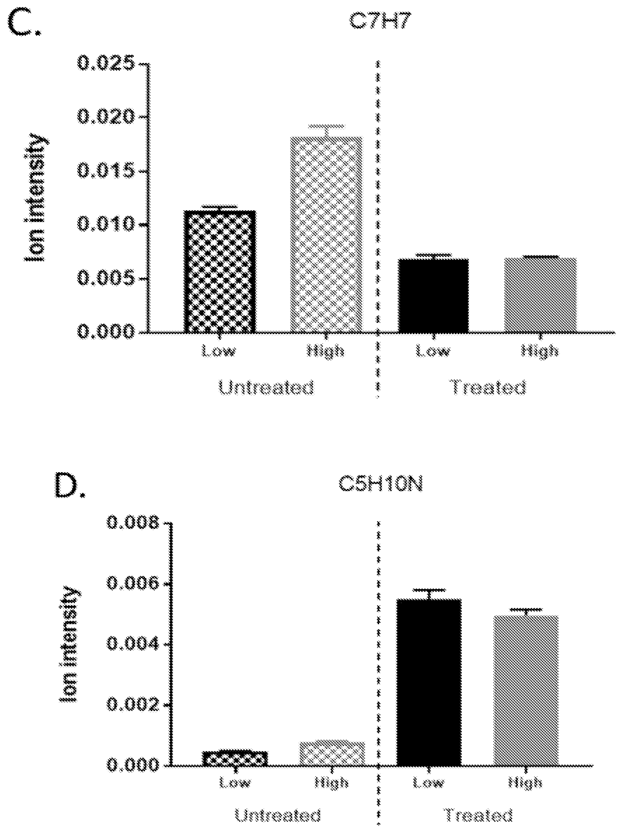

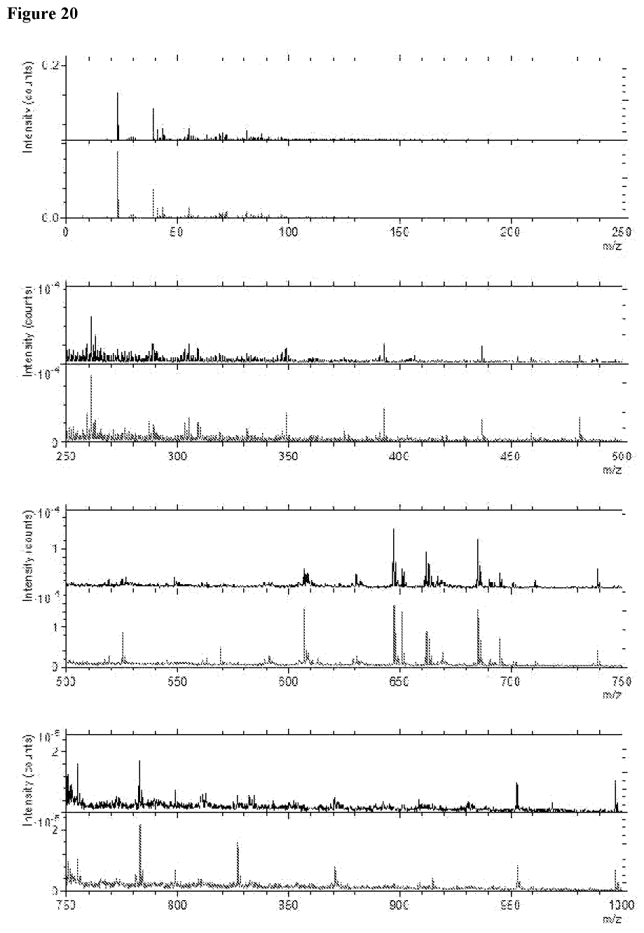

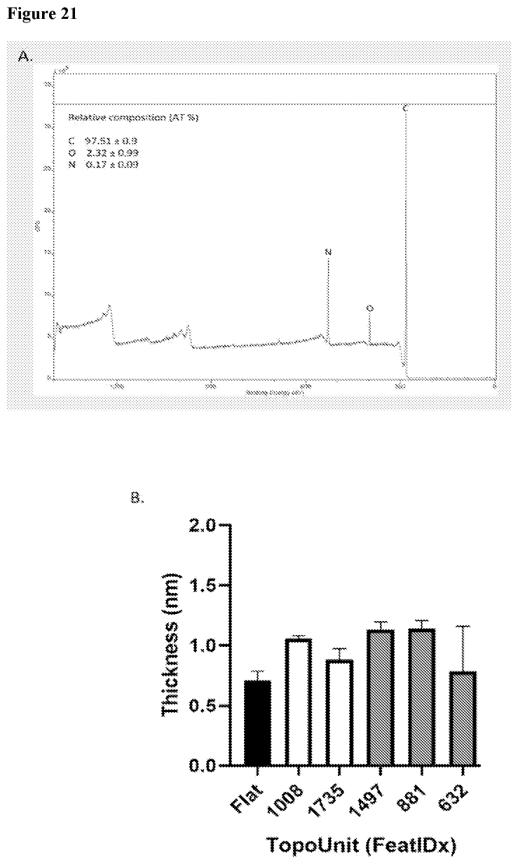

Assessment of the media treated and un-treated TopoUnits 3D SIMS data (FIG. 18a) revealed differences, primarily associated with protein adsorption on the media treated TopoUnits. This is illustrated using the secondary ion peaks m/z 84 (C₅H₁₀N⁺) and 91 (C₇H₇⁺) representing protein (a generic lysine fragment) and the polystyrene base chemistry respectively (FIGS. 19a & b).[28, 29] A significant decrease in polystyrene signal following media incubation and an associated increase in protein coverage of the surface chemistry was observed illustrating the coverage of the substrate with proteins (FIG. 19c & d). Comparison of these secondary ion intensities on representative high and low attachment surfaces was used to determine if differential biomolecule adsorption to TopoUnits was a factor in the cell response. Secondary ion peak intensities of post-media incubation topographies indicated no significant difference in the between high and low attachment surfaces. While SIMS is limited to fingerprint identification of proteins, comparison of the post incubation spectra suggests that no large compositional differences are observed between the different TopoUnits (FIG. 20). The total protein amount was quantified using X-ray photoelectron spectroscopic (XPS) analysis indicating that there is a strongly adsorbed protein layer of ca. 1 nm thick (dehydrated) which exhibited no correlation with macrophage cell attachment (FIG. 21). To probe the ion coverage and distribution of the selected topographies we used high resolution imaging (utilising delayed extraction of the mass analyser) which showed a uniform surface distribution of the peak m/z 42 (CNO⁻) (unspecific protein marker) in the media incubated samples (FIG. 22). Protein deposition was observed across both high and low attachment surfaces, however, there was no discernible difference in the spatial distribution in terms of the apical, lateral or basal surface of the TopoUnit surfaces. Using the complementary high spatial resolution and chemical characterisation of the surface chemistry provides confidence in assigning the cell response driver to the topography rather than changes in surface chemistry for these micro patterned surfaces.

Modulation of Macrophage Phenotype by Surface Topography

After screening a range of topographies for monocyte attachment and gaining insight into the structure-function relationship, we investigated the influence of surface topography on macrophage phenotype. Polarisation of naïve (M0) macrophages to pro (M1) or anti-inflammatory (M2) phenotypes is a key determinant in maintaining tissue homeostasis after injury and is known to correlate with clinical outcome of implanted medical devices. Harnessing macrophage polarity presents a unique opportunity to control inflammation, prevent rejection and accelerate integration of biomaterials and medical devices. We hypothesised that the surface topography would play a key role in this biological process.

In order to investigate this, monocytes were incubated on TopoChips in the absence of exogenous cytokine stimulation for 6 days prior to phenotypic characterisation. Macrophage phenotypic status was determined using cell surface markers known to be associated with M1/M2 phenotypes (calprotectin and mannose receptor for M1 and M2, respectively).[16] In order to determine phenotypic responses, the M2/M1 ratio was calculated (per cell) and normalised to the flat, planar TopoUnit on each chip respectively. Those TopoUnits with a signal: noise ratio (SNR) of <2 were removed from further analysis.

Overall, the proportion of the three potential phenotypes (M2/M0/M1) across the TopoChip indicated there was a range of phenotypic responses to different topographies, and no one predominant macrophage polarisation status (FIG. 16a). Furthermore, comparison of the phenotype M2/M1 ratio relative to cell attachment did not indicate a strong linear relationship, however, the cluster analysis identified a relationship between cell number per TopoUnit and M2 polarisation status (FIG. 16b). Modulation of macrophage phenotype was reflected by clear changes in surface marker ratios ranging from 1.82-0.8 in FIG. 16c-e (flat planar surface=1.2).

In the same way as for macrophage attachment, we developed a model to describe the macrophage phenotype relative to the surface parameter descriptors to provide information on relevant physical surface structure descriptors. As cell attachment and polarisation were both important factors, we trained machine learning models to predict a composite dependent variable that incorporated both phenotype and attachment: log(M2/M1)×cell attachment. This variable has large positive or negative values to enable identification of TopoUnits with high attachment and a specific phenotype (M2 or MD and low values for those with low attachment/phenotype. Therefore, the units of most interest exhibit either the most positive value for the composite variable to the anti-inflammatory phenotype class (M2), or the materials with most negative values for the composite variable into the pro-inflammatory class (M1). The anti- and pro-inflammatory groups were defined after clustering the dataset and selecting those instances from the clusters with the highest and lowest values found for the composite variable.

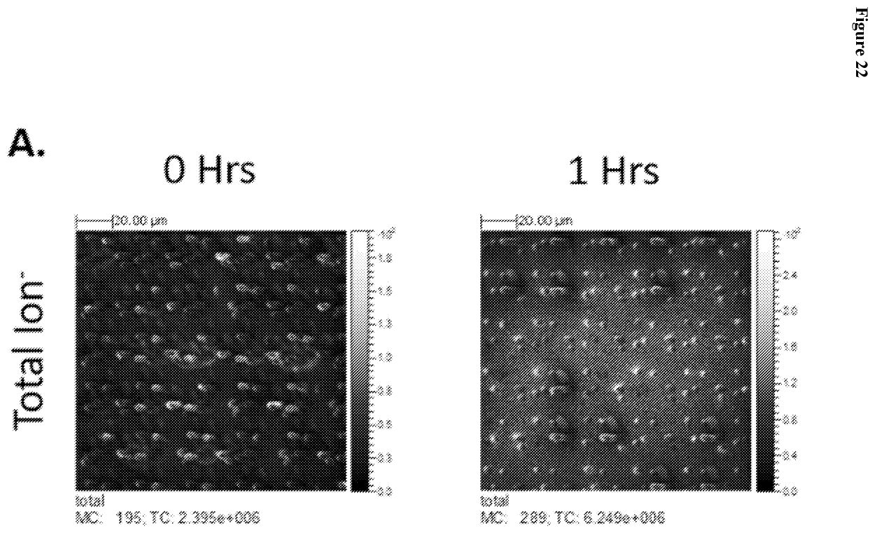

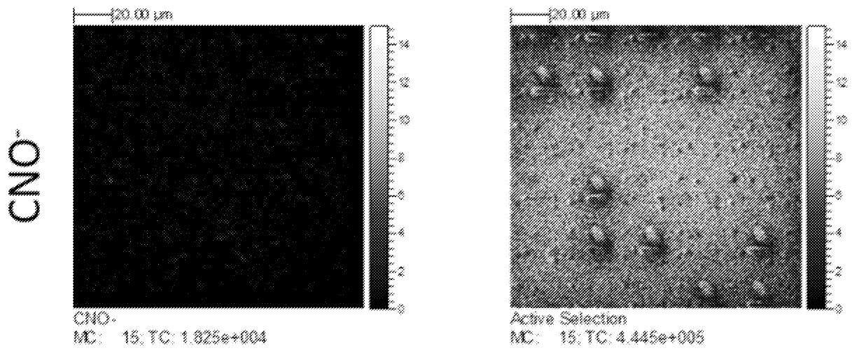

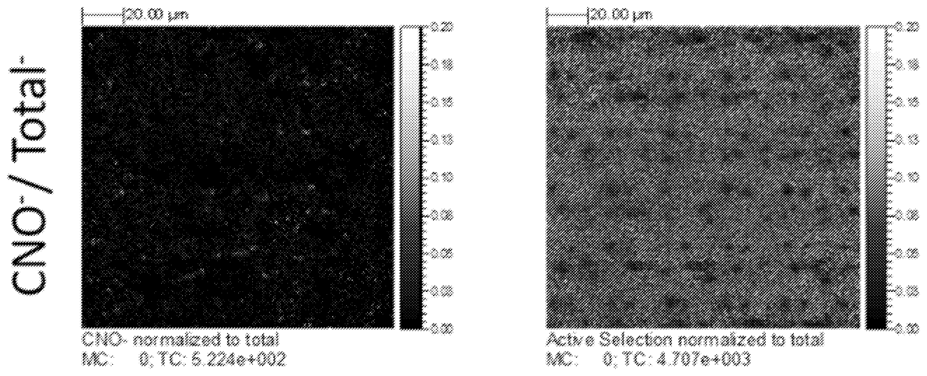

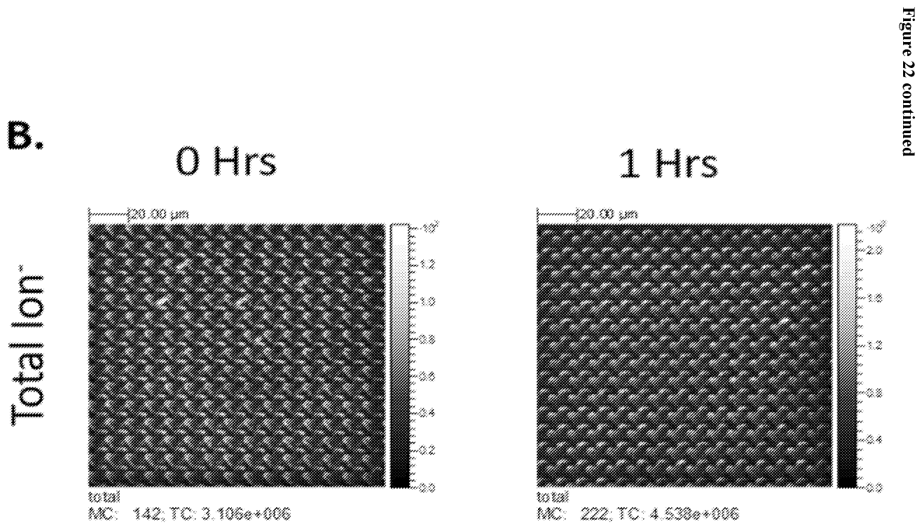

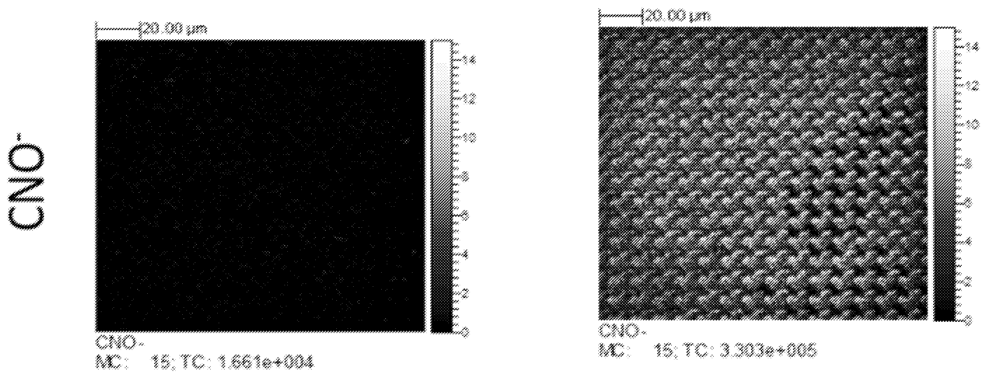

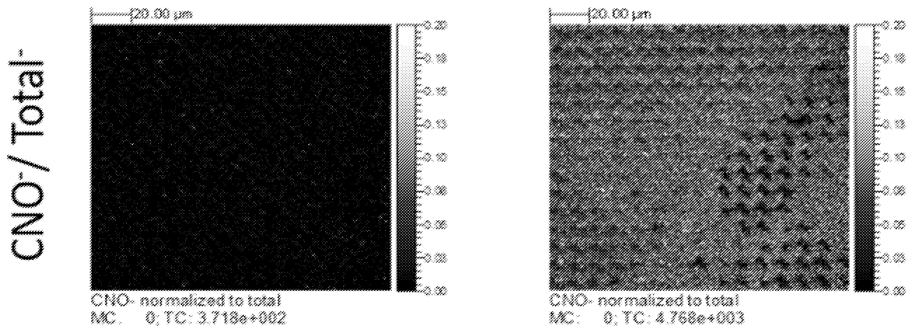

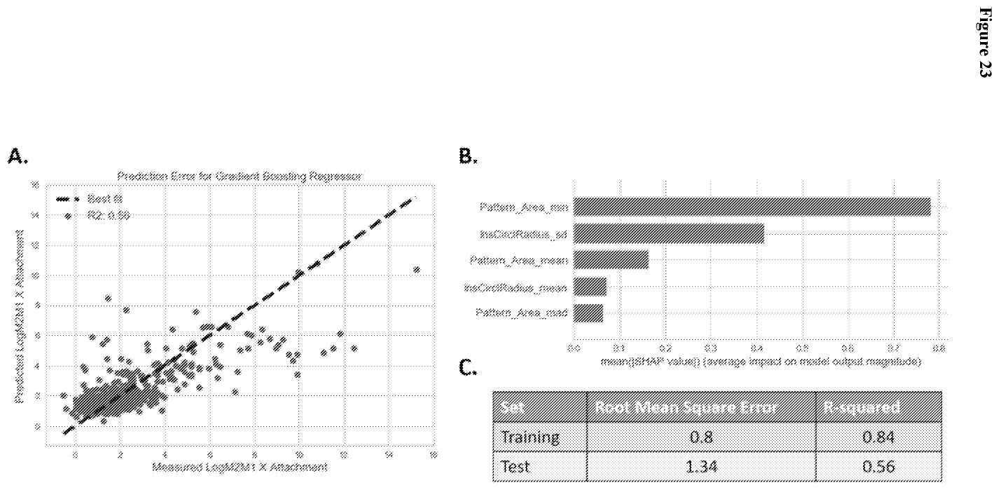

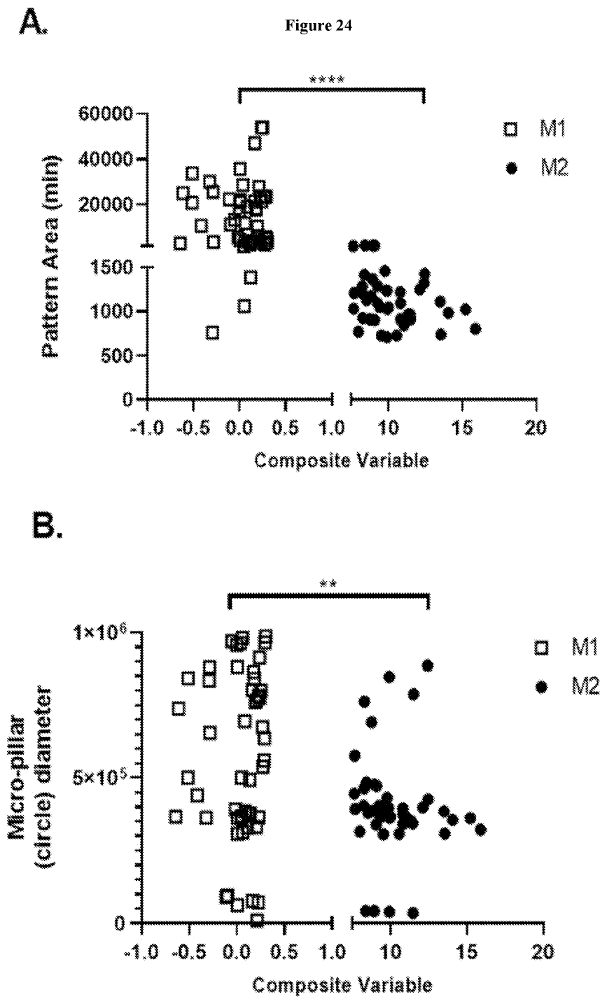

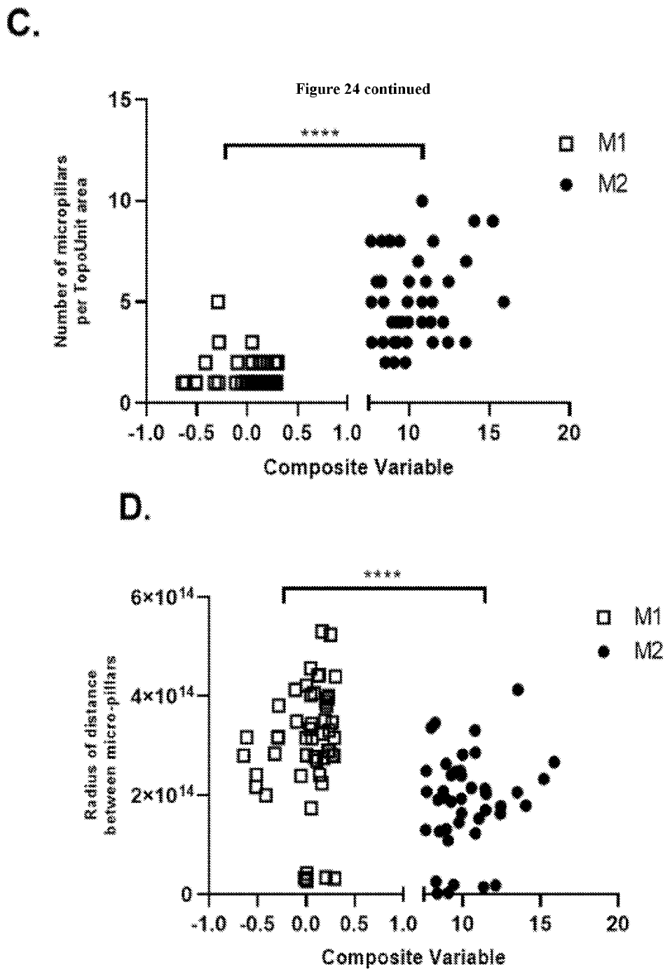

The regression model for polarisation generated an R² of 0.84 and 0.56 for the macrophage phenotype training and test sets respectively, and SHAP values indicated key surface parameters that drive macrophage phenotype modulation (FIG. 23b & c). Specifically, the features associated with phenotypic changes related to feature size described by Pattern Area and most dominantly Pattern Area_min, the smallest in the TopoUnit. The spacing between features (described using MaxInscribedCircles) as a function of micro-pillar density was also a prominent driver of phenotype. Further correlative analysis of the top 50 M2 or M1 TopoUnits showed that these features were all statistically significant in their ability to modulate a specific macrophage phenotype (FIG. 24a-d). Interestingly, it is the combination of these key surface structures that is responsible for driving changes in macrophage phenotype. This is reflected in the representative (inset) bright field microscopy images of TopoUnits in FIG. 16 (c-e) whereby M1 phenotype is driven by larger, more disperse surface features compared to smaller, denser micro-pillars driving an M2 phenotype. Our findings expand on previous work by Bartneck et al. whose results conclude that macrophages cultured on small cylindrical posts (20 μm diameter) showed a high M2 and low M1 surface marker profile, compared to cells on micro patterned grooves which showed increased expression of surface markers characteristic of M1 (pro-inflammatory) status.[26] However, by screening thousands of topographies to sample a wide design space and using hundreds of descriptors, we have been able to determine the importance of micro-pillars compared to a wide range of other shapes and identify the importance of their size and density in the control macrophage behaviour.

The observations noted here are in line with studies focused on the dependence of macrophage phagocytosis on shape and size of microparticles in 3D. Champion et al. reported that shape, more specifically, the localised shape at the point of initial contact determines whether macrophages initiate phagocytosis or simply spread on particles.^{[30-32]}

Discussion

| Biological Outcome | Descriptors | % |
|---|---|---|
| High Macrophage | Inscribed Circle Radius Max | 9 |
| Attachment | Inscribed Circle Standard Deviation | 11 |
| | Pattern Orientation Variance | 8 |
| Low Macrophage | Pattern Area Mean | 24 |
| Attachment | Pattern Area Variance | 17 |
| High M2 Bias | Pattern Area Mean | 17 |
| | Inscribed Circle Radius Standard | 10 |
| | Deviation | |
| Low M2 Bias | Pattern Area Min | 30 |

These descriptors are highlighted by mathematical and machine learning approaches to the dataset. Those approaches also allow the understanding of whether the descriptors impact the biological activity in a positive or negative manner.

The dominant descriptors are listed in the table indicating the pattern area and the spaces between are notable in controlling cell response through the Pattern area Mean/Min and the inscribed circle radius/standard deviation descriptors respectively.

Topographical descriptors are a set of structural properties and characteristics that describe the topographical surface of the materials. For instance, if there is a material with round pillars in the chip, examples of descriptors would be: number of pillars, size of individual pillar, space between the pillars etc.

In the above table the % indicates the percentage (variance) of the whole dataset that can be explained by a particular descriptor.

### REFERENCES

[16] H. M. Rostam, S. Singh, F. Salazar, P. Magennis, A. Hook, T. Singh, N. E. Vrana, M. R. Alexander, A. M. Ghaemmaghami, Immunobiology 2016, 221, 1237.
[21] H. V. Unadkat, M. Hulsman, K. Cornelissen, B. J. Papenburg, R. K. Truckenmuller, A. E. Carpenter, M. Wessling, G. F. Post, M. Uetz, M. J. Reinders, D. Stamatialis, C. A. van Blitterswijk, J. de Boer, Proc Natl Acad Sci USA 2011, 108, 16565.
Walko, C. Chiappini, J. de Boer, F. M. Watt, Acta Biomater 2019, 84, 133.
[26] M. Bartneck, V. A. Schulte, N. E. Paul, M. Diez, M. C. Lensen, G. Zwadlo-Klarwasser, Acta Biomater 2010, 6, 3864.
[27] M. K. Passarelli, A. Pirkl, R. Moellers, D. Grinfeld, F. Kollmer, R. Havelund, C. F. Newman, P. S. Marshall, H. Arlinghaus, M. R. Alexander, A. West, S. Horning, E. Niehuis, A. Makarov, C. T. Dollery, I. S. Gilmore, Nat Methods 2017, 14, 1175.
[28] J. Bailey, R. Havelund, A. G. Shard, I. S. Gilmore, M. R. Alexander, J. S. Sharp, D. J. Scurr, ACS Appl Mater Interfaces 2015, 7, 2654.
[29] J. B. Lhoest, M. S. Wagner, C. D. Tidwell, D. G. Castner, J Biomed Mater Res 2001, 57, 432.
[30] J. A. Champion, S. Mitragotri, Proc Natl Acad Sci USA 2006, 103, 4930.
[31] J. A. Champion, S. Mitragotri, Pharm Res 2009, 26, 244.
[32] J. A. Champion, A. Walker, S. Mitragotri, Pharm Res 2008, 25, 1815.
[33] H. M. Rostam, P. M. Reynolds, M. R. Alexander, N. Gadegaard, A. M. Ghaemmaghami, Sci Rep 2017, 7, 3521.
[34] Y. Zhao, R. Truckenmuller, M. Levers, W. S. Hua, J. de Boer, B. Papenburg, Mater Sci Eng C Mater Biol Appl 2017, 71, 558.
[35] S. M. Lundberg, S. I. Lee, Adv Neur In 2017, 30.
[36] L. Breiman, Mach Learn 2001, 45, 5.
[37] T. Q. Chen, C. Guestrin, Kdd'16: Proceedings of the 22nd Acm Sigkdd International Conference on Knowledge Discovery and Data Mining 2016, 785.

### Example 3 Identification of Microtopographies and Features of Microtopographies which Reproducibly and Predictably Modulate Human Dendritic Cells

Motility of Immature Dendritic Cells on Topographic Features

To determine the effect of topography on dendritic cell movement, the speed of cells moving on top of different features in comparison to a flat PS surface were assessed. Cells were fluorescently labelled as described in 7.1.4 to visualise the cytoplasm and nucleus of dendritic cells.

After live cell imaging the fluorescently labelled cells for 3 hours the images were stitched together in the ImageJ-Fiji program and analysed by the TrackMate PlugIn. With the help of the PlugIn in the stitched together images the movement tracks of the seeded cells were formed and the cell movement quantified. Non-moving cells (that have not moved in 3 hours=wriggling in one spot, movement<10 um) were removed from the analysis. From this analysis nine ‘hit’ topographies have been identified to significantly slow down DC motility and reduce length of travel in comparison to the on-chip flat surface control (p<0.001) (FIGS. 25 and 26).

Correlation the values of mean speed and track displacement in the different topography units gives a good correlation of R2 of 0.7821.

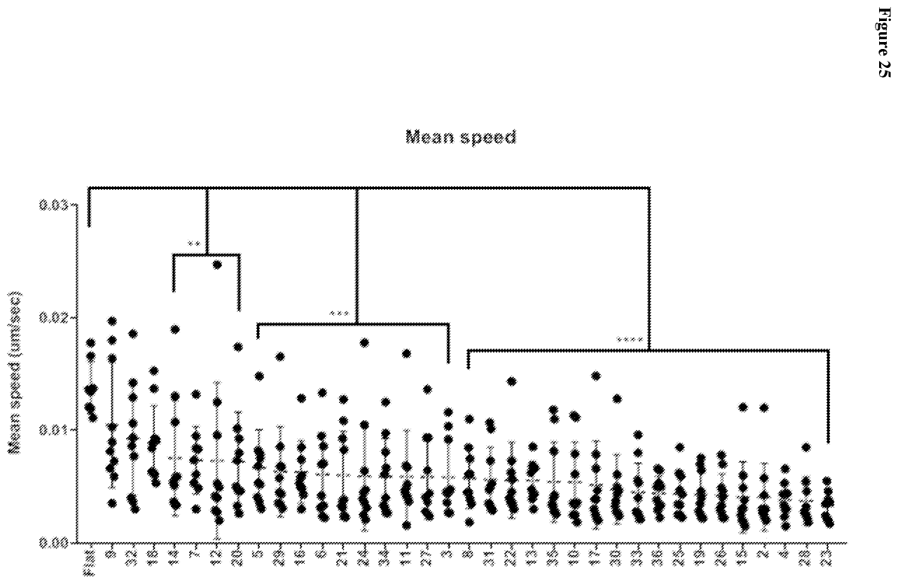

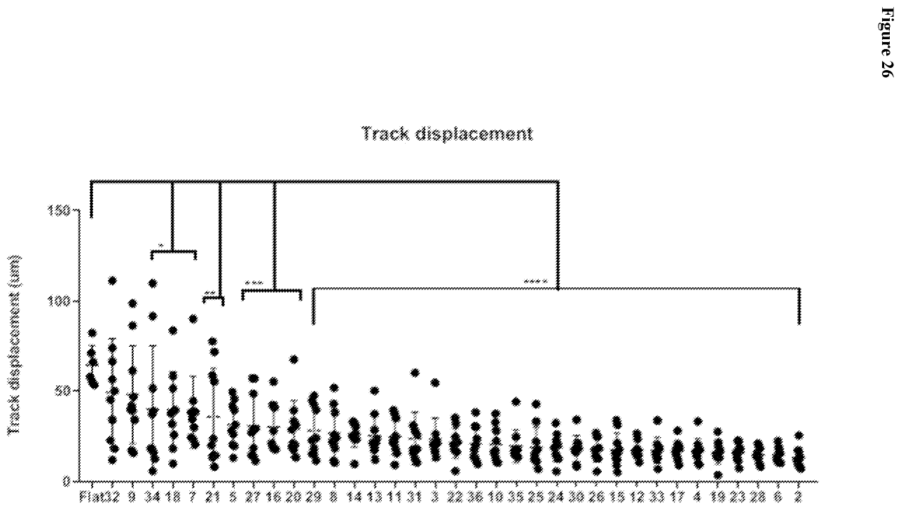

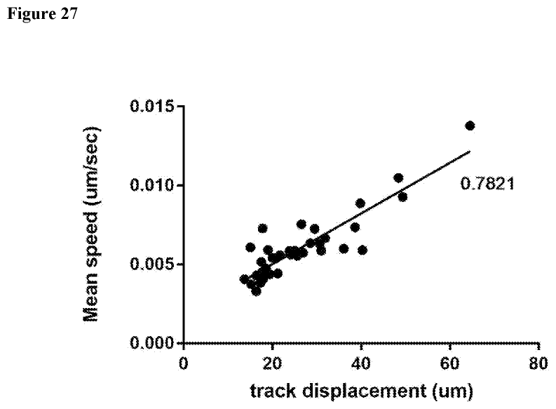

Analysing the topographic features of ‘hit’ topographies has shown the spacing of ‘fast’ features to be more distant and more complex in comparison to the five ‘slowest’ topographic features (FIGS. 27 and 28).

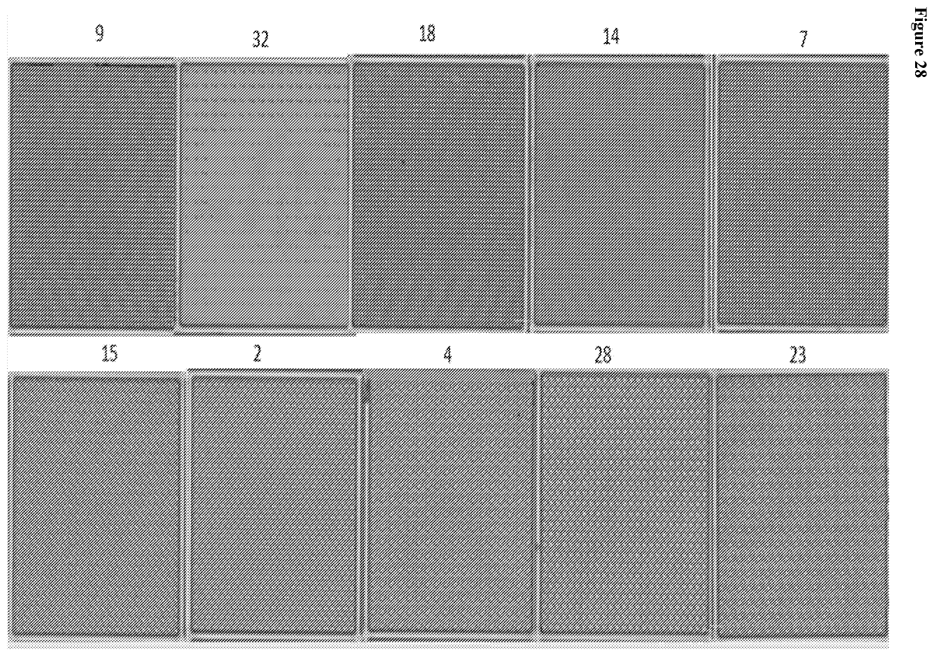

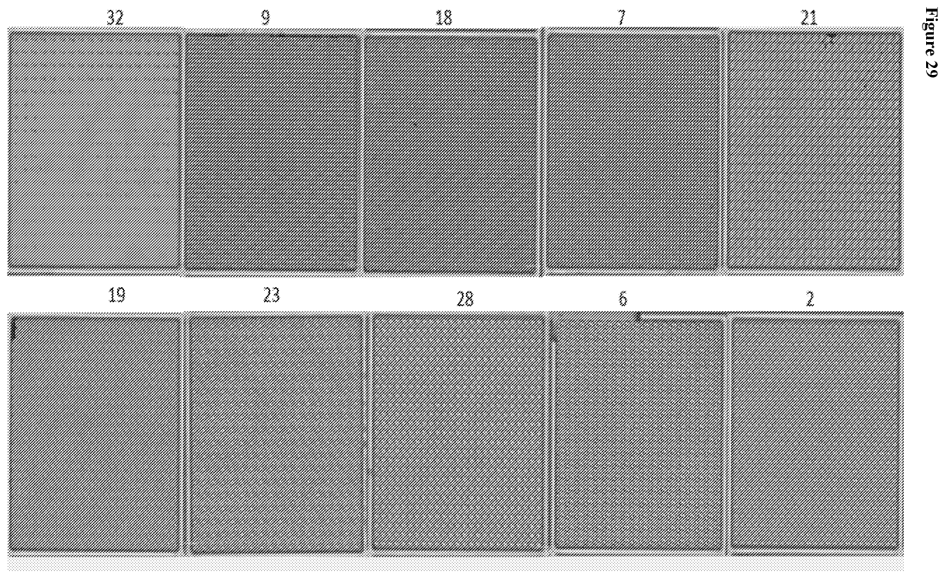

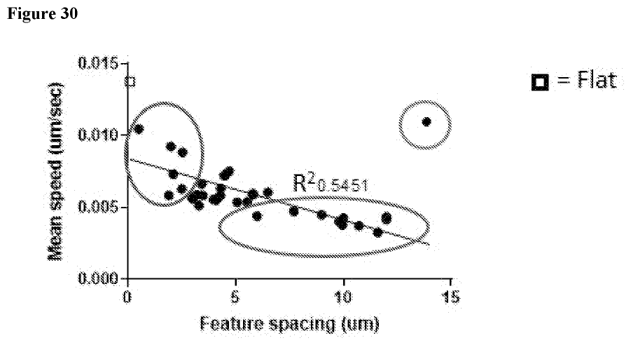

Looking at the topographies portrayed in FIGS. 28 and 29 a pattern seemed to form. Therefore we correlated the mean speed cells moved on those topographies with the diagonal spacing in between topographic features (see FIG. 30). Different groups can easily be classified with this graph and good correlation of R2 of 0.5451.

These results show the general trends:

Features that are very close to each other (‘little spacing’) have similar movement behaviour as flat, because cells will mostly move on top of the features left circle Features that have some spacing (‘intermediate spacing’) slow down movement and also restrict how far cells will move in between them: cells are going through a ‘labyrinth’ middle circle. Features that are spaced very far apart (‘far spaced’), let cells move freely in between them, cells move unrestricted similar to flat surface right circle. Examples of these are shown in FIG. 31.

Topography Culture Decreased HLA-DR Expression on Immature DCs

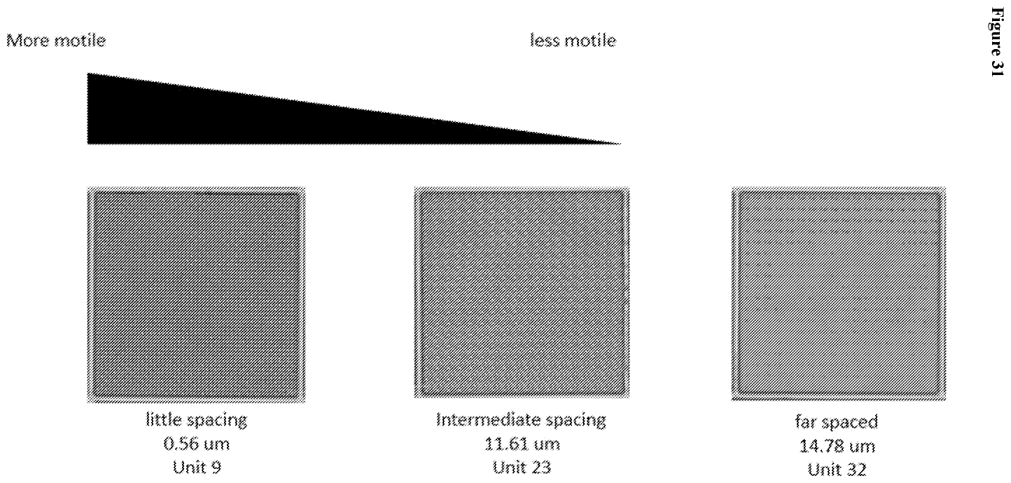

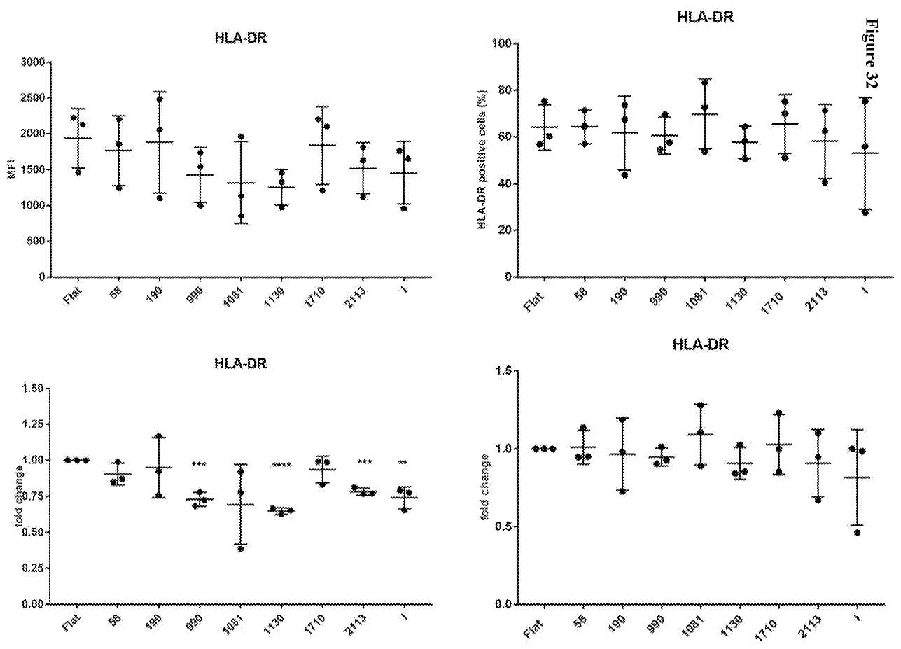

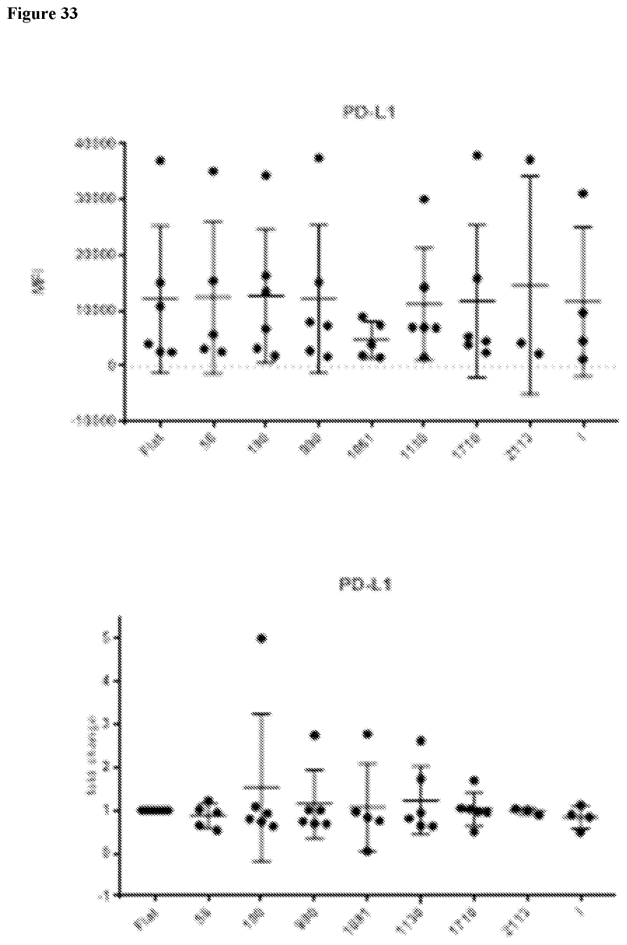

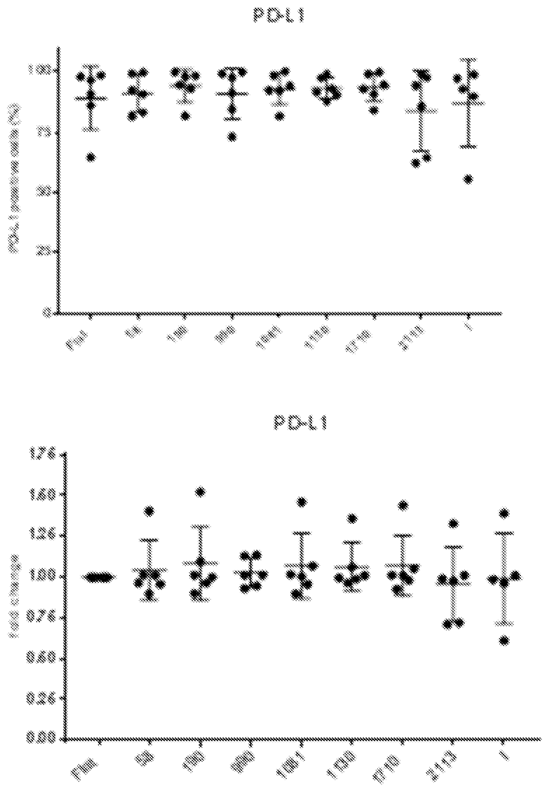

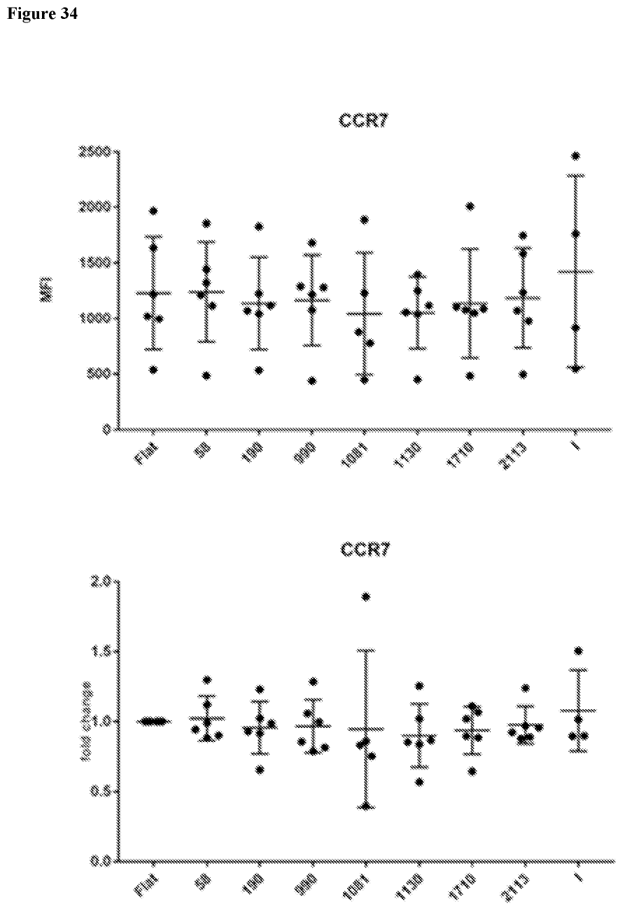

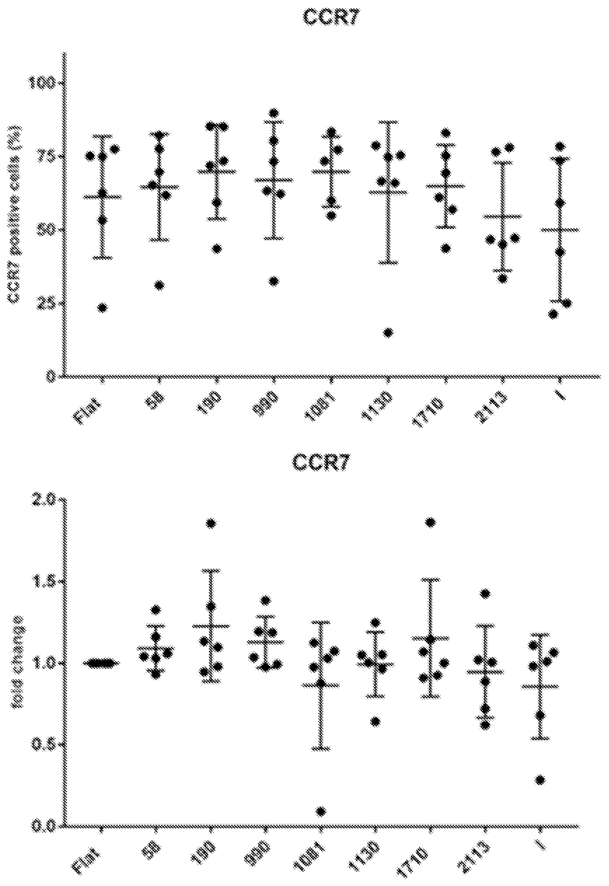

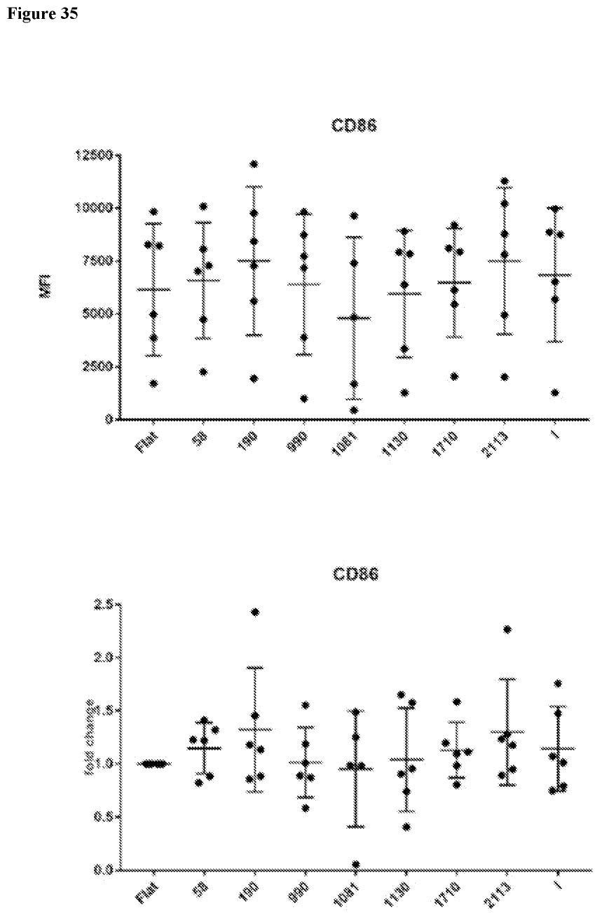

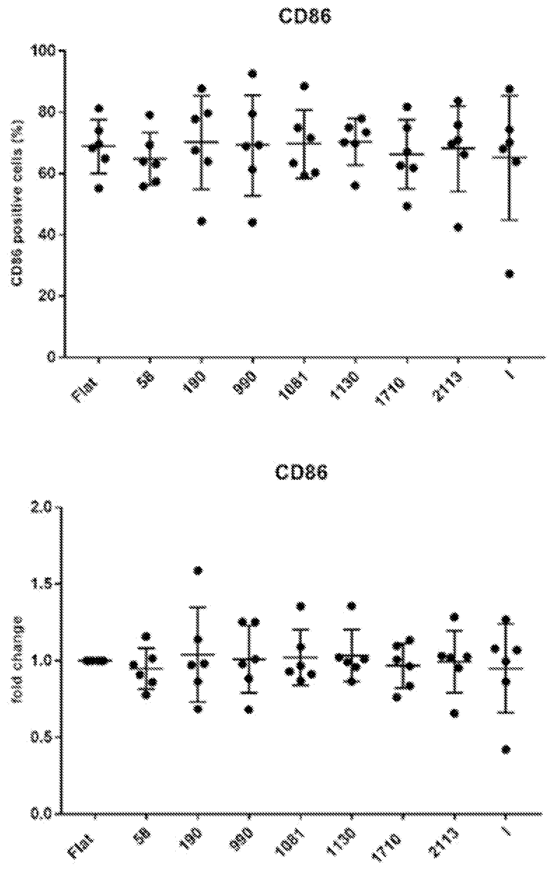

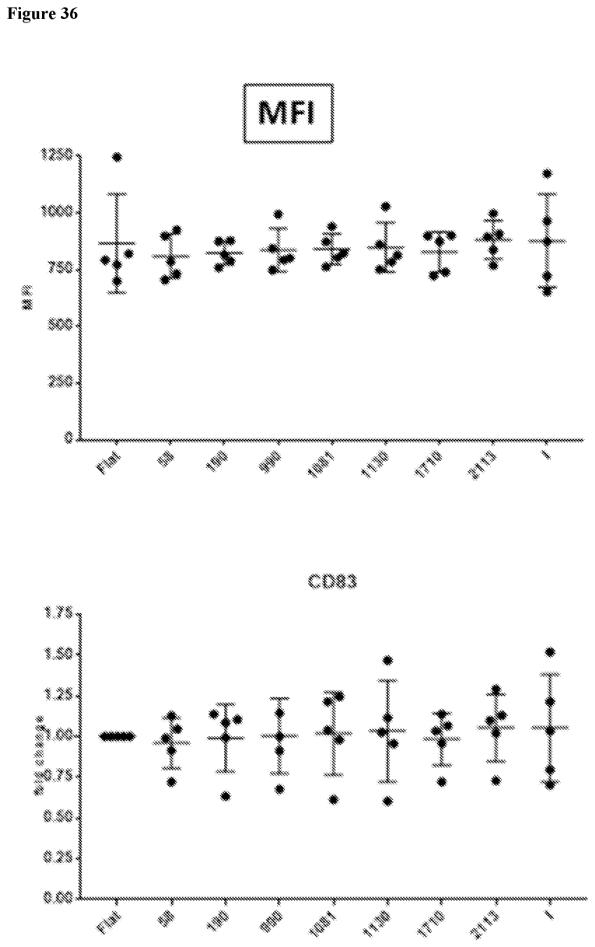

To assess the impact of topographic features on the phenotype of dendritic cells we cultured 6.5×10\hat{ }5 immature DCs for 24 hours on the punched out wafers as described in 7.1.3. Following this topography culture, the phenotype of DCs we did not find significant modulations of the expression levels of investigated surface markers CD83, CD86, CCR7 and PD-L1 (see FIGS. 33-36). Only HLA-DR was shown to be modulated in expression level strength (FIG. 32), with several features seeming to suppress HLA-DR expression—when looking at the fold changes in between flat surface to topographic condition.

LPS Stimulation DCs on Topographies Leads to Less Activated DCs

Seeing how HLA-DR expression is decreased on immature DCs after topography culture, we asked whether topographies might have an effect on the stimulation of TLRs on DCs. In this experimental set-up we used LPS (E. coli) as a model to engage TLR4.

Immature DCs were cultured for 6 hours on the topographic wafers, and then stimulated with LPS for further 18 hours—we then assessed again the expression levels of CD83, CD86, HLA-DR, CCR7 and PD-L1. HLA-DR again was observed to be decreased after culture on two specific topographies (990 and 1130), a level of 20% reduction compared to flat polystyrene (FIG. 37). Interestingly one topography increased HLA-DR expression slightly, but significantly (1081).

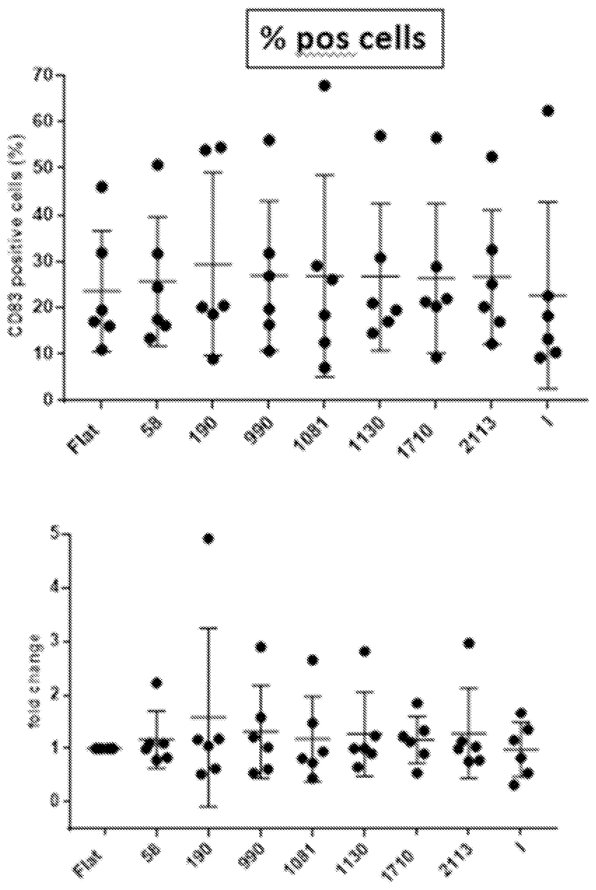

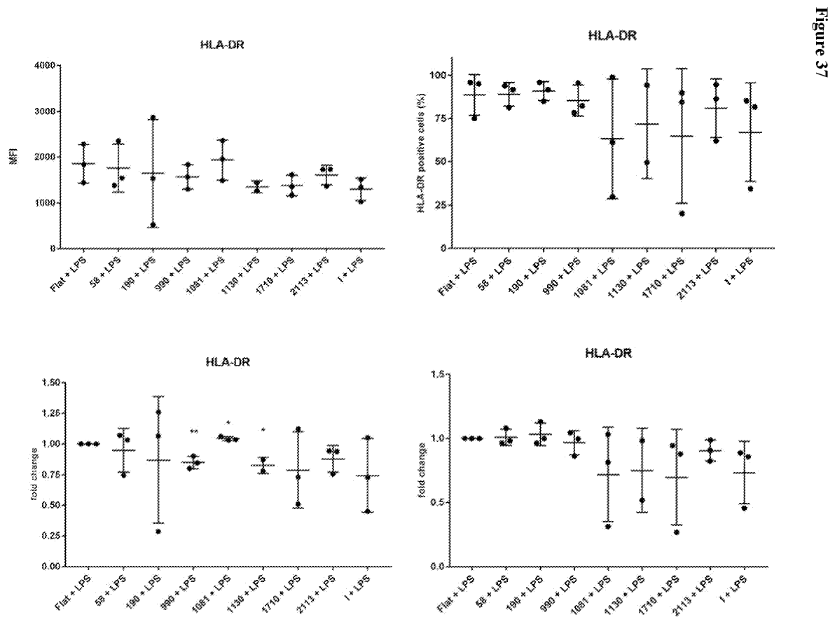

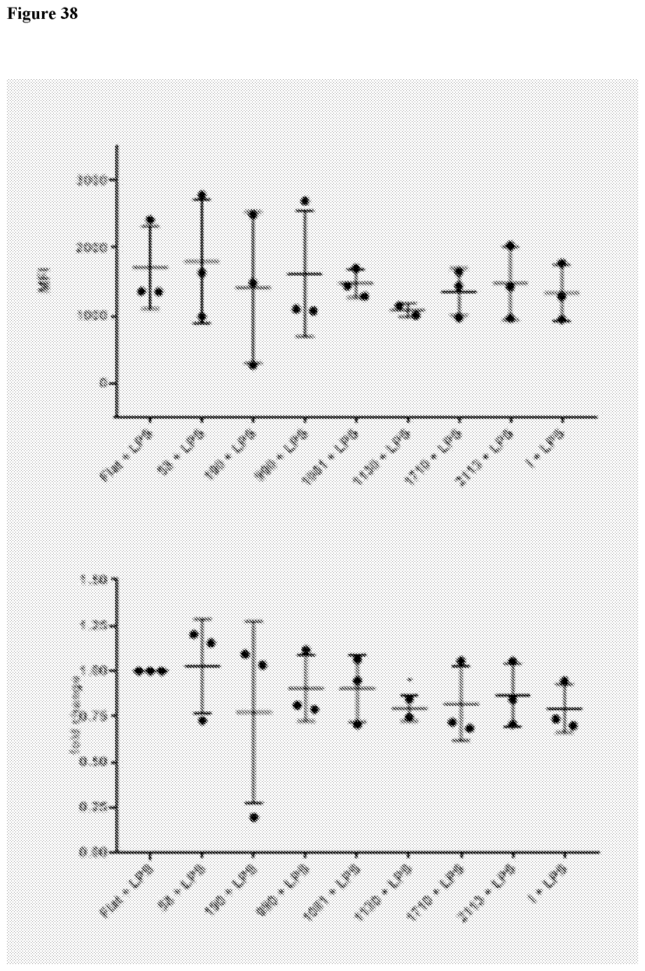

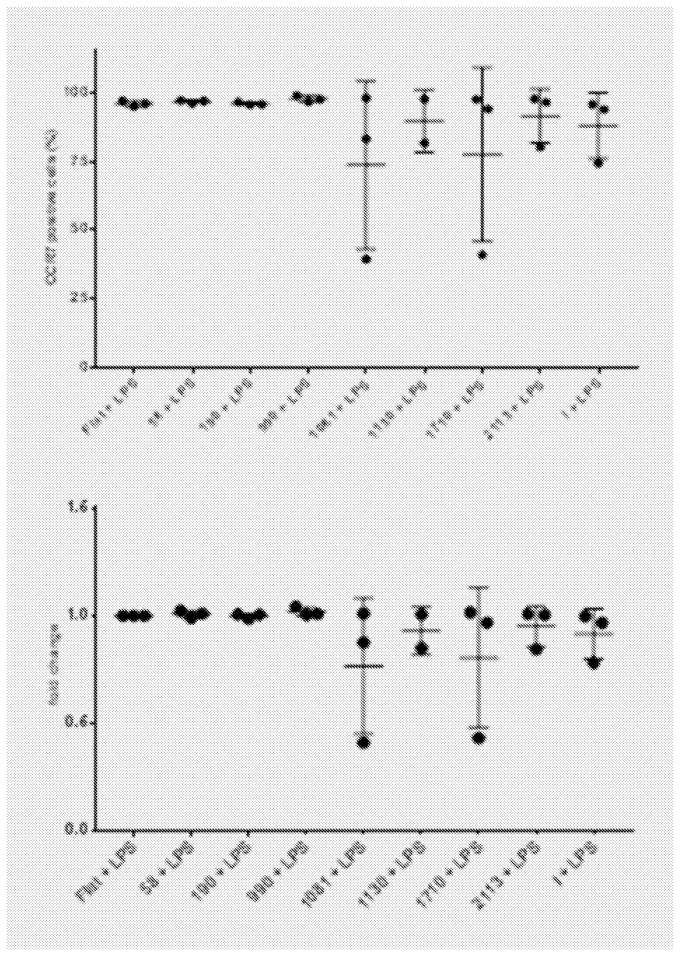

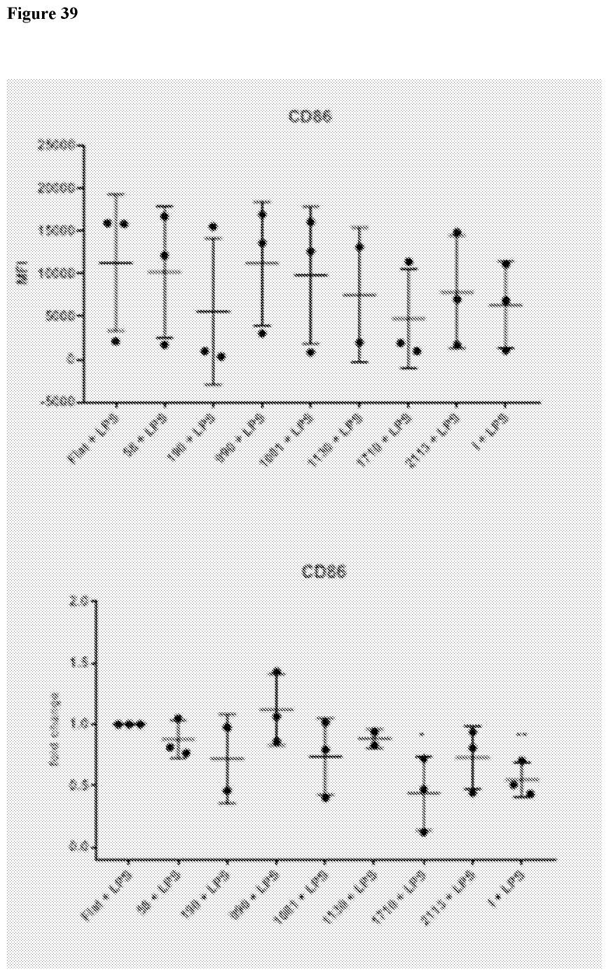

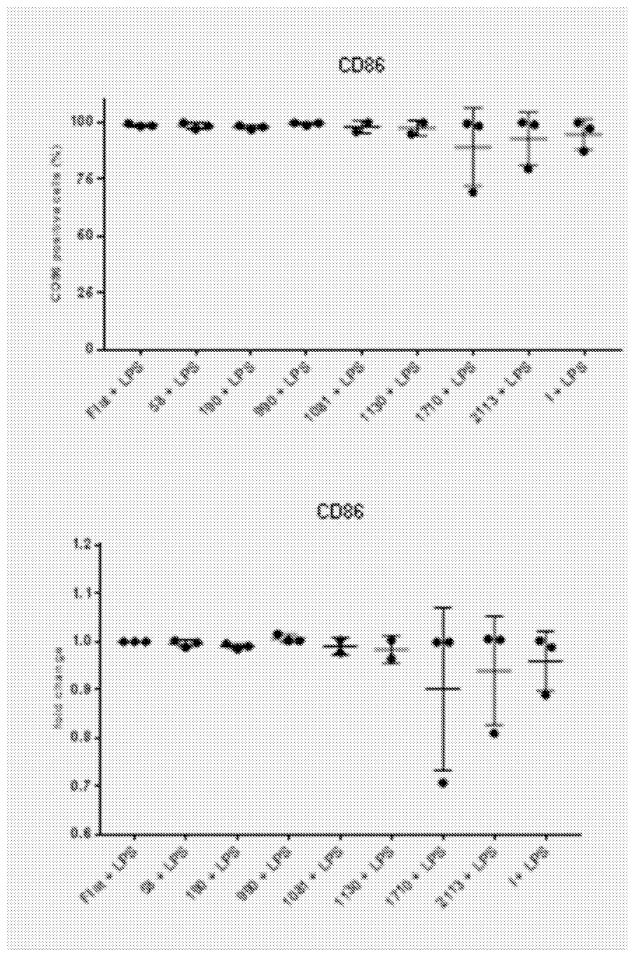

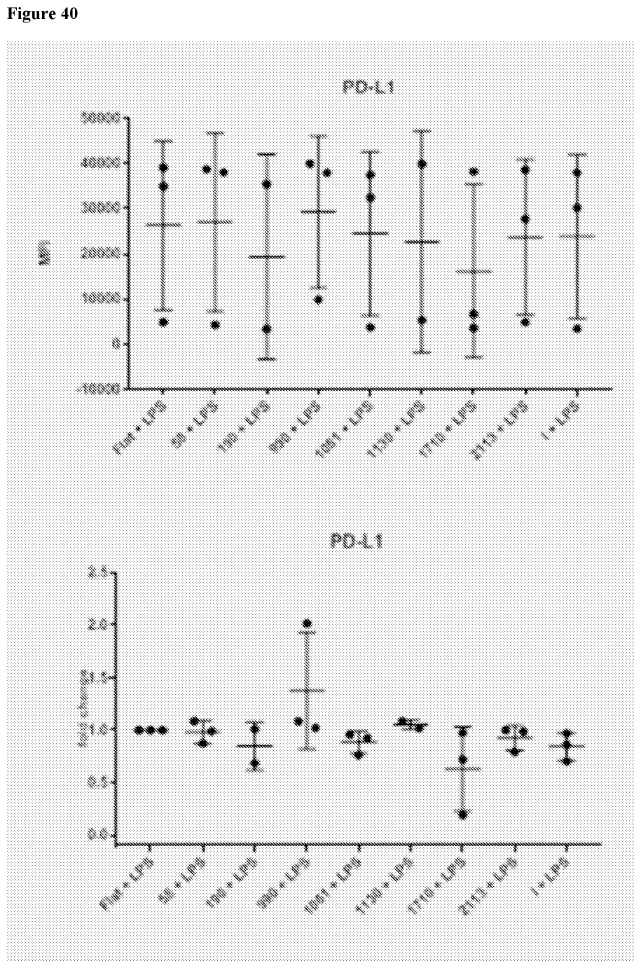

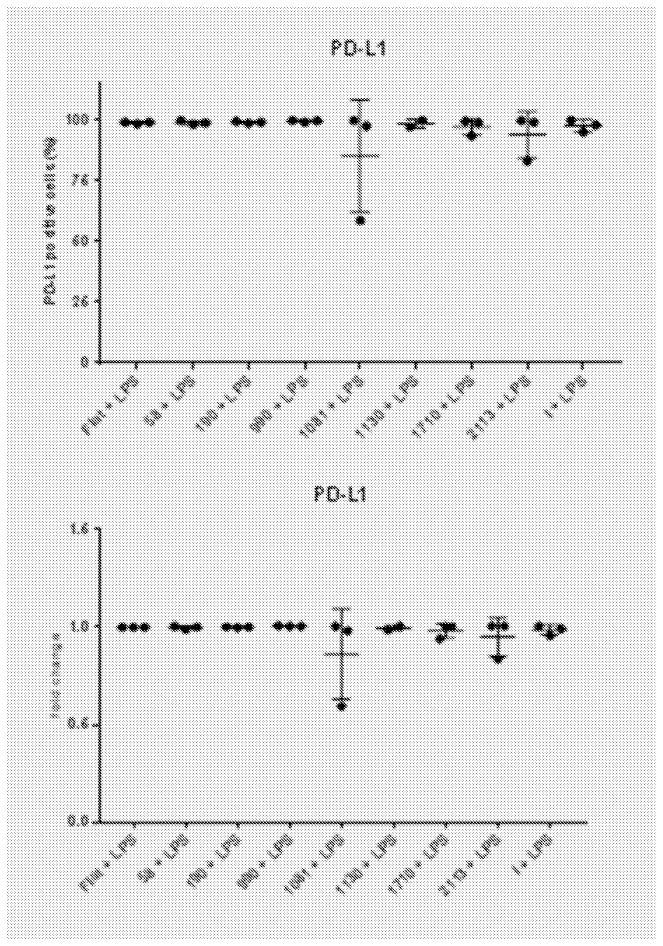

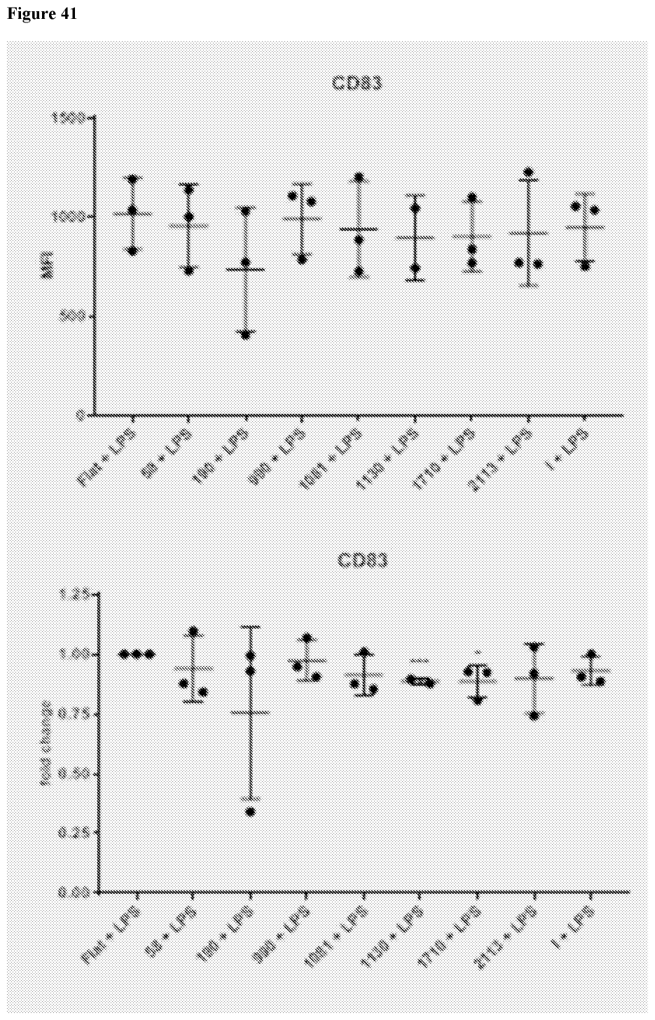

CCR7 expression was slightly downregulated on topography 1130 (FIG. 38)—by circa 20%. CD86 expression was slightly downregulated on topography 1710 and roman number I (FIG. 39)—by circa 50%. PD-L1 expression showed no modulations following LPS stimulation on topographies (FIG. 40). Topographies 1130 and 1710 were both observed to lower expression of CD83 after LPS stimulation on those topographies, compared to flat surface (FIG. 41).

Assessing Cytokine Secretion on Topographies

Following the observations of DC phenotype and movement behaviours, the levels of pro-inflammatory (IL-12p70) and anti-inflammatory (IL-10) cytokines secreted into the culture medium were quantified.

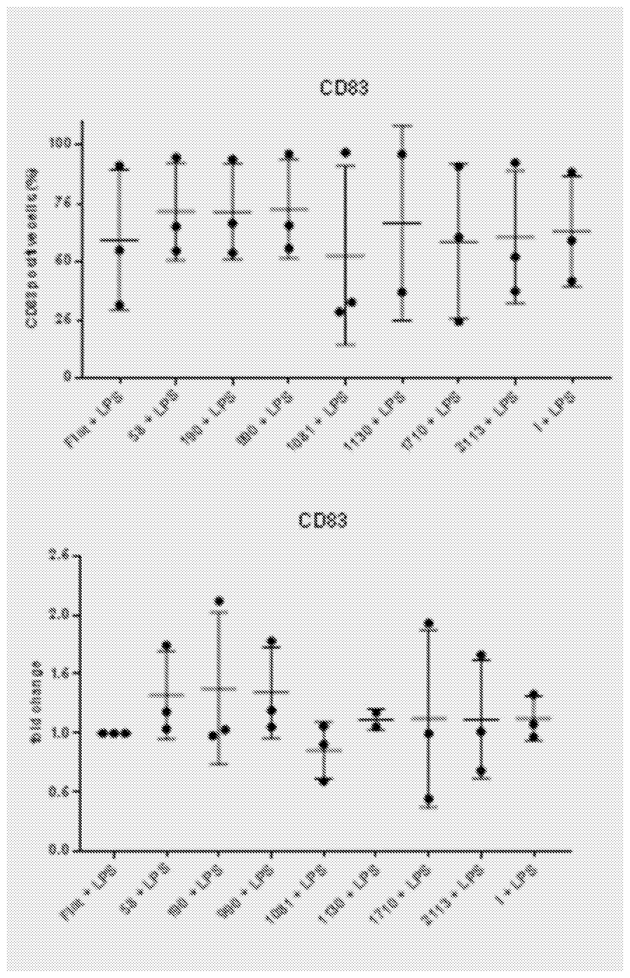

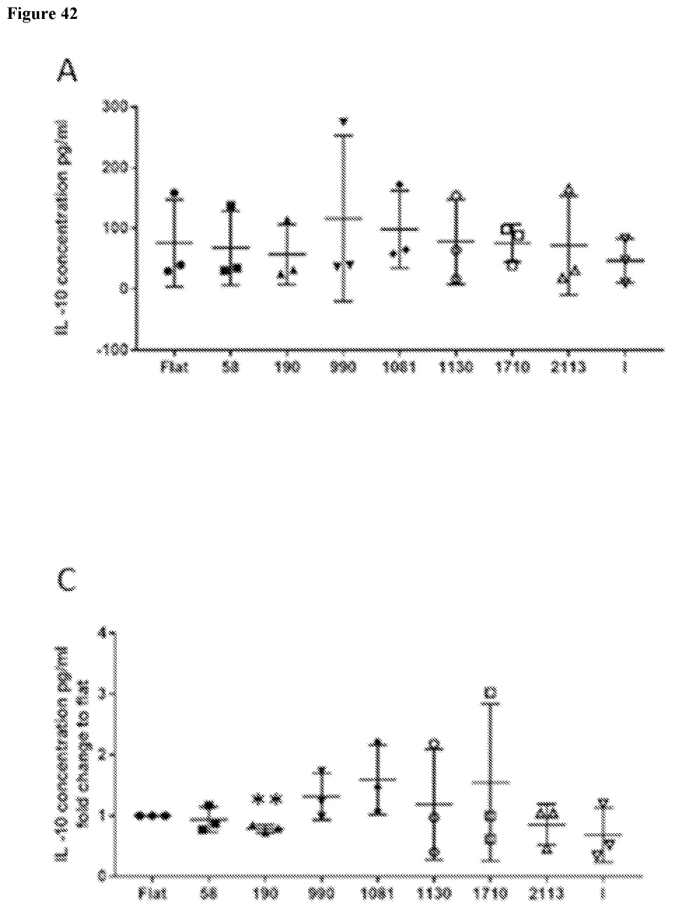

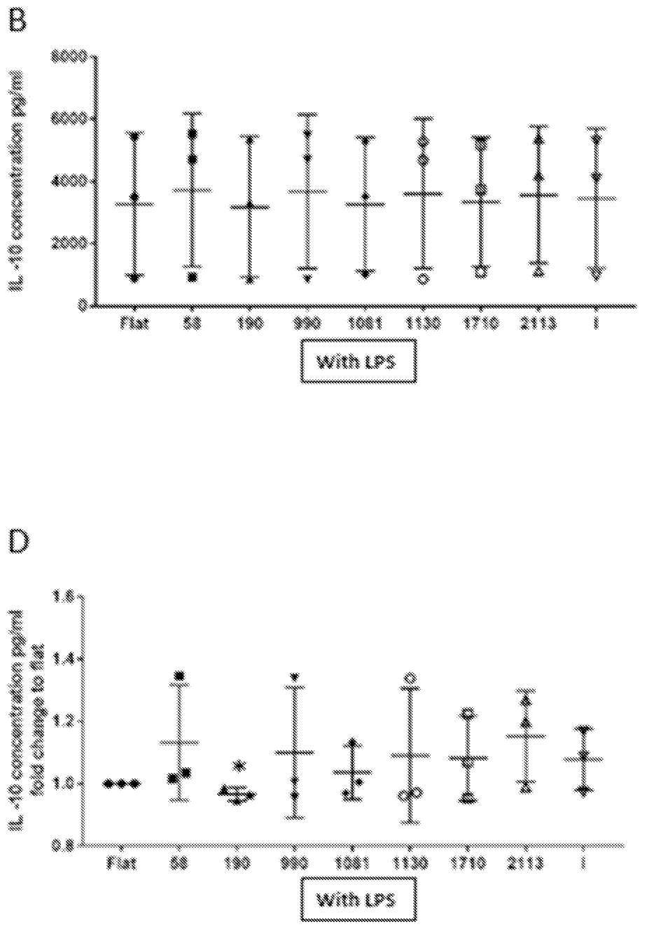

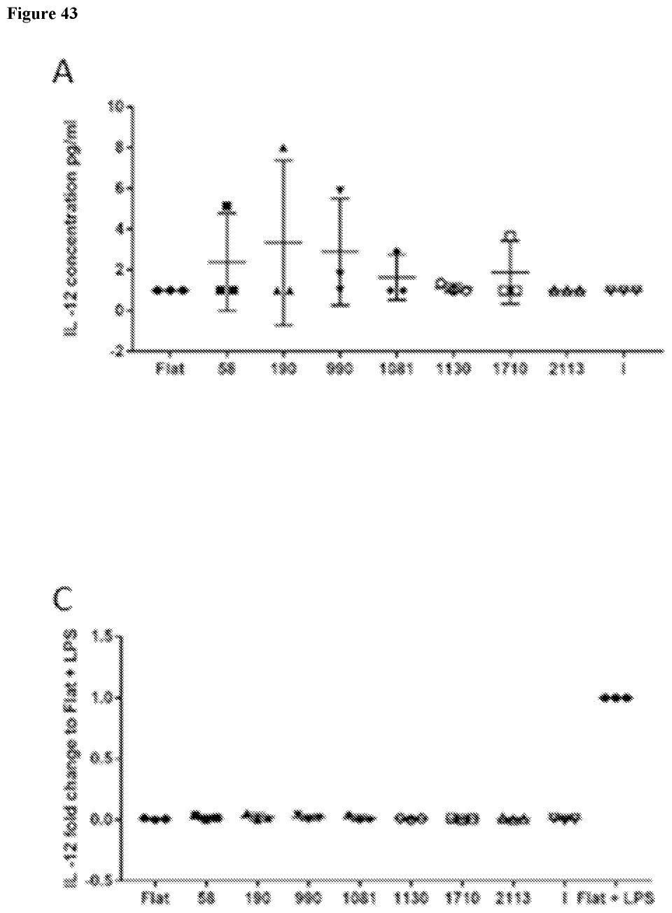

Due to biological variations observed across donors tested, cytokine concentrations were normalised to flat PS control to allow for a more accurate comparison of culture conditions. Following normalisation, no statistically significant differences were observed in the levels of IL-12p70 and IL-10 produced by unstimulated DC on topographic features compared to the flat polystyrene (FIG. 42-43). Only topography 190 very slightly decreased the fold change of IL-10 concentration.

For the stimulated conditions, topography 190 again decreased slightly the fold change for IL-10 concentration between topography and flat polystyrene control (FIG. 42). Topography 1710+LPS induced slight elevated levels of IL-12p70 secretion (FIG. 43).

Effect of Topography Culture on Dendritic Cell Ability to Interact and Initiate T Cell Response

In order to assess the possible functional modulation of dendritic cells by topographic features, the ability of topo-DCs to interact with Pan T cells in a co-culture system was assessed.

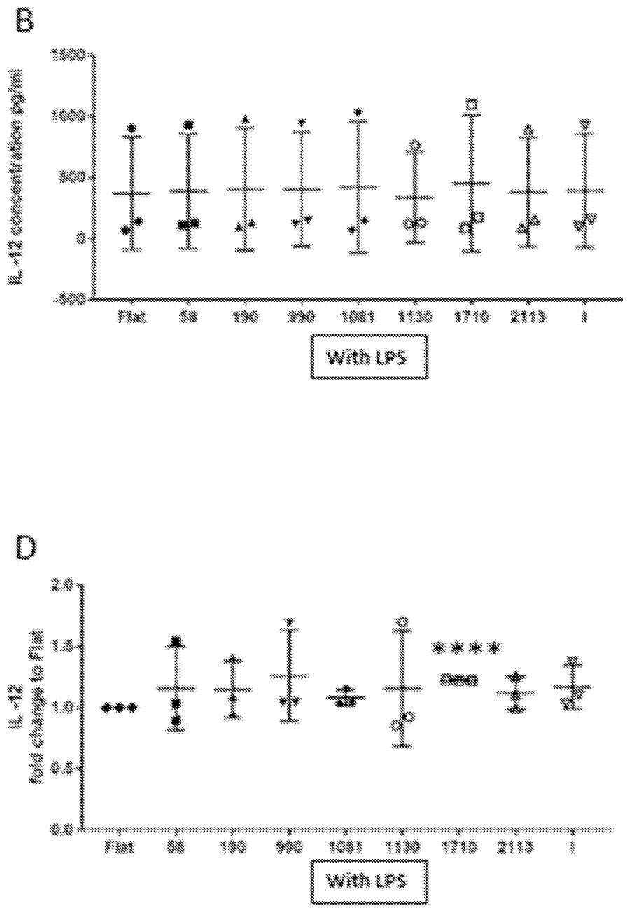

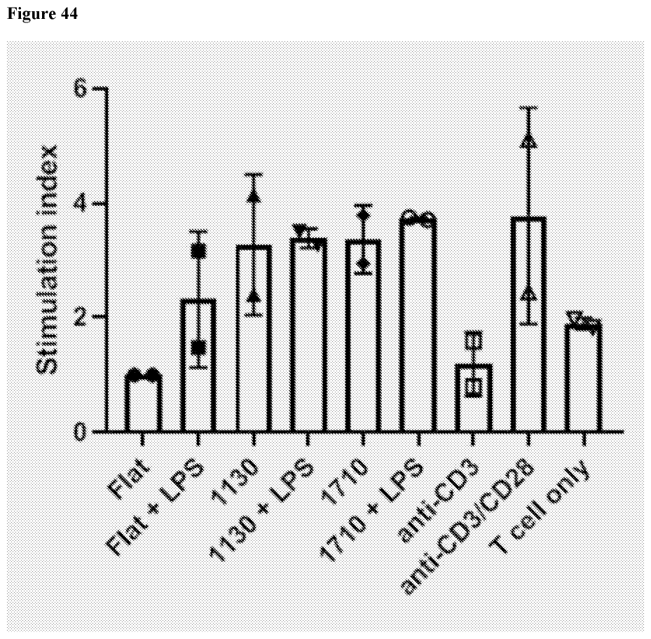

After 8 days of co-culture T cells showed to have proliferated more with topography 1130 and topography 1710-modulated DCs, when compared to flat polystyrene (FIG. 44). The LPS stimulated DCs on the same topographies were on the same proliferation levels as the non-stimulated DCs.

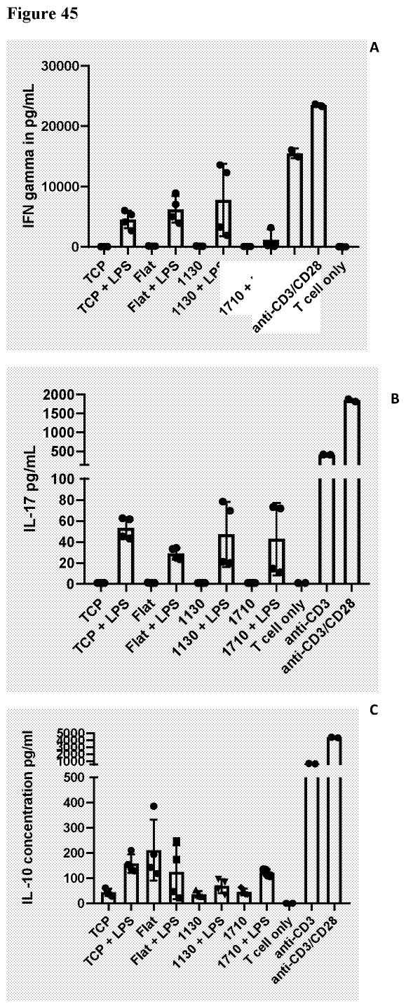

Secretion of IFNgamma was decreased with topography 1701+LPS modulated DCs (FIG. 45a). No modulations in the secretion of IL-17 were observed for topography-modulated DCs-Pan T cell co-cultures. Secretion of IL-10 in the co-cultures of topography-modulated DCs with Pan T cells is inconsistent, and needs further proof/assessment.

### SUMMARY

There is a direct correlation between the spacing in between features and slowing down of DC movement—cells that have tighter spaces to navigate through, will move less and at a much slower speed. Phenotypic investigations show, that topographies decreased the expression of HLA-DR, while other surface markers were not modulated in non-activated DCs. A limited number of topographies inhibited the upregulation of CCR7, CD86 and CD83 slightly; with HLA-DR most potently being modulated when DCs were stimulated with LPS on top the topographies. DC cytokine production did not seem to be modulated by topography culture. Interestingly, topography-modulated DCs were observed to increase the proliferation of Pan T cells in an 8-day co-culture, but did not increase the secretion of IFN gamma and IL-17. Overall, topographies seem to have distinct implications on the antigen-presenting process of DCs. The implication between decreased antigen-presentation of topography-modulated DCs and increased Pan T cell proliferation could have a wide variety of applications.

1. A microtopography system for modulating one or more cellular processes on a surface, said microtopography system comprising:
a repeated microtopographic pattern, said microtopographic pattern comprising:
an array of repeated micropillars applied to a surface of a product, said micropillars being formed of surface structures between 1-100 μm in height, and 1-50 μm in width,
wherein said microtopographic pattern acts to modulate one or more cellular processes on the surface.

2. The system of claim 1, wherein the micropillars are:
about 1-100 μm in height (vertical), such as about between 5-45 μm, 10-40 μm, 15-35 μm, 20-30 μm, 25 μm, or 50-100 μm; and
about 1-100 μm in width (diameter), such as 2-45 μm, 3-40 μm, 4-35 μm, 5-30 μm, 10-25 μm, 15-20 μm, or 50-100 μm.

3. The system of claim 1, wherein the microtopography of the micropillars above the underlying surface have a mean area below 50 μm²; and/or the micropillars have an eccentricity of <1, and preferably less than 0.5, preferably between 0.01-0.49, more preferable between 0.1-0.4, and most preferably between 0.2-0.3.

4. The system of claim 1, wherein the one or more cellular processes comprises of consists of cell attachment and/or immune activity and/or immune activity of cells.

5. The system of claim 4, wherein the cells comprise one or more of innate immune cells, adaptive immune cells or non-immune cells.

6. A product comprising the system of claim 1, wherein said surface comprises a surface of the product and wherein said microtopography modulates cell attachment to the surface of said product and/or immune activity of the attached cells.

7. A product according to claim 6, wherein the system is for preventing or reducing biofilm formation and/or preventing or treating an infection and/or preventing rust formation.

8. A product according to claim 6, wherein the product comprises a wound dressing, a cell culture dish, a food container, packaging or the like, an encasing, a biological catalytic surface for an industrial surface, a food processing product, a water container or treatment product, a beverage container or surface for use in the beverage industry and/or a surface used in displays or windows.

9. A product according to claim 6, wherein the surface is for use in treating or preventing an immune disease/disorder by modulating the attachment and/or immune activity of APCs in a subject.

10. The product of claim 9, wherein the APC is a macrophage or a dendritic cell.

11. The product according to claim 9, wherein the immune disease/disorder is selected from the following: transplant rejection, Graft Versus Host Disease (GVHD), psoriasis, eczema, rheumatoid arthritis, a cancer, immunosuppression, systemic lupus erythematosus, inflammatory bowel disease, Crohn's disease, multiple sclerosis, Type I diabetes, Guillain-Barre syndrome, fibrosis, chronic non-healing wounds or medical device rejection.

12. A product comprising a microtopographical system according to claim 1, for use in preventing or treating diseases selected from the following: transplant rejection, Graft Versus Host Disease (GVHD), psoriasis, eczema, rheumatoid arthritis, a cancer, immunosuppression, systemic lupus erythematosus, inflammatory bowel disease, Crohn's disease, multiple sclerosis, Type I diabetes, Guillain-Barre syndrome, fibrosis, chronic non-healing wounds or medical device rejection.

13. The product for use according to claim 12, wherein the microtopography modulates cell attachment to the surface of said product and/or immune activity of the attached cells

14. The product for use according to claim 12, wherein the disease to be prevented or treated is caused by one or more of a bacteria, a virus, a fungi, a protozoan.

15. The product use according to claim 14, wherein the caused by one or more of a bacteria, a virus, a fungi, a protozoan are caused by one or more of Pseudomonas spp., Staphylococcus spp., Bacillus spp., Lactobacillus sp., Proteus spp., Enterobacter spp., Escherichia coli, Klebsiella spp., Salmonella spp., Listeria spp., Yersinia spp., Legionella spp, Clostridium spp., Acinetobacter spp., Pseudomonas aeruginosa, Staphylococcus aureus, Proteus mirabilis, or Acinetobacter baumannii.

16. The product for use according to claim 12, wherein the product is one of: an implantable medical device, prosthetic, surgical tool, dental tool; dental device; catheter, dental screw, knee joint replacement, hip joint replacement, heart valve replacement, a stent, pacemaker, glucose sensor, contraceptive implant, breast implant, Implantable Cardioverter Defibrillators, spinal screws/rods/artificial discs, contact lenses, shunts stents or wound care products.

17. A method of determining surface topographical descriptors for modulating one or more biological processes on a surface, said method comprising:
providing a surface comprising a plurality of topographical microtopography features, each microtopography feature comprising microtopographical elements, each microtopographical element comprising primitives that repeat to form a microtopographical surface, said microtopographical elements comprising micro-pillars approximately 10 μm high, and said primitives approximately 3 μm wide;
exposing said surface to a biological entity, including one of a cell, bacteria, fungi or the like;
analysing said surface to identify microtopographical elements that provide modulation of the biological processes in the manner desired
identifying one or more descriptors that correspond to the microtopographical elements, wherein each descriptor comprises one of
correlating the descriptors to biological processes; and
providing a surface having descriptors that modulate the biological process of the biological entity.
